SEQUENCE LISTING (33 sequences)

SEQ ID NO: 1 - DNA, 1317 bp, Chikungunya virus
tacgaacacgtaacagtgatcccgaacacggtgggagtaccgtataagactctagtcaacagaccgggctacagccccatggtattggagatggaactactgtcagtcactttggagccaacactatcgcttgattacatcacgtgcgagtacaaaaccgtcatcccgtctccgtacgtgaaatgctgcggtacagcagagtgcaaggacaaaaacctacctgactacagctgtaaggtcttcaccggcgtctacccatttatgtggggcggcgcctactgcttctgcgacgctgaaaatacgcaattgagcgaagcacatgtggagaagtccgaatcatgcaaaacagaatttgcatcagcatacagggctcataccgcatccgcatcagctaagctccgcgtcctttaccaaggaaataacatcactgtaactgcctatgcaaacggcgaccatgccgtcacagttaaggacgccaaattcattgtggggccaatgtcttcagcctggacacctttcgacaacaaaattgtggtgtacaaaggtgacgtctataacatggactacccgccctttggcgcaggaagaccaggacaatttggcgatatccaaagtcgcacacctgagagtaaagacgtctatgctaatacacaactggtactgcagagaccggctgcgggtacggtacacgtgccatactctcaggcaccatctggctttaagtattggctaaaagaacgaggggcgtcgctgcagcacacagcaccatttggctgccaaatagcaacaaacccggtaagagcggtgaactgcgccgtagggaacatgcccatctccatcgacataccggaagcggccttcactagggtcgtcgacgcgccctctttaacggacatgtcgtgcgaggtaccagcctgcacccattcctcagactttgggggcgtcgccattattaaatatgcagccagcaagaaaggcaagtgtgcggtgcattcgatgactaacgccgtcactattcgggaagctgagatagaagttgaagggaattctcagctgcaaatctctttctcgacggccttagccagcgccgaattccgcgtacaagtctgttctacacaagtacactgtgcagccgagtgccaccccccgaaggaccacatagtcaactacccggcgtcacataccaccctcggggtccaggacatttccgctacggcgatgtcatgggtgcagaagatcacgggaggtgtgggactggttgtcgctgttgcagcactgattctaatcgtggtgctatgcgtgtcgttcagcaggcac

SEQ ID NO: 2 - DNA, 2697 bp, Dengue Virus serotype 1
ggcacgggagcccaaggggaaacactgggagagaaatggaaaagacagctgaaccaactgagcaagtcagaattcaacacctacaaaaggagtgggattatggaggtggacagatccgaagccaaagagggactgaaaagaggagaaacaaccaaacatgcagtgtcgagaggaaccgccaaactgaggtggtttgtggagaggaaccttgtgaaaccagaagggaaagtcatagacctcggttgtggaagaggtggctggtcatattattgcgctgggctgaagaaagtcacagaagtgaagggatacacaaaaggaggacctggacatgaggaaccaatcccaatggcgacctatggatggaacctagtaaagctacactccgggaaagatgtattctttataccacctgagaaatgtgacacccttttgtgtgatattggtgagtcctctccgaacccaactatagaagaaggaagaacgttacgtgttctaaagatggtggaaccatggctcagaggaaaccaattttgcataaaaattctaaatccctacatgccaagtgtggtagaaactctggagcaaatgcaaagaaaacatggaggaatgctagtgcgaaatccactctcaagaaattccactcatgaaatgtactgggtttcatgtggaacaggaaacattgtgtcagcagtaaacatgacatccagaatgttgctaaatcgattcacaatggctcacaggaagccaacatatgaaagagacgtggacttaggcgctggaacaagacatgtggcagtggaaccagaggtagccaacctagatatcattggccagaggatagagaacataaaaaatgaacacaagtcaacatggcattatgatgaggacaatccatacaaaacatgggcctatcatggatcatatgaggtcaagccatcaggatcagcctcatccatggtcaatggtgtggtgagactgctcaccaaaccatgggatgtcatccccatggtcacacaaatagccatgactgacaccacaccctttggacaacagagggtgtttaaagagaaagttgacacgcgcacaccaaaagcaaaacgaggcacagcacaaatcatggaggtgacagccaagtggttatggggttttctttctagaaacaaaaaacccagaatctgcacaagagaggagttcacaagaaaagttaggtcaaacgcagccattggagcagtgttcgttgatgaaaatcaatggaactcagcaaaagaagcagtggaagatgaacggttctgggaccttgtgcacagagagagggagcttcataaacagggaaaatgtgccacgtgtgtctacaacatgatggggaagagagagaaaaaactaggagagtttggaaaggcaaaaggaagtcgtgcaatatggtacatgtggttgggagcacgctttctagagttcgaagcccttggtttcatgaatgaagatcactggttcagcagagagaattcactcagtggagtggaaggagaaggactccacaaacttggatacatactcagagacatatcaaagattccagggggaaatatgtatgcagatgacacagccggatgggacacaagaataacagaggatgatcttcagaatgaggccaaaatcactgacatcatggaacctgaacatgccctactggctacgtcaatctttaagctaacctaccaaaataaggtggtaagggtgcagagaccagcaaaaaatggaaccgtgatggatgtcatatccagacgtgaccagagaggaagtggacaggtcggaacttatggcttaaacactttcaccaacatggaggcccaactaataagacaaatggagtctgagggaatcttttcacccagcgaattggaaaccccaaatttagccgagagagttctcgactggttggaaaaacatggcgtcgaaaggctgaaaagaatggcaatcagcggagatgactgcgtggtgaaaccaattgatgacaggttcgcaacagccttaacagctctgaatgacatgggaaaagtaagaaaagacataccgcaatgggaaccttcaaaaggatggaatgattggcaacaagtgcctttctgttcacaccatttccaccagctgattatgaaggatgggagggaaatagtggtgccatgccgcaaccaagatgaacttgtgggtagggctagagtatcacaaggcgccggatggagcctgagagaaactgcatgcctaggcaagtcatatgcacaaatgtggcagctgatgtacttccacaggagagacctgagactagcggctaatgctatctgttcagccgttccagttgattgggtcccaaccagccgcaccacctggtcgatccatgcccaccaccaatggatgacaacagaagacatgttgtcagtgtggaatagggtttggatagaggaaaacccatggatggaggacaaaactcatgtatccagttgggaagatgttccatacctagggaaaagggaagatcaatggtgtggatccctgataggcttaacagcaagggccacctgggccaccaacatacaagtggccataaaccaagtgagaaggctcattgggaatgagaattatctagattacatgacatcaatgaagagattcaagaacgagagtgatcccgaaggggcactctgg

SEQ ID NO: 3 - DNA, 2710 bp, Dengue virus serotype 2
ggaactggcaacataggagagacacttggagaaaaatggaaaagccgattaaacgcactgggaaaaagtgaatttcagatctacaagaaaagtggaatccaggaagtggatagaaccttagcaaaagaaggcatcaaaagaggagaaacggaccaccacgctgtgtcgcgaggctcagcaaaactgagatggttcgtcgagagaaatatggtcacaccagaagggaaggtggtggacctcggttgcggcagagggggctggtcatactattgtgggggactaaagaatgtaagagaagtcaaaggcctaacaaaaggaggaccaggacacgaagaacccatccccatgtcaacatatgggtggaatctagtgcgtctgcaaagtggagttgacgttttcttcaccccgccagaaaagtgtgatacattgttgtgtgacataggggagtcgtcaccaaatcccacgatagaagcaggacgaacactcagagtcctcaacttagtggaaaattggttgaacaataacacccaattttgcataaaggttctcaacccatatatgccctcagtcatagaaaaaatggaaacactacaaaggaaatatggaggagccttagtgaggaatccactctcacgaaactccacacatgagatgtactgggtatccaatgctaccgggaacatagtgtcatcagtgaacatgatttcaaggatgttgattaacagattcacaatgaaacacaagaaagccacctacgagccagatgttgacctaggaagtggaacccgcaacattggaattgaaagtgagataccaaatctagacataataggaaagagaatagagaaaataaaacaagagcatgaaacatcatggcactatgaccaagaccacccatacaaaacgtgggcttaccatggcagctatgaaacaaaacaaactggatcagcatcatctatggtgaacggagtggtcagactgctgacaaaaccttgggacgtcgtccctatggtgacacagatggcaatgacagacacgactccatttggacaacagcgcgttttcaaagagaaagtggacacgagaacccaagaaccgaaggaaggcacaaagaaactgatgaaaatcacggcagagtggctttggaaagaactaggaaagaaaaagacacctaggatgtgtaccagagaagaattcacaagaaaggtgagaagcaatgcagccttgggggccatattcactgatgagaacaaatggaaatcggcacgtgaggctgttgaagatagtaggttttgggagctggttgacagggaaagaaatctccatcttgaaggaaagtgtgaaacatgtgtgtacaacatgatgggaaaaagagagaagaaactaggggagttcggcaaggcaaaaggtagcagagccatatggtacatgtggcttggagcacgcttcttagagtttgaagccctaggattcttgaatgaagatcactggttctccagagggaactccctgagtggagtggaaggagaagggctgcacaggctaggctacattttaagagacgtgagcaagaaggaagggggagcaatgtacgccgatgatacagcaggatgggacacaagaatcacactagaagacttaaaaaatgaagaaatggtaacaaaccacatgaaaggagaacacaagaaactagccgaggccatattcaaattaacgtaccaaaacaaggtggtgcgtgtgcaaagaccaacaccaagaggcacagtaatggatatcatatcgagaagagaccaaagaggcagtgggcaagtcggcacctatggccttaatactttcaccaatatggaagcccaattaattagacagatggagggagaaggaatcttcaaaagcattcagcagcattcagcacctgacagtcacagaagaaatcgctgtacagaactggttagcaagagtggggcgtgaaaggctatcaagaatggccatcagtggagatgattgtgttgtaaaacctttagatgacagatttgcaagtgctttaacagctctaaatgacatgggaaaagttaggaaagatatacaacaatgggaaccttcaagaggatggaacgattggacacaagtgcctttctgttcacaccattttcatgagttagtcatgaaagatggtcgcgtgctcgtagtcccatgcagaaaccaagatgaactgattggtagagcccgaatttcccagggagccgggtggtctttgaaggagacggcctgtttggggaagtcttacgcccaaatgtggaccctgatgtacttccacagacgtgacctcagactggcggcaaatgccatttgctcggcagtcccgtcacattgggttccaacaagtcgaacaacctggtccatacacgctaagcatgaatggatgacgacggaagacatgctggcagtctggaacagggtgtggatccaagaaaacccgtggatggaagacaaaactccagtggaatcatgggaagaagtcccatacttggggaaaagagaagaccaatggtgcggctcattgattgggctaacaagcagggctacctgggcaaagaacatccaaacagcaataaatcaagtcagatcccttataggcaatgaggaatacacagactacatgccatccatgaagagattcagaagggaagaggaagaggcaggtgtcctgtgg

SEQ ID NO: 4 - DNA, 2710 bp, Dengue virus serotype 3
ggaacaggctcacaaggtgaaactttaggagaaaaatggaaaaagaaattaaatcaattatcccggaaagagtttgacctttacaagaaatctggaatcactgaagtggatagaacagaagccaaagaagggttgaaaagaggagaaataacacatcatgccgtgtccagaggtagcgcaaaacttcaatggtttgtggagagaaacatggtcattcccgaaggaagagtcatagacttgggctgtggaagaggaggctggtcatattactgtgcaggactgaaaaaagtcacagaagtgcgaggatacacaaaaggcggtccaggacacgaagaaccagtacctatgtccacatatggatggaacatagttaagttaatgagtggaaaggatgtgttttatcttccacctgaaaagtgtgacaccctgttgtgtgacattggagaatcttcaccaagcccaacagtggaagaaagcagaactataagagttttgaagatggttgaaccatggctaaaaaacaaccagttttgcattaaagtattgaacccttacatgccaactgtgattgagcacctagaaagactacaaaggaaacatggaggaatgcttgtgagaaatccactttcacgaaactccacgcacgaaatgtactggatatctaatggcacaggtaacattgtctcttcagtcaacatggtatctagactgctactgaacaggttcacgatgacacacagaagacccaccatagagaaagatgtggatttaggagcaggaactcgacatgttaatgcggaaccagaaacacccaacatggatgtcattggggaaagaataaaaaggatcaaggaggagcataattcaacatggcactatgatgacgaaaacccctacaaaacgtgggcttaccatggatcttatgaagtcaaagccacaggctcagcctcctccatgataaatggagtcgtgaaactcctcactaaaccatgggatgtggtgcccatggtgacacagatggcaatgacagatacaactccatttggccagcagagagtctttaaagagaaagtggacaccaggacacccaggcccatgccaggaacaagaaaggttatggagatcacagcggagtggctctggagaaccctgggaaggaacaaaaaacccaggttatgcacaagggaagagtttacaaaaaaggtcagaactaacgcagccatgggcgccgttttcacagaggagaaccaatgggacagcgcgaaagctgctgttgaggatgaggatttttggaaacttgtggacagagaacgtgaactccacaaattgggcaagtgtggaagctgtgtttacaacatgatgggcaagagagagaagaaacttggagagtttggcaaagcaaaaggcagtagagctatatggtacatgtggttgggagccaggtaccttgagttcgaagcccttggattcttaaatgaagaccactggttctcgcgtgagaactcttacagtggagtagaaggagaaggactgcacaagctaggctatatattaagggacatttccaagatacccggaggagctatgtatgctgatgacacagctggttgggacacaagaataacagaagatgacctgcacaatgaggaaaagatcacacagcaaatggaccctgaacacaggcagttagcgaacgctatatttaagctcacataccaaaacaaagtggtcaaagttcaacgaccgactccaacaggcacggtaatggacatcatatctaggaaagaccaaagaggcagtggacaggtgggaacttatggtctgaatacattcaccaacatggaagcccagttaatcagacaaatggaaggagaaggtgtgctgtcaaaggcagacctcggcagacctcgagaaccctcatctgccagagaagaaaattacacaatggttggaaaccaaaggagtggagaggttaaaaagaatggccattagcggggatgattgcgtagtgaaaccaatcgatgacaggttcgctaatgccctgcttgctctgaacgatatgggaaaggttcggaaagacatacctcaatggcagccatcaaagggatggcatgattggcaacaggttcctttctgctcccaccactttcatgaattgatcatgaaagatggaagaaagttggtggttccctgcagaccccaggacgaactaataggaagagcaagaatctctcaaggagcgggatggagccttagagaaaccgcatgtctggggaaagcctacgctcaaatgtggagtctcatgtattttcacagaagagatctcagactagcatccaacgccatatgttcagcagtaccagtccactgggtccccacaagtagaacgacatggtctattcatgctcaccatcagtggatgaccacagaagacatgcttactgtctggaacagggtgtggatcgaggacaatccatggatggaagacaaaactccagtcacaacctgggaaaatgttccatatctagggaagagagaagaccaatggtgcggatcacttattggtctcacttccagagcaacctgggcccagaacatacccacagcaattcaacaggtgagaagccttataggcaatgaagagtttctggactacatgccttcaatgaagagattcaggaaggaggaggagtcggagggagccatttgg

SEQ ID NO: 5 - DNA, 339 bp, Dengue virus serotype 4
atgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgctgtgcttgattcccaccgtaatggcg

SEQ ID NO: 6 - DNA, 2719 bp, Zika virus
gggggtggaacaggagagaccctgggagagaaatggaaggcccgcttgaaccagatgtcggccctggagttctactcctacaaaaagtcaggcatcaccgaggtgtgcagagaagaggcccgccgcgccctcaaggacggtgtggcaacgggaggccatgctgtgtcccgaggaagtgcaaagctgagatggttggtggagcggggatacctgcagccctatggaaaggtcattgatcttggatgtggcagagggggctggagttactacgccgccaccatccgcaaagttcaagaagtgaaaggatacacaaaaggaggccctggtcatgaagaacccgtgttggtgcaaagctatgggtggaacatagtccgtcttaagagtggggtggacgtctttcatatggcggctgagccgtgtgacacgttgctgtgtgacataggtgagtcatcatctagtcctgaagtggaagaagcacggacgctcagagtcctctccatggtgggggattggcttgaaaaaagaccaggagccttttgtataaaagtgttgtgcccatacaccagcactatgatggaaaccctggagcgactgcagcgtaggtatgggggaggactggtcagagtgccactctcccgcaactctacacatgagatgtactgggtctctggagcgaaaagcaacaccataaaaagtgtgtccaccacgagccagctcctcttggggcgcatggacgggcctaggaggccagtgaaatatgaggaggatgtgaatctcggctctggcacgcgggctgtggtaagctgcgctgaagctcccaacatgaagatcattggtaaccgcattgaaaggatccgcagtgagcacgcggaaacgtggttctttgacgagaaccacccatataggacatgggcttaccatggaagctatgaggcccccacacaagggtcagcgtcctctctaataaacggggttgtcaggctcctgtcaaaaccctgggatgtggtgactggagtcacaggaatagccatgaccgacaccacaccgtatggtcagcaaagagttttcaaggaaaaagtggacactagggtgccagacccccaagaaggcactcgtcaggttatgagcatggtctcttcctggttgtggaaagagctaggcaaacacaaacggccacgagtctgtaccaaagaagagttcatcaacaaggttcgtagcaatgcagcattaggggcaatatttgaagaggaaaaagagtggaagactgcagtggaagctgtgaacgatccaaggttctgggctctagtggacaaggaaagagagcaccacctgagaggagagtgccagagttgtgtgtacaacatgatgggaaaaagagaaaagaaacaaggggaatttggaaaggccaagggcagccgcgccatctggtatatgtggctaggggctagatttctagagttcgaagcccttggattcttgaacgaggatcactggatggggagagagaactcaggaggtggtgttgaagggctgggattacaaagactcggatatgtcctagaagagatgagtcgcataccaggaggaaggatgtatgcagatgacactgctggctgggacacccgcatcagcaggtttgatctggagaatgaagctctaatcaccaaccaaatggagaaagggcacagggccttggcattggccataatcaagtacacataccaaaacaaagtggtaaaggtccttagaccagctgaaaaagggaaaacagttatggacattatttcgagacaagaccaaagggggagcggacaagttgtcacttacgctcttaacacatttaccaacctagtggtgcaactcattcggaatatggaggctgaggaagttctagagatgcctagagatgcaagacttgtggctgctgcggaggtcagagaaagtgaccaactggttgcagagcaacggatgggataggctcaaacgaatggcagtcagtggagatgattgcgttgtgaagccaattgatgataggtttgcacatgccctcaggttcttgaatgatatgggaaaagttaggaaggacacacaagagtggaaaccctcaactggatgggacaactgggaagaagttccgttttgctcccaccacttcaacaagctccatctcaaggacgggaggtccattgtggttccctgccgccaccaagatgaactgattggccgggcccgcgtctctccaggggcgggatggagcatccgggagactgcttgcctagcaaaatcatatgcgcaaatgtggcagctcctttatttccacagaagggacctccgactgatggccaatgccatttgttcatctgtgccagttgactgggttccaactgggagaactacctggtcaatccatggaaagggagaatggatgaccactgaagacatgcttgtggtgtggaacagagtgtggattgaggagaacgaccacatggaagacaagaccccagttacgaaatggacagacattccctatttgggaaaaagggaagacttgtggtgtggatctctcatagggcacagaccgcgcaccacctgggctgagaacattaaaaacacagtcaacatggtgcgcaggatcataggtgatgaagaaaagtacatggactacctatccacccaagttcgctacttgggtgaagaagggtctacacctggagtgctg

SEQ ID NO: 7 - DNA, 25 bp, Artificial sequence
  Synthetic sequence
ccttggattcttgaacgaggatcac

SEQ ID NO: 8 - DNA, 24 bp, Artificial sequence
  Synthetic sequence
taccaggaggaaggatgtatgcag

SEQ ID NO: 9 - DNA, 25 bp, Artificial sequence
  Synthetic sequence
gcttcattctccagatcaaacctgc

SEQ ID NO: 10 - DNA, 21 bp, Artificial sequence
  Synthetic sequence
ggctgaagaaagtcacagaag

SEQ ID NO: 11 - DNA, 21 bp, Artificial sequence
  Synthetic sequence
ggctgaagaaagtcactgaag

SEQ ID NO: 12 - DNA, 26 bp, Artificial sequence
  Synthetic sequence
acctatggatggaacctagtaaagct

SEQ ID NO: 13 - DNA, 23 bp, Artificial sequence
  Synthetic sequence
gaggactcaccaatatcacacaa

SEQ ID NO: 14 - DNA, 24 bp, Artificial sequence
  Synthetic sequence
gctcagcctcctccatgataaatg

SEQ ID NO: 15 - DNA, 24 bp, Artificial sequence
  Synthetic sequence
catggtgacacagatggcaatgac

SEQ ID NO: 16 - DNA, 24 bp, Artificial sequence
  Synthetic sequence
cacggtgacacagatggcaatgac

SEQ ID NO: 17 - DNA, 24 bp, Artificial sequence
  Synthetic sequence
gggtgtcctggtgtccactttctc

SEQ ID NO: 18 - DNA, 21 bp, Artificial sequence
  Synthetic sequence
ggcgcctactgcttctgcgac

SEQ ID NO: 19 - DNA, 24 bp, Artificial sequence
  Synthetic sequence
agcgaagcacatgtggagaagtcc

SEQ ID NO: 20 - DNA, 24 bp, Artificial sequence
  Synthetic sequence
agcgaagcacacgtggagaagtcc

SEQ ID NO: 21 - DNA, 24 bp, Artificial sequence
  Synthetic sequence
ttggtaaaggacgcggagcttagc

SEQ ID NO: 22 - DNA, 24 bp, Artificial sequence
  Synthetic sequence
acacagatggcaatgacagacacg

SEQ ID NO: 23 - DNA, 28 bp, Artificial sequence
  Synthetic sequence
tggaaagaactaggaaagaaaaagacac

SEQ ID NO: 24 - DNA, 22 bp, Artificial sequence
  Synthetic sequence
ccaaggctgcattgcttctcac

SEQ ID NO: 25 - DNA, 23 bp, Artificial sequence
  Synthetic sequence
tggttagaccacctttcaatatg

SEQ ID NO: 26 - DNA, 23 bp, Artificial sequence
  Synthetic sequence
tggctagaccacctttcaatatg

SEQ ID NO: 27 - DNA, 24 bp, Artificial sequence
  Synthetic sequence
cctcaagggttggtgaagagattc

SEQ ID NO: 28 - DNA, 18 bp, Artificial sequence
  Synthetic sequence
tgctagcaccatccgtaa

SEQ ID NO: 29 - DNA, 19 bp, Artificial sequence
  Synthetic sequence
ggcacccagcacaatgaag

SEQ ID NO: 30 - DNA, 28 bp, Artificial sequence
  Synthetic sequence
tcaagatcattgctcctcctgagagcgc

SEQ ID NO: 31 - DNA, 20 bp, Artificial sequence
  Synthetic sequence
gccgatccacacggagtact

SEQ ID NO: 32 - DNA, 25 bp, Artificial sequence
  Synthetic sequence
gcttcattctctagatcaaacctgc

SEQ ID NO: 33 - DNA, 23 bp, Artificial sequence
  Synthetic sequence
accaggaggaaagatgtacgcag

(19) United States
(12) Patent Application Publication
(10) Pub. No.: US 2022/0334113 A1
(43) Pub. Date: Oct. 20, 2022

(54) A Method of Detecting or Differentiating Chikungunya, Dengue, and Zika Viruses

(71) Applicant: Agency for Science, Technology and Research, Singapore (SG)

(72) Inventors: Fong Poh Lisa Ng, Singapore (SG); Jia Lin Jeslin Tan, Singapore (SG)

(21) Appl. No.: 17/764,689

(22) PCT Filed: Oct. 3, 2019
(86) PCT No.: PCT/SG2019/050499

§ 371 Date: Mar. 29, 2022

(51) Int. Cl. G01N 33/569; C12Q 1/70; C12Q 1/6818

(52) U.S. Cl. CPC G01N 33/56983; C12Q 1/701; C12Q 1/6818

(57) ABSTRACT

Disclosed is a method of simultaneously detecting, differentiating, and/or quantifying Chikunguynya virus (CHIKV), Dengue virus serotype-1 (DENV1), Dengue virus serotype-2 (DENV2), Dengue vims serotype-3 (DENV3), Dengue vims serotype-4 (DENV4) and Zika virus (ZIKV) in a sample. In some examples, the method comprises the step of determining the presence of the target regions or fragments thereof selected from the group consisting of Non Structural protein 5 (NS5) of Zika virus, NS5 of DENV1, NS5 of DENV2, NS5 of DENV3, Capsid of DENV4, and E1 glycoprotein of CHIKV. Also disclosed are isolated oligonucleotides for use in methods thereof, methods for detecting and/or differentiating and/or quantifying vims as described herein, and kits for use thereof.

### TECHNICAL FIELD

The present disclosure relates to methods of investigating the presence of viruses in a biological sample. In particular, the methods of the present disclosure detect, differentiate, and/or quantify the presence of one or more viruses including Chikungunya virus, Dengue virus serotype 1, Dengue virus serotype 2, Dengue virus serotype 3, Dengue virus serotype 4, and Zika virus.

### BACKGROUND

Various arboviruses have been the cause of significant epidemics around the world in recent years. Three of the arboviruses that have been prevalent in several countries around the world are Chikungunya virus, Dengue virus, and Zika virus.

Chikungunya Virus (CHIKV), an RNA virus in the genus Alphavirus of the family Togoviridae causes Chikungunya fever, which is characterized by a sudden fever accompanied by skin rashes, joint pain and persistent rheumatic symptoms.

Dengue virus belonging to the family Flaviviridae of the genus Flavivirus has four serotypes, namely Dengue virus serotype 1 (DENV-1), Dengue virus serotype 2 (DENV-2), Dengue virus serotype 3 (DENV-3), and Dengue virus serotype 4 (DENV-4). Dengue fever is a febrile disease with clinical symptoms that may include any one of high fever, severe headache, pain behind the eyes, muscle and joint pains, nausea, vomiting, swollen glands and rash. Infection with one dengue serotype confers lifetime immunity against that serotype. However, whilst the four dengue serotypes are antigenically similar, they are distinct such that infection with one dengue serotype does not confer immunity against all serotypes.

Additionally, subsequent infections by other serotypes can increase the risk of developing severe clinical manifestations.

Zika fever is a viral disease caused by an RNA virus belonging to the family of Flaviviridae of the family Flavivirus. Zika is characterized by fever, exanthema, and non-purulent conjunctivitis.

All three viruses, Chikungunya virus, Dengue virus, and Zika virus are most commonly transmitted by their common vector, the Aedes mosquitoes. Large range of Aedes species, including Aedes aegypti and Aedes albopictus, are very well adapted to living in both urban and rural areas in tropical or subtropical regions such as Asia and Africa. However, outbreaks occurring in temperate climates have also been reported, mainly thought to be due to Aedes albopictus wider geographical distribution.

With many similar and overlapping disease manifestations, symptoms and choice of vector transmission, it is currently a challenge for clinicians to distinguish the causative arbovirus in a patient. Therefore, there is a need to provide a method capable of detecting, distinguishing and/or differentiating Chikungunya virus, the four serotypes of Dengue virus, and Zika virus.

### SUMMARY

In one aspect, there is provided a method of simultaneously detecting, differentiating, and/or quantifying Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, wherein the method comprising: determining the presence of the target regions or fragments thereof selected from the group consisting of Non Structural protein 5 (NS5) of Zika virus, NS5 of DENV1, NS5 of DENV2, NS5 of DENV3, Capsid of DENV4, and E1 glycoprotein of CHIKV.

In some examples, the target regions or fragments thereof are encoded by the sequence SEQ ID NO: 1 (CHIKV E1 consensus sequence); SEQ ID NO: 2 (DENV1 NS5 48+22 seq consensus sequence); SEQ ID NO: 3 (DENV2 NS5 consensus sequence); SEQ ID NO: 4 (DENV3 NS5 consensus sequence); SEQ ID NO: 5 (DENV4 Capsid consensus sequence); and SEQ ID NO: 6 (ZIKV NS5 check_56seq consensus sequence).

In some examples, the detecting comprises performing reverse transcription polymerase chain reaction (RT-PCR).

In some examples, the primers and probe are selected from the group consisting of:

a ZIKV forward primer comprising a
sequence at least 90% identical to
CCTTGGATTCTTGAACGAGGATCAC
(NS5_ZIKV-F/SEQ ID NO: 7);
a ZIKV reverse primer comprising a
sequence at least 90% identical to
GCTTCATTCTCCAGATCAAACCTGC
(NS5_ZIKV-R/SEQ ID NO: 9)
or
GCTTCATTCTCTAGATCAAACCTGC
(ZIKV-R1_T/SEQ ID NO: 32);
a ZIKV probe comprising a sequence
at least 90% identical to
TACCAGGAGGAAGGATGTATGCAG
(5′-FAM NS5_ZIKV-P/ZEN/3′ IBFQ/SEQ ID NO: 8)
or
ACCAGGAGGAAAGATGTACGCAG
(ZIKV_P1_AF/SEQ ID NO: 33);
a DENV1 first forward primer comprising a
sequence at least 90% identical to
GGCTGAAGAAAGTCACAGAAG
(NS5_D1-F_A/SEQ ID NO: 10);
a DENV1 second forward primer comprising
a sequence at least 90% identical to
GGCTGAAGAAAGTCACTGAAG
(NS5_D1-F_T/SEQ ID NO: 11);
a DENV1 reverse primer comprising a
sequence at least 90% identical to
GAGGACTCACCAATATCACACAA
(NS5_D1-R/SEQ ID NO: 13);
a DENV1 probe comprising a sequence
at least 90% identical to
ACCTATGGATGGAACCTAGTAAAGCT
(5′-HEX NS5_D1/ZEN/3′ IBFQ/SEQ ID NO: 12);
a DENV3 forward primer comprising a
sequence at least 90% identical to
GCTCAGCCTCCTCCATGATAAATG
(NS5_D3-F/SEQ ID NO: 14);
a DENV3 reverse primer comprising a
sequence at least 90% identical to
GGGTGTCCTGGTGTCCACTTTCTC
(NS5_D3-R/SEQ ID NO: 17);
a DENV3 first probe comprising a
sequence at least 90% identical to
CATGGTGACACAGATGGCAATGAC
(5′-Texas Red NS5_D3-P_T/3′ IBRQ/SEQ ID NO: 15);
a DENV3 second probe comprising a
sequence at least 90% identical to
CACGGTGACACAGATGGCAATGAC
(5′-Texas Red NS5_D3-P_C/3′ IBRQ/SEQ ID NO: 16);
a CHIKV forward primer comprising a
sequence at least 90% identical to
GGCGCCTACTGCTTCTGCGAC
(E1_CHIKV-F1/SEQ ID NO: 18);
a CHIKV reverse primer comprising a
sequence at least 90% identical to
TTGGTAAAGGACGCGGAGCTTAGC
(E1_CHIKV-R1/SEQ ID NO: 21);
a CHIKV first probe
comprising a sequence at least 90% identical to
AGCGAAGCACATGTGGAGAAGTCC
(5′-FAM E1_CHIKV-P1_T/ZEN/3′ IBFQ/SEQ ID NO: 19);
a CHIKV second probe
comprising a sequence at least 90% identical to
AGCGAAGCACACGTGGAGAAGTCC
(5′-FAM E1_CHIKV-P1_C/ZEN/3′ IBFQ/SEQ ID NO: 20);
a DENV2 forward primer
comprising a sequence at least 90% identical to
ACACAGATGGCAATGACAGACACG
(NS5_D2-F2/SEQ ID NO: 22);
a DENV2 reverse primer
comprising a sequence at least 90% identical to
CCAAGGCTGCATTGCTTCTCAC
(NS5_D2-R2/SEQ ID NO: 24);
a DENV2 probe
comprising a sequence at least 90% identical to
TGGAAAGAACTAGGAAAGAAAAAGACAC
(5′-HEX NS5_D2-P2/ZEN/3′ IBFQ/SEQ ID NO: 23);
a DENV4 first forward primer
comprising a sequence at least 90% identical to
TGGTTAGACCACCTTTCAATATG
(C_D4-F1.2_T/SEQ ID NO: 25);
a DENV4 second forward primer
comprising a sequence at least 90% identical to
TGGCTAGACCACCTTTCAATATG
(C_D4-F1.2_C/SEQ ID NO: 26);
a DENV4 reverse primer
comprising a sequence at least 90% identical to
TGCTAGCACCATCCGTAA
(C_D4-R1.2/SEQ ID NO: 28);
a DENV4 probe
comprising a sequence at least 90% identical to
CCTCAAGGGTTGGTGAAGAGATTC
(5′-Texas Red C_D4-P1/3′ IBRQ/SEQ ID NO: 27);
and a combination thereof.

In some examples, the primers and probes are conjugated with a detectable label. In some examples, the detectable label may include, but is not limited to a fluorophore, a quencher, a combination thereof, and the like.

In some examples, the sample is selected from the group consisting of whole blood, serum, plasma, cerebrospinal fluid, urine, and amniotic fluid.

In some examples, the sample is whole blood.

In some examples, the sample is whole blood treated with EDTA.

In one aspect, there is provided an isolated oligonucleotide for a simultaneous detection and/or differentiation and/or quantification of virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, wherein the oligonucleotide detects a nucleic acid sequence that is at least 80% identical to the sequences selected from the group consisting of: a nucleic acid molecule that encodes a nucleotide sequence of Non Structural protein 5 (NS5) of Zika virus, a nucleic acid molecule that encodes a nucleotide sequence of NS5 of DENV1, a nucleic acid molecule that encodes a nucleotide sequence of NS5 of DENV2, a nucleic acid molecule that encodes a nucleotide sequence of NS5 of DENV3, a nucleic acid molecule that encodes a nucleotide sequence of Capsid of DENV4, and a nucleic acid molecule that encodes a nucleotide sequence of E1 glycoprotein.

In some examples, the nucleotide sequence of E1 glycoprotein of CHIKV comprises SEQ ID NO: 1 or its fragment thereof, the nucleotide sequence of NS5 of DENV1 comprises SEQ ID NO: 2 or its fragment thereof, the nucleotide sequence of NS5 of DENV2 comprises SEQ ID NO: 3 or its fragment thereof, the nucleotide sequence of NS5 of DENV3 comprises SEQ ID NO: 4 or its fragment thereof, the nucleotide sequence of Capsid of DENV4 comprises SEQ ID NO: 5 or its fragment thereof, and the nucleotide sequence of Non Structural protein 5 (NS5) of Zika virus comprises SEQ ID NO: 6 or its fragment thereof.

In some examples, the oligonucleotide comprises:

a ZIKV forward primer comprising a sequence at least 90% identical to CCTTGGATTCTTGAACGAGGATCAC (SEQ ID NO: 7);

a ZIKV reverse primer comprising a sequence at least 90% identical to GCTTCATTCTCCAGATCAAACCTGC (SEQ ID NO: 9) or GCTTCATTCTCTAGATCAAACCTGC (SEQ ID NO: 32);

a ZIKV probe comprising a sequence at least 90% identical to TACCAGGAGGAAGGATGTATGCAG (SEQ ID NO: 8) or ACCAGGAGGAAAGATGTACGCAG (SEQ ID NO: 33);

a DENV1 first forward primer comprising a sequence at least 90% identical to GGCTGAAGAAAGTCACAGAAG (SEQ ID NO: 10);

a DENV1 second forward primer comprising a sequence at least 90% identical to GGCTGAAGAAAGTCACTGAAG (SEQ ID NO: 11);

a DENV1 reverse primer comprising a sequence at least 90% identical to GAGGACTCACCAATATCACACAA (SEQ ID NO: 13);

a DENV1 probe comprising a sequence at least 90% identical to ACCTATGGATGGAACCTAGTAAAGCT (SEQ ID NO: 12);

a DENV3 forward primer comprising a sequence at least 90% identical to GCTCAGCCTCCTCCATGATAAATG (SEQ ID NO: 14);

a DENV3 reverse primer comprising a sequence at least 90% identical to GGGTGTCCTGGTGTCCACTTTCTC (SEQ ID NO: 17);

a DENV3 first probe comprising a sequence at least 90% identical to CATGGTGACACAGATGGCAATGAC (SEQ ID NO: 15);

a DENV3 second probe comprising a sequence at least 90% identical to CACGGTGACACAGATGGCAATGAC (SEQ ID NO: 16);

a CHIKV forward primer comprising a sequence at least 90% identical to GGCGCCTACTGCTTCTGCGAC (SEQ ID NO: 18);

a CHIKV reverse primer comprising a sequence at least 90% identical to TTGGTAAAGGACGCGGAGCTTAGC (SEQ ID NO: 21);

a CHIKV first probe comprising a sequence at least 90% identical to AGCGAAGCACATGTGGAGAAGTCC (SEQ ID NO: 19);

a CHIKV second probe comprising a sequence at least 90% identical to AGCGAAGCACACGTGGAGAAGTCC (SEQ ID NO: 20);

a DENV2 forward primer comprising a sequence at least 90% identical to ACACAGATGGCAATGACAGACACG (SEQ ID NO: 22);

a DENV2 reverse primer comprising a sequence at least 90% identical to CCAAGGCTGCATTGCTTCTCAC (SEQ ID NO: 24);

a DENV2 probe comprising a sequence at least 90% identical to TGGAAAGAACTAGGAAAGAAAAAGACAC (SEQ ID NO: 23);

a DENV4 first forward primer comprising a sequence at least 90% identical to TGGTTAGACCACCTTTCAATATG (SEQ ID NO: 25);

a DENV4 second forward primer comprising a sequence at least 90% identical to TGGCTAGACCACCTTTCAATATG (SEQ ID NO: 26);

a DENV4 reverse primer comprising a sequence at least 90% identical to TGCTAGCACCATCCGTAA (SEQ ID NO: 28); and

a DENV4 probe comprising a sequence at least 90% identical to CCTCAAGGGTTGGTGAAGAGATTC (SEQ ID NO: 27).

In another aspect, there is provided a method for detecting and/or differentiating and/or quantifying virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, the method comprising: subjecting the sample to a reverse transcription polymerase chain reaction (RT-PCR) using primers and a probe specific for CHIKV E1 glycoprotein, primers and a probe specific for DENV1 Non Structural protein 5 (NS5), primers and a probe specific for DENV2 NS5, primers and a probe specific for DENV3 NS5, primers and a probe specific for DENV4 capsid, and primers and a probe specific for ZIKV NS5.

In some examples, the primers and probes comprise:

a ZIKV forward primer
comprising a sequence at least 90% identical to
CCTTGGATTCTTGAACGAGGATCAC
(SEQ ID NO: 7);
a ZIKV reverse primer
comprising a sequence at least 90% identical to
GCTTCATTCTCCAGATCAAACCTGC
(SEQ ID NO: 9)
or
GCTTCATTCTCTAGATCAAACCTGC
(SEQ ID NO: 32);
a ZIKV probe
comprising a sequence at least 90% identical to
TACCAGGAGGAAGGATGTATGCAG
(SEQ ID NO: 8)
or
ACCAGGAGGAAAGATGTACGCAG
(SEQ ID NO: 33);
a DENV1 first forward primer
comprising a sequence at least 90% identical to
GGCTGAAGAAAGTCACAGAAG
(SEQ ID NO: 10);
a DENV1 second forward primer
comprising a sequence at least 90% identical to
GGCTGAAGAAAGTCACTGAAG
(SEQ ID NO: 11);
a DENV1 reverse primer
comprising a sequence at least 90% identical to
GAGGACTCACCAATATCACACAA
(SEQ ID NO: 13);
a DENV1 probe
comprising a sequence at least 90% identical to
ACCTATGGATGGAACCTAGTAAAGCT
(SEQ ID NO: 12);
a DENV3 forward primer
comprising a sequence at least 90% identical to
GCTCAGCCTCCTCCATGATAAATG
(SEQ ID NO: 14);
a DENV3 reverse primer
comprising a sequence at least 90% identical to
GGGTGTCCTGGTGTCCACTTTCTC
(SEQ ID NO: 17);
a DENV3 first probe
comprising a sequence at least 90% identical to
CATGGTGACACAGATGGCAATGAC
(SEQ ID NO: 15);
a DENV3 second probe
comprising a sequence at least 90% identical to
CACGGTGACACAGATGGCAATGAC
(SEQ ID NO: 16);

a CHIKV forward primer comprising a sequence at least 90% identical to GGCGCCTACTGCTTCTGCGAC (SEQ ID NO: 18);

a CHIKV reverse primer comprising a sequence at least 90% identical to TTGGTAAAGGACGCGGAGCTTAGC (SEQ ID NO: 21);

a CHIKV first probe comprising a sequence at least 90% identical to AGCGAAGCACATGTGGAGAAGTCC (SEQ ID NO: 19);

a CHIKV second probe comprising a sequence at least 90% identical to AGCGAAGCACACGTGGAGAAGTCC (SEQ ID NO: 20);

a DENV2 forward primer comprising a sequence at least 90% identical to ACACAGATGGCAATGACAGACACG (SEQ ID NO: 22);

a DENV2 reverse primer comprising a sequence at least 90% identical to CCAAGGCTGCATTGCTTCTCAC (SEQ ID NO: 24);

a DENV2 probe comprising a sequence at least 90% identical to TGGAAAGAACTAGGAAAGAAAAAGACAC (SEQ ID NO: 23);

a DENV4 first forward primer comprising a sequence at least 90% identical to TGGTTAGACCACCTTTCAATATG (SEQ ID NO: 25);

a DENV4 second forward primer comprising a sequence at least 90% identical to TGGCTAGACCACCTTTCAATATG (SEQ ID NO: 26);

a DENV4 reverse primer comprising a sequence at least 90% identical to TGCTAGCACCATCCGTAA (SEQ ID NO: 28); and

a DENV4 probe comprising a sequence at least 90% identical to CCTCAAGGGTTGGTGAAGAGATTC (SEQ ID NO: 27).

In some examples, wherein:

the ZIKV forward primer comprising
(SEQ ID NO: 7)
CCTTGGATTCTTGAACGAGGATCAC;
the ZIKV reverse primer comprising
(SEQ ID NO: 9)
GCTTCATTCTCCAGATCAAACCTGC
or
(SEQ ID NO: 32)
GCTTCATTCTCTAGATCAAACCTGC;
the ZIKV probe comprising
(SEQ ID NO: 8)
TACCAGGAGGAAGGATGTATGCAG
or
(SEQ ID NO: 33)
ACCAGGAGGAAAGATGTACGCAG;
the DENV1 first forward primer comprising
(SEQ ID NO: 10)
GGCTGAAGAAAGTCACAGAAG;
the DENV1 second forward primer comprising
(SEQ ID NO: 11)
GGCTGAAGAAAGTCACTGAAG;
the DENV1 reverse primer comprising
(SEQ ID NO: 13)
GAGGACTCACCAATATCACACAA;
the DENV1 probe comprising
(SEQ ID NO: 12)
ACCTATGGATGGAACCTAGTAAAGCT;
the DENV3 forward primer comprising
(SEQ ID NO: 14)
GCTCAGCCTCCTCCATGATAAATG;
the DENV3 reverse primer comprising
(SEQ ID NO: 17)
GGGTGTCCTGGTGTCCACTTTCTC;
the DENV3 first probe comprising
(SEQ ID NO: 15)
CATGGTGACACAGATGGCAATGAC;
the DENV3 second probe comprising
(SEQ ID NO: 16)
CACGGTGACACAGATGGCAATGAC;
the CHIKV forward primer comprising
(SEQ ID NO: 18)
GGCGCCTACTGCTTCTGCGAC;
the CHIKV reverse primer comprising
(SEQ ID NO: 21)
TTGGTAAAGGACGCGGAGCTTAGC;
the CHIKV first probe comprising
(SEQ ID NO: 19)
AGCGAAGCACATGTGGAGAAGTCC;
the CHIKV second probe comprising
(SEQ ID NO: 20)
AGCGAAGCACACGTGGAGAAGTCC;
the DENV2 forward primer comprising
(SEQ ID NO: 22)
ACACAGATGGCAATGACAGACACG;
the DENV2 reverse primer comprising
(SEQ ID NO: 24)
CCAAGGCTGCATTGCTTCTCAC;
the DENV2 probe comprising
(SEQ ID NO: 23)
TGGAAAGAACTAGGAAAGAAAAAGACAC;
the DENV4 first forward primer comprising
(SEQ ID NO: 25)
TGGTTAGACCACCTTTCAATATG;
the DENV4 second forward primer comprising
(SEQ ID NO: 26)
TGGCTAGACCACCTTTCAATATG;
the DENV4 reverse primer comprising
(SEQ ID NO: 28)
TGCTAGCACCATCCGTAA;
and
the DENV4 probe comprising
(SEQ ID NO: 27)
CCTCAAGGGTTGGTGAAGAGATTC.

In some examples, the primers and probes comprise a detectable label.

In some examples, the detectable label comprises a fluorophore, a quencher, or a combination thereof.

In another aspect, there is provided a kit for detecting Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, comprising: an agent specific for detecting CHIKV E1 glycoprotein, an agent specific for detecting DENV1 Non Structural protein 5 (NS5), an agent specific for detecting DENV2 NS5, an agent specific for detecting DENV3 NS5, an agent specific for detecting DENV4 capsid, and an agent specific for detecting ZIKV NS5.

In some examples, the kit may comprise an agent for detecting a region or fragment thereof having 80% sequence identity to SEQ ID NO: 1; an agent for detecting a region or fragment thereof having 80% sequence identity to SEQ ID NO: 2; an agent for detecting a region or fragment thereof having 80% sequence identity to SEQ ID NO: 3; an agent for detecting a region or fragment thereof having 80% sequence identity to SEQ ID NO: 4; an agent for detecting a region or fragment thereof having 80% sequence identity to SEQ ID NO: 5; and an agent for detecting a region or fragment thereof having 80% sequence identity to SEQ ID NO: 6.

In some examples, the kit may comprise the agent that comprises primers and probes comprising:

a ZIKV forward primer comprising a sequence at least 90% identical to CCTTGGATTCTTGAACGAGGATCAC (SEQ ID NO: 7);

a ZIKV reverse primer comprising a sequence at least 90% identical to GCTTCATTCTCCAGATCAAACCTGC (SEQ ID NO: 9) or GCTTCATTCTCTAGATCAAACCTGC (SEQ ID NO: 32);

a ZIKV probe comprising a sequence at least 90% identical to TACCAGGAGGAAGGATGTATGCAG (SEQ ID NO: 8) or ACCAGGAGGAAAGATGTACGCAG (SEQ ID NO: 33);

a DENV1 first forward primer comprising a sequence at least 90% identical to GGCTGAAGAAAGTCACAGAAG (SEQ ID NO: 10);

a DENV1 second forward primer comprising a sequence at least 90% identical to GGCTGAAGAAAGTCACTGAAG (SEQ ID NO: 11);

a DENV1 reverse primer comprising a sequence at least 90% identical to GAGGACTCACCAATATCACACAA (SEQ ID NO: 13);

a DENV1 probe comprising a sequence at least 90% identical to ACCTATGGATGGAACCTAGTAAAGCT (SEQ ID NO: 12);

a DENV3 forward primer comprising a sequence at least 90% identical to GCTCAGCCTCCTCCATGATAAATG (SEQ ID NO: 14);

a DENV3 reverse primer comprising a sequence at least 90% identical to GGGTGTCCTGGTGTCCACTTTCTC (SEQ ID NO: 17);

a DENV3 first probe comprising a sequence at least 90% identical to CATGGTGACACAGATGGCAATGAC (SEQ ID NO: 15);

a DENV3 second probe comprising a sequence at least 90% identical to CACGGTGACACAGATGGCAATGAC (SEQ ID NO: 16);

a CHIKV forward primer comprising a sequence at least 90% identical to GGCGCCTACTGCTTCTGCGAC (SEQ ID NO: 18);

a CHIKV reverse primer comprising a sequence at least 90% identical to TTGGTAAAGGACGCGGAGCTTAGC (SEQ ID NO: 21);

a CHIKV first probe comprising a sequence at least 90% identical to AGCGAAGCACATGTGGAGAAGTCC (SEQ ID NO: 19);

a CHIKV second probe comprising a sequence at least 90% identical to AGCGAAGCACACGTGGAGAAGTCC (SEQ ID NO: 20);

a DENV2 forward primer comprising a sequence at least 90% identical to ACACAGATGGCAATGACAGACACG (SEQ ID NO: 22);

a DENV2 reverse primer comprising a sequence at least 90% identical to CCAAGGCTGCATTGCTTCTCAC (SEQ ID NO: 24);

a DENV2 probe comprising a sequence at least 90% identical to TGGAAAGAACTAGGAAAGAAAAAGACAC (SEQ ID NO: 23);

a DENV4 first forward primer comprising a sequence at least 90% identical to TGGTTAGACCACCTTTCAATATG (SEQ ID NO: 25);

a DENV4 second forward primer comprising a sequence at least 90% identical to TGGCTAGACCACCTTTCAATATG (SEQ ID NO: 26);

a DENV4 reverse primer comprising a sequence at least 90% identical to TGCTAGCACCATCCGTAA (SEQ ID NO: 28); and

a DENV4 probe comprising a sequence at least 90% identical to CCTCAAGGGTTGGTGAAGAGATTC (SEQ ID NO: 27).

### BRIEF DESCRIPTION OF THE DRAWINGS

Exemplary embodiments of the invention will be better understood and readily apparent to one of ordinary skill in the art from the following written description, by way of example only, and in conjunction with the drawings, in which:

FIG. 1 shows exemplary graphs showing the stages of PCR amplification plot in linear (FIG. 1A) and log views (FIG. 1B).

FIG. 2 shows exemplary graphs showing false positive curves.

FIG. 3 shows amplification plot of a sample with a “wandering” curve (FIG. 3A) and the corresponding background fluorescence view (FIG. 3B).

FIG. 4 illustrates the PCR efficiencies of single-plex conditions when detecting CHIKV using SIgN-DXD PCR set 1 (FIG. 4A), SIgN-DXD PCR set 2 (FIG. 4B), and CDC PCR (FIG. 4C).

FIG. 5 illustrates the PCR efficiencies of single-plex conditions when detecting DENV1 using SIgN-DXD PCR set 1 (FIG. 5A) and CDC PCR (FIG. 5B).

FIG. 6 illustrates the PCR efficiencies of single-plex conditions when detecting DENV2 using SIgN-DXD PCR set 1 (FIG. 6A), SIgN-DXD PCR set 2 (FIG. 6B), and SIgN-DXD PCR set 4 (FIG. 6C) and CDC PCR (FIG. 6D).

FIG. 7 illustrates the PCR efficiencies of single-plex conditions when detecting DENV3 using SIgN-DXD PCR set 1 (FIG. 7A), SIgN-DXD PCR set 2 (FIG. 7B), SIgN-DXD PCR set 3 (FIG. 7C) and SIgN-DXD PCR set 4 (FIG. 7D) and CDC PCR (FIG. 7E).

FIG. 8 illustrates the PCR efficiencies of single-plex conditions when detecting DENV4 using SIgN-DXD PCR set 2 (FIG. 8A), SIgN-DXD PCR set 3 (FIG. 8B), SIgN-DXD PCR set 4 (FIG. 8C) and CDC PCR (FIG. 8D).

FIG. 9 illustrates the PCR efficiencies of single-plex conditions when detecting ZIKV using SIgN-DXD PCR set 1 (FIG. 9A) and CDC PCR (FIG. 9B).

FIG. 10 illustrates the limit of detection (95% LLOD) of the multiplex PCR in detecting ZIKV.

FIG. 11 illustrates the limit of detection (95% LLOD) of the multiplex PCR in detecting DENV1.

FIG. 12 illustrates the limit of detection (95% LLOD) of the multiplex PCR in detecting DENV3.

FIG. 13 illustrates the limit of detection (95% LLOD) of the multiplex PCR in detecting CHIKV.

FIG. 14 illustrates the limit of detection (95% LLOD) of the multiplex PCR in detecting DENV2.

FIG. 15 illustrates the limit of detection (95% LLOD) of the multiplex PCR in detecting DENV4.

FIG. 16 illustrates the specificity of ZIKV (FIG. 16A), DENV1 (FIG. 16B), DENV3 (FIG. 16C), CHIKV (FIG. 16D), DENV2 (FIG. 16E), DENV4 (FIG. 16F), SLEV (FIG. 16G), WNV (FIG. 16H), and YFV (FIG. 161) primer and probe sets.

FIG. 17 shows the detection of ZIKV viral load (FIG. 17A left panel) and no cross reactivity in other channels or mix (FIG. 17A right panel using Mix 1 and FIG. 17B using Mix 2).

FIG. 18 shows the detection of DENV1 (FIG. 18A left panel) and no cross reactivity in other channels or mix (FIG. 18A right panel using Mix 1 and FIG. 18B using Mix 2).

FIG. 19 shows the detection of DENV3 (FIG. 19A left panel) and no cross reactivity in other channels or mix (FIG. 19A right panel using Mix 1 and FIG. 19B using Mix 2).

FIG. 20 shows the detection of CHIKV (FIG. 20A left panel) and no cross reactivity in other channels or mix (FIG. 20A right panel using Mix 1 and FIG. 20B using Mix 2).

FIG. 21 shows the detection of DENV2 (FIG. 21A left panel) and no cross reactivity in other channels or mix (FIG. 21A right panel using Mix 1 and FIG. 21 B using Mix 2).

FIG. 22 shows the detection of DENV4 (FIG. 22A left panel) and no cross reactivity in other channels or mix (FIG. 22A right panel using Mix 1 and FIG. 22B using Mix 2).

FIG. 23 shows CHIKV E1 glycoprotein consensus sequence SEQ ID NO: 1.

FIG. 24 shows DENV1 Non-Structural protein 5 (NS5) consensus sequence SEQ ID NO: 2.

FIG. 25 shows DENV2 NS5 consensus sequence SEQ ID NO: 3.

FIG. 26 shows DENV3 NS5 consensus sequence SEQ ID NO: 4.

FIG. 27 shows DENV4 capsid consensus sequence SEQ ID NO: 5.

FIG. 28 shows ZIKV NS5 consensus sequence SEQ ID NO: 6.

### DETAILED DESCRIPTION

Chikungunya, Zika, and Dengue are three prevalent mosquito-borne viruses that cause similar disease symptoms. Distinguishing the causative virus in an infection is essential for appropriate treatment and care. The inventors of the present disclosure have developed a multiplex molecular diagnostic test that can differentially detect Chikungunya, the various serotypes of Dengue and Zika viruses.

In one aspect, there is provided a method of simultaneously detecting, differentiating, and/or quantifying Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, wherein the method comprising: determining the presence of the target regions or fragments thereof selected from the group consisting of Non Structural protein 5 (NS5) of Zika virus, NS5 of DENV1, NS5 of DENV2, NS5 of DENV3, Capsid of DENV4, and E1 glycoprotein of CHIKV.

In some examples, the method is to simultaneously detect three or more viruses. That is, in some examples, there is provided a method of simultaneously detecting, differentiating, and/or quantifying three or more virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, wherein the method comprising:

determining the presence of three or more target regions or fragments thereof selected from the group consisting of Non-Structural protein 5 (NS5) of ZIKV, NS5 of DENV1, NS5 of DENV2, NS5 of DENV3, Capsid of DENV4, and E1 glycoprotein of CHIKV.

In some examples, the method is to detect one or more viruses. That is, in some examples, there is provided a method of detecting, differentiating, and/or quantifying one or more virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, wherein the method comprising:

determining the presence of one or more target regions or fragments thereof selected from the group consisting of Non-Structural protein 5 (NS5) of ZIKV, NS5 of DENV1, NS5 of DENV2, NS5 of DENV3, Capsid of DENV4, and E1 glycoprotein of CHIKV.

As used herein, the term “simultaneously” refers to concurrent (synchronous or happening at the same time) detection/differentiating and/or quantification of the targets of interest. At the same time, the term “detect”, “detecting”, or “detection” refers to the discovering, distinguishing, or determining the presence of the target of interest or fragment thereof. Thus, the method as described herein allows for one sample to be used concurrently on whether the sample comprises any one of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV). Thus, in some examples, the method is capable of detecting three or more viruses, or four or more viruses, or five or more viruses, or all six viruses, which includes Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV).

As used herein, the term “target region” refers to a region or structure of the virus of interest that is to be analysed and/or detected. In some examples, the target region may refer to a target sequence that is a region of a nucleic acid that is to be analysed and comprises the sequence of the virus of interest.

As used herein, the terms “nucleic acid”, “oligonucleotide” and “polynucleotide” refer to primers, probes, and oligomer fragments. The terms are not limited by length and are generic to polymers (typically linear) of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA. Oligonucleotides of the present disclosure may be used as primers and/or probes. A nucleic acid or oligonucleotide may comprise the five biologically occurring bases (adenine, guanine, thymine, cystosine, and uracil) and/or bases other than the five biologically occurring bases. These bases may serve a number of purposes, e.g. to stabilize or destabilize hybridization; to promote or inhibit probe degradation; or as attachment points for detectable label (or moieties) or quencher. Included in the terms “nucleic acid”, “oligonucleotide” and “polynucleotide” may include complementary sequences thereof.

The selection of target regions as described herein surprisingly allows for the detection and differentiation of six viruses simultaneously. In particular, the target regions of the present disclosure allow for the distinction of the four DENV serotypes, ZIKV, and CHIKV to be performed concurrently. As shown in the Experimental section below, the target regions of the present disclosure are highly specific for each of the target of interest such that no cross-reactivity or non-specific result was observed. The selection of target regions could surprisingly allow for the individual detection of each of the four DENV serotypes, which is important as there is an increase risk of severe clinical manifestation in patients/subjects infected with subsequent/other serotypes.

The E1 glycoprotein of chikungunya virus (CHIKV) as disclosed herein may comprise a sequence or part of a sequence having at least 80% identity to SEQ ID NO: 1 or its fragment thereof. The E1 glycoprotein of chikungunya virus of SEQ ID NO: 1 is a consensus sequence of chikungunya virus that were obtained between 2005 to 2016 from various regions around the world. Detail of virus strains used to build the consensus sequence can be found in Table 5. In some examples, the target regions or fragments thereof may have at least 85%, or at least 90%, or at least 95%, or at least 96%, or at least 97%, or at least 98%, or at least 99%, or at least 100% sequence identity to SEQ ID NO: 1 or part of the sequence or its fragment thereof.

The Non-Structural protein 5 (NS5) of DENV1 (Dengue virus serotype 1) as disclosed herein may comprise a sequence or part of a sequence having at least 80% identity to SEQ ID NO: 2 or its fragment thereof. The NS5 of DENV1 of SEQ ID NO: 2 is a consensus sequence of DENV1 that were obtained from various regions around the world. Detail of virus strains used to build the consensus sequence can be found in Table 6. In some examples, the target regions or fragments thereof may have at least 85%, or at least 90%, or at least 95%, or at least 96%, or at least 97%, or at least 98%, or at least 99%, or at least 100% sequence identity to SEQ ID NO: 2 or part of the sequence or its fragment thereof.

The Non-Structural protein 5 (NS5) of DENV2 (Dengue virus serotype 2) as disclosed herein may comprise a sequence or part of a sequence having at least 80% identity to SEQ ID NO: 3 or its fragment thereof. The NS5 of DENV2 of SEQ ID NO: 3 is a consensus sequence of DENV2 that were obtained from various regions around the world. Detail of virus strains used to build the consensus sequence can be found in Table 7. In some examples, the target regions or fragments thereof may have at least 85%, or at least 90%, or at least 95%, or at least 96%, or at least 97%, or at least 98%, or at least 99%, or at least 100% sequence identity to SEQ ID NO: 3 or part of the sequence or its fragment thereof.

The Non-Structural protein 5 (NS5) of DENV3 (Dengue virus serotype 3) as disclosed herein may comprise a sequence or part of a sequence having at least 80% identity to SEQ ID NO: 4 or its fragment thereof. The NS5 of DENV3 of SEQ ID NO: 4 is a consensus sequence of DENV3 that were obtained from various regions around the world. Detail of virus strains used to build the consensus sequence can be found in Table 8. In some examples, the target regions or fragments thereof may have at least 85%, or at least 90%, or at least 95%, or at least 96%, or at least 97%, or at least 98%, or at least 99%, or at least 100% sequence identity to SEQ ID NO: 4 or part of the sequence or its fragment thereof.

The capsid of DENV4 (Dengue virus serotype 4) as disclosed herein may comprise a sequence or part of a sequence having at least 80% identity to SEQ ID NO: 5 or its fragment thereof. The capsid of DENV4 of SEQ ID NO: 5 is a consensus sequence of DENV4 that were obtained from various regions around the world. Detail of virus strains used to build the consensus sequence can be found in Table 9. In some examples, the target regions or fragments thereof may have at least 85%, or at least 90%, or at least 95%, or at least 96%, or at least 97%, or at least 98%, or at least 99%, or at least 100% sequence identity to SEQ ID NO: 5 or part of the sequence or its fragment thereof.

The NS5 of Zika virus (ZIKV) as disclosed herein may comprise a sequence or part of a sequence having at least 80% identity to SEQ ID NO: 6 or its fragment thereof. The NS5 of ZIKV of SEQ ID NO: 6 is a consensus sequence of ZIKV that were obtained from various regions around the world. Detail of virus strains used to build the consensus sequence can be found in Table 10. In some examples, the target regions or fragments thereof may have at least 85%, or at least 90%, or at least 95%, or at least 96%, or at least 97%, or at least 98%, or at least 99%, or at least 100% sequence identity to SEQ ID NO: 6 or part of the sequence or its fragment thereof.

In some examples, the target regions or fragments thereof are encoded by the sequence SEQ ID NO: 1 (CHIKV E1 consensus sequence); SEQ ID NO: 2 (DENV1 NS5 48+22 seq consensus sequence); SEQ ID NO: 3 (DENV2 NS5 consensus sequence); SEQ ID NO: 4 (DENV3 NS5 consensus sequence); SEQ ID NO: 5 (DENV4 Capsid consensus sequence); and SEQ ID NO: 6 (ZIKV NS5 check_56seq consensus sequence).

In some examples, the CHIKV E1
consensus sequence is
(SEQ ID NO: 1)
TACGAACACGTAACAGTGATCCCGAACACGGTGGG
AGTACCGTATAAGACTCTAGTCAACAGACCGGGCT
ACAGCCCCATGGTATTGGAGATGGAACTACTGTCA
GTCACTTTGGAGCCAACACTATCGCTTGATTACAT
CACGTGCGAGTACAAAACCGTCATCCCGTCTCCGT
ACGTGAAATGCTGCGGTACAGCAGAGTGCAAGGAC
AAAAACCTACCTGACTACAGCTGTAAGGTCTTCAC
CGGCGTCTACCCATTTATGTGGGGCGGCGCCTACT
GCTTCTGCGACGCTGAAAATACGCAATTGAGCGAA
GCACATGTGGAGAAGTCCGAATCATGCAAAACAGA
ATTTGCATCAGCATACAGGGCTCATACCGCATCCG
CATCAGCTAAGCTCCGCGTCCTTTACCAAGGAAAT
AACATCACTGTAACTGCCTATGCAAACGGCGACCA
TGCCGTCACAGTTAAGGACGCCAAATTCATTGTGG
GGCCAATGTCTTCAGCCTGGACACCTTTCGACAAC
AAAATTGTGGTGTACAAAGGTGACGTCTATAACAT
GGACTACCCGCCCTTTGGCGCAGGAAGACCAGGAC
AATTTGGCGATATCCAAAGTCGCACACCTGAGAGT
AAAGACGTCTATGCTAATACACAACTGGTACTGCA
GAGACCGGCTGCGGGTACGGTACACGTGCCATACT
CTCAGGCACCATCTGGCTTTAAGTATTGGCTAAAA
GAACGAGGGGCGTCGCTGCAGCACACAGCACCATT
TGGCTGCCAAATAGCAACAAACCCGGTAAGAGCGG
TGAACTGCGCCGTAGGGAACATGCCCATCTCCATC
GACATACCGGAAGCGGCCTTCACTAGGGTCGTCGA
CGCGCCCTCTTTAACGGACATGTCGTGCGAGGTAC
CAGCCTGCACCCATTCCTCAGACTTTGGGGGCGTC
GCCATTATTAAATATGCAGCCAGCAAGAAAGGCAA
GTGTGCGGTGCATTCGATGACTAACGCCGTCACTA
TTCGGGAAGCTGAGATAGAAGTTGAAGGGAATTCT
CAGCTGCAAATCTCTTTCTCGACGGCCTTAGCCAG
CGCCGAATTCCGCGTACAAGTCTGTTCTACACAAG
TACACTGTGCAGCCGAGTGCCACCCCCCGAAGGAC
CACATAGTCAACTACCCGGCGTCACATACCACCCT
CGGGGTCCAGGACATTTCCGCTACGGCGATGTCAT
GGGTGCAGAAGATCACGGGAGGTGTGGGACTGGTT
GTCGCTGTTGCAGCACTGATTCTAATCGTGGTGCT
ATGCGTGTCGTTCAGCAGGCAC.
In some examples, the DENV1 NS5
consensus sequence is
(SEQ ID NO: 2)
GGCACGGGAGCCCAAGGGGAAACACTGGGAGAGAA
ATGGAAAAGACAGCTGAACCAACTGAGCAAGTCAG
AATTCAACACCTACAAAAGGAGTGGGATTATGGAG
GTGGACAGATCCGAAGCCAAAGAGGGACTGAAAAG
AGGAGAAACAACCAAACATGCAGTGTCGAGAGGAA
CCGCCAAACTGAGGTGGTTTGTGGAGAGGAACCTT
GTGAAACCAGAAGGGAAAGTCATAGACCTCGGTTG
TGGAAGAGGTGGCTGGTCATATTATTGCGCTGGGC
TGAAGAAAGTCACAGAAGTGAAGGGATACACAAAA
GGAGGACCTGGACATGAGGAACCAATCCCAATGGC
GACCTATGGATGGAACCTAGTAAAGCTACACTCCG
GGAAAGATGTATTCTTTATACCACCTGAGAAATGT
GACACCCTTTTGTGTGATATTGGTGAGTCCTCTCC
GAACCCAACTATAGAAGAAGGAAGAACGTTACGTG
TTCTAAAGATGGTGGAACCATGGCTCAGAGGAAAC
CAATTTTGCATAAAAATTCTAAATCCCTACATGCC
AAGTGTGGTAGAAACTCTGGAGCAAATGCAAAGAA
AACATGGAGGAATGCTAGTGCGAAATCCACTCTCA
AGAAATTCCACTCATGAAATGTACTGGGTTTCATG
TGGAACAGGAAACATTGTGTCAGCAGTAAACATGA
CATCCAGAATGTTGCTAAATCGATTCACAATGGCT
CACAGGAAGCCAACATATGAAAGAGACGTGGACTT
AGGCGCTGGAACAAGACATGTGGCAGTGGAACCAG
AGGTAGCCAACCTAGATATCATTGGCCAGAGGATA
GAGAACATAAAAAATGAACACAAGTCAACATGGCA
TTATGATGAGGACAATCCATACAAAACATGGGCCT
ATCATGGATCATATGAGGTCAAGCCATCAGGATCA
GCCTCATCCATGGTCAATGGTGTGGTGAGACTGCT
CACCAAACCATGGGATGTCATCCCCATGGTCACAC
AAATAGCCATGACTGACACCACACCCTTTGGACAA
CAGAGGGTGTTTAAAGAGAAAGTTGACACGCGCAC
ACCAAAAGCAAAACGAGGCACAGCACAAATCATGG
AGGTGACAGCCAAGTGGTTATGGGGTTTTCTTTCT
AGAAACAAAAAACCCAGAATCTGCACAAGAGAGGA
GTTCACAAGAAAAGTTAGGTCAAACGCAGCCATTG
GAGCAGTGTTCGTTGATGAAAATCAATGGAACTCA
GCAAAAGAAGCAGTGGAAGATGAACGGTTCTGGGA
CCTTGTGCACAGAGAGAGGGAGCTTCATAAACAGG
GAAAATGTGCCACGTGTGTCTACAACATGATGGGG
AAGAGAGAGAAAAAACTAGGAGAGTTTGGAAAGGC
AAAAGGAAGTCGTGCAATATGGTACATGTGGTTGG
GAGCACGCTTTCTAGAGTTCGAAGCCCTTGGTTTC
ATGAATGAAGATCACTGGTTCAGCAGAGAGAATTC
ACTCAGTGGAGTGGAAGGAGAAGGACTCCACAAAC
TTGGATACATACTCAGAGACATATCAAAGATTCCA
GGGGGAAATATGTATGCAGATGACACAGCCGGATG
GGACACAAGAATAACAGAGGATGATCTTCAGAATG
AGGCCAAAATCACTGACATCATGGAACCTGAACAT
GCCCTACTGGCTACGTCAATCTTTAAGCTAACCTA
CCAAAATAAGGTGGTAAGGGTGCAGAGACCAGCAA
AAAATGGAACCGTGATGGATGTCATATCCAGACGT
GACCAGAGAGGAAGTGGACAGGTCGGAACTTATGG
CTTAAACACTTTCACCAACATGGAGGCCCAACTAA
TAAGACAAATGGAGTCTGAGGGAATCTTTTCACCC
AGCGAATTGGAAACCCCAAATTTAGCCGAGAGAGT
TCTCGACTGGTTGGAAAAACATGGCGTCGAAAGGC
TGAAAAGAATGGCAATCAGCGGAGATGACTGCGTG
GTGAAACCAATTGATGACAGGTTCGCAACAGCCTT
AACAGCTCTGAATGACATGGGAAAAGTAAGAAAAG
ACATACCGCAATGGGAACCTTCAAAAGGATGGAAT
GATTGGCAACAAGTGCCTTTCTGTTCACACCATTT
CCACCAGCTGATTATGAAGGATGGGAGGGAAATAG
TGGTGCCATGCCGCAACCAAGATGAACTTGTGGGT
AGGGCTAGAGTATCACAAGGCGCCGGATGGAGCCT
GAGAGAAACTGCATGCCTAGGCAAGTCATATGCAC
AAATGTGGCAGCTGATGTACTTCCACAGGAGAGAC
CTGAGACTAGCGGCTAATGCTATCTGTTCAGCCGT
TCCAGTTGATTGGGTCCCAACCAGCCGCACCACCT
GGTCGATCCATGCCCACCACCAATGGATGACAACA
GAAGACATGTTGTCAGTGTGGAATAGGGTTTGGAT
AGAGGAAAACCCATGGATGGAGGACAAAACTCATG
TATCCAGTTGGGAAGATGTTCCATACCTAGGGAAA
AGGGAAGATCAATGGTGTGGATCCCTGATAGGCTT
AACAGCAAGGGCCACCTGGGCCACCAACATACAAG
TGGCCATAAACCAAGTGAGAAGGCTCATTGGGAAT
GAGAATTATCTAGATTACATGACATCAATGAAGAG
ATTCAAGAACGAGAGTGATCCCGAAGGGGCACTCT
GG.
In some examples, the DENV2 NS5
consensus sequence is
(SEQ ID NO: 3)
GGAACTGGCAACATAGGAGAGACACTTGGAGAAAA
ATGGAAAAGCCGATTAAACGCACTGGGAAAAAGTG
AATTTCAGATCTACAAGAAAAGTGGAATCCAGGAA
GTGGATAGAACCTTAGCAAAAGAAGGCATCAAAAG
AGGAGAAACGGACCACCACGCTGTGTCGCGAGGCT
CAGCAAAACTGAGATGGTTCGTCGAGAGAAATATG
GTCACACCAGAAGGGAAGGTGGTGGACCTCGGTTG
CGGCAGAGGGGGCTGGTCATACTATTGTGGGGGAC
TAAAGAATGTAAGAGAAGTCAAAGGCCTAACAAAA
GGAGGACCAGGACACGAAGAACCCATCCCCATGTC
AACATATGGGTGGAATCTAGTGCGTCTGCAAAGTG
GAGTTGACGTTTTCTTCACCCCGCCAGAAAAGTGT
GATACATTGTTGTGTGACATAGGGGAGTCGTCACC
AAATCCCACGATAGAAGCAGGACGAACACTCAGAG
TCCTCAACTTAGTGGAAAATTGGTTGAACAATAAC
ACCCAATTTTGCATAAAGGTTCTCAACCCATATAT
GCCCTCAGTCATAGAAAAAATGGAAACACTACAAA
GGAAATATGGAGGAGCCTTAGTGAGGAATCCACTC
TCACGAAACTCCACACATGAGATGTACTGGGTATC
CAATGCTACCGGGAACATAGTGTCATCAGTGAACA
TGATTTCAAGGATGTTGATTAACAGATTCACAATG
AAACACAAGAAAGCCACCTACGAGCCAGATGTTGA
CCTAGGAAGTGGAACCCGCAACATTGGAATTGAAA
GTGAGATACCAAATCTAGACATAATAGGAAAGAGA
ATAGAGAAAATAAAACAAGAGCATGAAACATCATG
GCACTATGACCAAGACCACCCATACAAAACGTGGG
CTTACCATGGCAGCTATGAAACAAAACAAACTGGA
TCAGCATCATCTATGGTGAACGGAGTGGTCAGACT
GCTGACAAAACCTTGGGACGTCGTCCCTATGGTGA
CACAGATGGCAATGACAGACACGACTCCATTTGGA
CAACAGCGCGTTTTCAAAGAGAAAGTGGACACGAG
AACCCAAGAACCGAAGGAAGGCACAAAGAAACTGA
TGAAAATCACGGCAGAGTGGCTTTGGAAAGAACTA
GGAAAGAAAAAGACACCTAGGATGTGTACCAGAGA
AGAATTCACAAGAAAGGTGAGAAGCAATGCAGCCT
TGGGGGCCATATTCACTGATGAGAACAAATGGAAA
TCGGCACGTGAGGCTGTTGAAGATAGTAGGTTTTG
GGAGCTGGTTGACAGGGAAAGAAATCTCCATCTTG
AAGGAAAGTGTGAAACATGTGTGTACAACATGATG
GGAAAAAGAGAGAAGAAACTAGGGGAGTTCGGCAA
GGCAAAAGGTAGCAGAGCCATATGGTACATGTGGC
TTGGAGCACGCTTCTTAGAGTTTGAAGCCCTAGGA
TTCTTGAATGAAGATCACTGGTTCTCCAGAGGGAA
CTCCCTGAGTGGAGTGGAAGGAGAAGGGCTGCACA
GGCTAGGCTACATTTTAAGAGACGTGAGCAAGAAG
GAAGGGGGAGCAATGTACGCCGATGATACAGCAGG
ATGGGACACAAGAATCACACTAGAAGACTTAAAAA
ATGAAGAAATGGTAACAAACCACATGAAAGGAGAA
CACAAGAAACTAGCCGAGGCCATATTCAAATTAAC
GTACCAAAACAAGGTGGTGCGTGTGCAAAGACCAA
CACCAAGAGGCACAGTAATGGATATCATATCGAGA
AGAGACCAAAGAGGCAGTGGGCAAGTCGGCACCTA
TGGCCTTAATACTTTCACCAATATGGAAGCCCAAT
TAATTAGACAGATGGAGGGAGAAGGAATCTTCAAA
AGCATTCAGCAGCATTCAGCACCTGACAGTCACAG
AAGAAATCGCTGTACAGAACTGGTTAGCAAGAGTG
GGGCGTGAAAGGCTATCAAGAATGGCCATCAGTGG
AGATGATTGTGTTGTAAAACCTTTAGATGACAGAT
TTGCAAGTGCTTTAACAGCTCTAAATGACATGGGA
AAAGTTAGGAAAGATATACAACAATGGGAACCTTC
AAGAGGATGGAACGATTGGACACAAGTGCCTTTCT
GTTCACACCATTTTCATGAGTTAGTCATGAAAGAT
GGTCGCGTGCTCGTAGTCCCATGCAGAAACCAAGA
TGAACTGATTGGTAGAGCCCGAATTTCCCAGGGAG
CCGGGTGGTCTTTGAAGGAGACGGCCTGTTTGGGG
AAGTCTTACGCCCAAATGTGGACCCTGATGTACTT
CCACAGACGTGACCTCAGACTGGCGGCAAATGCCA
TTTGCTCGGCAGTCCCGTCACATTGGGTTCCAACA
AGTCGAACAACCTGGTCCATACACGCTAAGCATGA
ATGGATGACGACGGAAGACATGCTGGCAGTCTGGA
ACAGGGTGTGGATCCAAGAAAACCCGTGGATGGAA
GACAAAACTCCAGTGGAATCATGGGAAGAAGTCCC
ATACTTGGGGAAAAGAGAAGACCAATGGTGCGGCT
CATTGATTGGGCTAACAAGCAGGGCTACCTGGGCA
AAGAACATCCAAACAGCAATAAATCAAGTCAGATC
CCTTATAGGCAATGAGGAATACACAGACTACATGC
CATCCATGAAGAGATTCAGAAGGGAAGAGGAAGAG
GCAGGTGTCCTGTGG.
In some examples, the DENV3 NS5
consensus sequence is
(SEQ ID NO: 4)
GGAACAGGCTCACAAGGTGAAACTTTAGGAGAAAA
ATGGAAAAAGAAATTAAATCAATTATCCCGGAAAG
AGTTTGACCTTTACAAGAAATCTGGAATCACTGAA
GTGGATAGAACAGAAGCCAAAGAAGGGTTGAAAAG
AGGAGAAATAACACATCATGCCGTGTCCAGAGGTA
GCGCAAAACTTCAATGGTTTGTGGAGAGAAACATG
GTCATTCCCGAAGGAAGAGTCATAGACTTGGGCTG
TGGAAGAGGAGGCTGGTCATATTACTGTGCAGGAC
TGAAAAAAGTCACAGAAGTGCGAGGATACACAAAA
GGCGGTCCAGGACACGAAGAACCAGTACCTATGTC
CACATATGGATGGAACATAGTTAAGTTAATGAGTG
GAAAGGATGTGTTTTATCTTCCACCTGAAAAGTGT
GACACCCTGTTGTGTGACATTGGAGAATCTTCACC
AAGCCCAACAGTGGAAGAAAGCAGAACTATAAGAG
TTTTGAAGATGGTTGAACCATGGCTAAAAAACAAC
CAGTTTTGCATTAAAGTATTGAACCCTTACATGCC
AACTGTGATTGAGCACCTAGAAAGACTACAAAGGA
AACATGGAGGAATGCTTGTGAGAAATCCACTTTCA
CGAAACTCCACGCACGAAATGTACTGGATATCTAA
TGGCACAGGTAACATTGTCTCTTCAGTCAACATGG
TATCTAGACTGCTACTGAACAGGTTCACGATGACA
CACAGAAGACCCACCATAGAGAAAGATGTGGATTT
AGGAGCAGGAACTCGACATGTTAATGCGGAACCAG
AAACACCCAACATGGATGTCATTGGGGAAAGAATA
AAAAGGATCAAGGAGGAGCATAATTCAACATGGCA
CTATGATGACGAAAACCCCTACAAAACGTGGGCTT
ACCATGGATCTTATGAAGTCAAAGCCACAGGCTCA
GCCTCCTCCATGATAAATGGAGTCGTGAAACTCCT
CACTAAACCATGGGATGTGGTGCCCATGGTGACAC
AGATGGCAATGACAGATACAACTCCATTTGGCCAG
CAGAGAGTCTTTAAAGAGAAAGTGGACACCAGGAC
ACCCAGGCCCATGCCAGGAACAAGAAAGGTTATGG
AGATCACAGCGGAGTGGCTCTGGAGAACCCTGGGA
AGGAACAAAAAACCCAGGTTATGCACAAGGGAAGA
GTTTACAAAAAAGGTCAGAACTAACGCAGCCATGG
GCGCCGTTTTCACAGAGGAGAACCAATGGGACAGC
GCGAAAGCTGCTGTTGAGGATGAGGATTTTTGGAA
ACTTGTGGACAGAGAACGTGAACTCCACAAATTGG
GCAAGTGTGGAAGCTGTGTTTACAACATGATGGGC
AAGAGAGAGAAGAAACTTGGAGAGTTTGGCAAAGC
AAAAGGCAGTAGAGCTATATGGTACATGTGGTTGG
GAGCCAGGTACCTTGAGTTCGAAGCCCTTGGATTC
TTAAATGAAGACCACTGGTTCTCGCGTGAGAACTC
TTACAGTGGAGTAGAAGGAGAAGGACTGCACAAGC
TAGGCTATATATTAAGGGACATTTCCAAGATACCC
GGAGGAGCTATGTATGCTGATGACACAGCTGGTTG
GGACACAAGAATAACAGAAGATGACCTGCACAATG
AGGAAAAGATCACACAGCAAATGGACCCTGAACAC
AGGCAGTTAGCGAACGCTATATTTAAGCTCACATA
CCAAAACAAAGTGGTCAAAGTTCAACGACCGACTC
CAACAGGCACGGTAATGGACATCATATCTAGGAAA
GACCAAAGAGGCAGTGGACAGGTGGGAACTTATGG
TCTGAATACATTCACCAACATGGAAGCCCAGTTAA
TCAGACAAATGGAAGGAGAAGGTGTGCTGTCAAAG
GCAGACCTCGGCAGACCTCGAGAACCCTCATCTGC
CAGAGAAGAAAATTACACAATGGTTGGAAACCAAA
GGAGTGGAGAGGTTAAAAAGAATGGCCATTAGCGG
GGATGATTGCGTAGTGAAACCAATCGATGACAGGT
TCGCTAATGCCCTGCTTGCTCTGAACGATATGGGA
AAGGTTCGGAAAGACATACCTCAATGGCAGCCATC
AAAGGGATGGCATGATTGGCAACAGGTTCCTTTCT
GCTCCCACCACTTTCATGAATTGATCATGAAAGAT
GGAAGAAAGTTGGTGGTTCCCTGCAGACCCCAGGA
CGAACTAATAGGAAGAGCAAGAATCTCTCAAGGAG
CGGGATGGAGCCTTAGAGAAACCGCATGTCTGGGG
AAAGCCTACGCTCAAATGTGGAGTCTCATGTATTT
TCACAGAAGAGATCTCAGACTAGCATCCAACGCCA
TATGTTCAGCAGTACCAGTCCACTGGGTCCCCACA
AGTAGAACGACATGGTCTATTCATGCTCACCATCA
GTGGATGACCACAGAAGACATGCTTACTGTCTGGA
ACAGGGTGTGGATCGAGGACAATCCATGGATGGAA
GACAAAACTCCAGTCACAACCTGGGAAAATGTTCC
ATATCTAGGGAAGAGAGAAGACCAATGGTGCGGAT
CACTTATTGGTCTCACTTCCAGAGCAACCTGGGCC
CAGAACATACCCACAGCAATTCAACAGGTGAGAAG
CCTTATAGGCAATGAAGAGTTTCTGGACTACATGC
CTTCAATGAAGAGATTCAGGAAGGAGGAGGAGTCG
GAGGGAGCCATTTGG.
In some examples, the DENV4 caspid
consensus sequence is
(SEQ ID NO: 5)
ATGAACCAACGAAAAAAGGTGGTTAGACCACCTTT
CAATATGCTGAAACGCGAGAGAAACCGCGTATCAA
CCCCTCAAGGGTTGGTGAAGAGATTCTCAACCGGA
CTTTTTTCTGGGAAAGGACCCTTACGGATGGTGCT
AGCATTCATCACGTTTTTGCGAGTCCTTTCCATCC
CACCAACAGCAGGGATTCTGAAGAGATGGGGACAG
TTGAAGAAAAATAAGGCCATCAAGATACTGATTGG
ATTCAGGAAGGAGATAGGCCGCATGCTGAACATCT
TGAACGGGAGAAAAAGGTCAACGATAACATTGCTG
TGCTTGATTCCCACCGTAATGGCG.
In some examples, the ZIKV NS5
consensues sequence is
(SEQ ID NO: 6)
GGGGGTGGAACAGGAGAGACCCTGGGAGAGAAATG
GAAGGCCCGCTTGAACCAGATGTCGGCCCTGGAGT
TCTACTCCTACAAAAAGTCAGGCATCACCGAGGTG
TGCAGAGAAGAGGCCCGCCGCGCCCTCAAGGACGG
TGTGGCAACGGGAGGCCATGCTGTGTCCCGAGGAA
GTGCAAAGCTGAGATGGTTGGTGGAGCGGGGATAC
CTGCAGCCCTATGGAAAGGTCATTGATCTTGGATG
TGGCAGAGGGGGCTGGAGTTACTACGCCGCCACCA
TCCGCAAAGTTCAAGAAGTGAAAGGATACACAAAA
GGAGGCCCTGGTCATGAAGAACCCGTGTTGGTGCA
AAGCTATGGGTGGAACATAGTCCGTCTTAAGAGTG
GGGTGGACGTCTTTCATATGGCGGCTGAGCCGTGT
GACACGTTGCTGTGTGACATAGGTGAGTCATCATC
TAGTCCTGAAGTGGAAGAAGCACGGACGCTCAGAG
TCCTCTCCATGGTGGGGGATTGGCTTGAAAAAAGA
CCAGGAGCCTTTTGTATAAAAGTGTTGTGCCCATA
CACCAGCACTATGATGGAAACCCTGGAGCGACTGC
AGCGTAGGTATGGGGGAGGACTGGTCAGAGTGCCA
CTCTCCCGCAACTCTACACATGAGATGTACTGGGT
CTCTGGAGCGAAAAGCAACACCATAAAAAGTGTGT
CCACCACGAGCCAGCTCCTCTTGGGGCGCATGGAC
GGGCCTAGGAGGCCAGTGAAATATGAGGAGGATGT
GAATCTCGGCTCTGGCACGCGGGCTGTGGTAAGCT
GCGCTGAAGCTCCCAACATGAAGATCATTGGTAAC
CGCATTGAAAGGATCCGCAGTGAGCACGCGGAAAC
GTGGTTCTTTGACGAGAACCACCCATATAGGACAT
GGGCTTACCATGGAAGCTATGAGGCCCCCACACAA
GGGTCAGCGTCCTCTCTAATAAACGGGGTTGTCAG
GCTCCTGTCAAAACCCTGGGATGTGGTGACTGGAG
TCACAGGAATAGCCATGACCGACACCACACCGTAT
GGTCAGCAAAGAGTTTTCAAGGAAAAAGTGGACAC
TAGGGTGCCAGACCCCCAAGAAGGCACTCGTCAGG
TTATGAGCATGGTCTCTTCCTGGTTGTGGAAAGAG
CTAGGCAAACACAAACGGCCACGAGTCTGTACCAA
AGAAGAGTTCATCAACAAGGTTCGTAGCAATGCAG
CATTAGGGGCAATATTTGAAGAGGAAAAAGAGTGG
AAGACTGCAGTGGAAGCTGTGAACGATCCAAGGTT
CTGGGCTCTAGTGGACAAGGAAAGAGAGCACCACC
TGAGAGGAGAGTGCCAGAGTTGTGTGTACAACATG
ATGGGAAAAAGAGAAAAGAAACAAGGGGAATTTGG
AAAGGCCAAGGGCAGCCGCGCCATCTGGTATATGT
GGCTAGGGGCTAGATTTCTAGAGTTCGAAGCCCTT
GGATTCTTGAACGAGGATCACTGGATGGGGAGAGA
GAACTCAGGAGGTGGTGTTGAAGGGCTGGGATTAC
AAAGACTCGGATATGTCCTAGAAGAGATGAGTCGC
ATACCAGGAGGAAGGATGTATGCAGATGACACTGC
TGGCTGGGACACCCGCATCAGCAGGTTTGATCTGG
AGAATGAAGCTCTAATCACCAACCAAATGGAGAAA
GGGCACAGGGCCTTGGCATTGGCCATAATCAAGTA
CACATACCAAAACAAAGTGGTAAAGGTCCTTAGAC
CAGCTGAAAAAGGGAAAACAGTTATGGACATTATT
TCGAGACAAGACCAAAGGGGGAGCGGACAAGTTGT
CACTTACGCTCTTAACACATTTACCAACCTAGTGG
TGCAACTCATTCGGAATATGGAGGCTGAGGAAGTT
CTAGAGATGCCTAGAGATGCAAGACTTGTGGCTGC
TGCGGAGGTCAGAGAAAGTGACCAACTGGTTGCAG
AGCAACGGATGGGATAGGCTCAAACGAATGGCAGT
CAGTGGAGATGATTGCGTTGTGAAGCCAATTGATG
ATAGGTTTGCACATGCCCTCAGGTTCTTGAATGAT
ATGGGAAAAGTTAGGAAGGACACACAAGAGTGGAA
ACCCTCAACTGGATGGGACAACTGGGAAGAAGTTC
CGTTTTGCTCCCACCACTTCAACAAGCTCCATCTC
AAGGACGGGAGGTCCATTGTGGTTCCCTGCCGCCA
CCAAGATGAACTGATTGGCCGGGCCCGCGTCTCTC
CAGGGGCGGGATGGAGCATCCGGGAGACTGCTTGC
CTAGCAAAATCATATGCGCAAATGTGGCAGCTCCT
TTATTTCCACAGAAGGGACCTCCGACTGATGGCCA
ATGCCATTTGTTCATCTGTGCCAGTTGACTGGGTT
CCAACTGGGAGAACTACCTGGTCAATCCATGGAAA
GGGAGAATGGATGACCACTGAAGACATGCTTGTGG
TGTGGAACAGAGTGTGGATTGAGGAGAACGACCAC
ATGGAAGACAAGACCCCAGTTACGAAATGGACAGA
CATTCCCTATTTGGGAAAAAGGGAAGACTTGTGGT
GTGGATCTCTCATAGGGCACAGACCGCGCACCACC
TGGGCTGAGAACATTAAAAACACAGTCAACATGGT
GCGCAGGATCATAGGTGATGAAGAAAAGTACATGG
ACTACCTATCCACCCAAGTTCGCTACTTGGGTGAA
GAAGGGTCTACACCTGGAGTGCTG.

As would be understood by the person skilled in the art, the target of the methods of the present disclosure is an RNA. In some examples, the target is a viral RNA. Thus, in some examples, the method may comprise the steps of purifying RNA from the sample. In some examples, the method may comprise the steps of cDNA synthesis. In some examples, the purified (viral) RNA is prepared into cDNA.

In some examples, the detecting and/or differentiating and/or quantifying comprises performing reverse transcription polymerase chain reaction (RT-PCR). In some examples, it would be well understood that the consensus sequences as described herein may be translated into amino acid sequences that could be used to generate peptides for use in serological assays.

In some examples, the detection and/or differentiation and/or quantification of the virus is performed by subjecting the sample to a reverse transcription polymerase chain reaction (RT-PCR) using primers and probe specific to the target regions or fragments thereof. In some examples, the sequences as described herein (such as the consensus sequences of each of the viruses) were sequences of clinically important isolates/strains worldwide that are retrieved from the art.

As used herein, the term “primer” refers to an oligonucleotide that acts as a point of initiation of DNA (or cDNA) synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. In some examples, a primer may be a single-stranded oligodeoxyribonucleotide. The primer may include a “hybridizing region” exactly or substantially complementary to the target sequence, for example about 15 to about 35 nucleotides in length, or 20, or 21, or 22, or 23, or 24, or 25, or 26, or 27, or 28, or 29, or 30, or 31, or 32, or 33, or 34, or 35 nucleotides in length. A primer oligonucleotide may either consist entirely of the hybridizing region or may contain additional features which allow for the detection, differentiation, quantification, immobilization, or manipulation of the amplified product, but which do not alter the ability of the primer to serve as a starting reagent for DNA (or cDNA) synthesis. For example, a nucleic acid sequence tail can be included at the 5′ end of the primer that hybridizes to a capture oligonucleotide.

As used herein, the term “probe” refers to an oligonucleotide that selectively hybridizes to a target nucleic acid under suitable conditions. A probe for detection of the target region as described herein may be of any length for example about 15 to 35 nucleotides length, or 20, or 21, or 22, or 23, or 24, or 25, or 26, or 27, or 28, or 29, or 30, or 31, or 32, or 33, or 34, or 35 nucleotides in length.

In some examples, the primers and probe may include, but is not limited to the following exemplary primers and probe:

a ZIKV forward primer comprising a sequence at least 90% identical to CCTTGGATTCTTGAACGAGGATCAC (NS5 ZIKV-F/SEQ ID NO: 7);

a ZIKV reverse primer comprising a sequence at least 90% identical to GCTTCATTCTCCAGATCAAACCTGC (NS5_ZIKV-R/SEQ ID NO: 9) or GCTTCATTCTCTAGATCAAACCTGC (ZIKV-R1_T/SEQ ID NO: 32);

a ZIKV probe comprising a sequence at least 90% identical to TACCAGGAGGAAGGATGTATGCAG (5′-FAM NS5_ZIKV-P/ZEN/3′ IBFQ/SEQ ID NO: 8) or ACCAGGAGGAAAGATGTACGCAG (ZIKV_P1_AF/SEQ ID NO: 33);

a DENV1 first forward primer comprising a sequence at least 90% identical to GGCTGAAGAAAGTCACAGAAG (NS5_D1-F_A/SEQID NO: 10);

a DENV1 second forward primer comprising a sequence at least 90% identical to GGCTGAAGAAAGTCACTGAAG (NS5_D1-F_T/SEQID NO: 11);

a DENV1 reverse primer comprising a sequence at least 90% identical to GAGGACTCACCAATATCACACAA (NS5_D1-R/SEQ ID NO: 13); a DENV1 probe comprising a sequence at least 90% identical to

ACCTATGGATGGAACCTAGTAAAGCT (5′-HEX NS5_D1/ZEN/3′ IBFQ/SEQ ID NO: 12);

a DENV3 forward primer comprising a sequence at least 90% identical to GCTCAGCCTCCTCCATGATAAATG (NS5_D3-F/SEQ ID NO: 14);

a DENV3 reverse primer comprising a sequence at least 90% identical to GGGTGTCCTGGTGTCCACTTTCTC (NS5_D3-R/SEQ ID NO: 17);

a DENV3 first probe comprising a sequence at least 90% identical to CATGGTGACACAGATGGCAATGAC (5′-Texas Red NS5_D3-P_T/3′ IBRQ/SEQ ID NO: 15);

a DENV3 second probe comprising a sequence at least 90% identical to CACGGTGACACAGATGGCAATGAC (5′-Texas Red NS5_D3-P_C/3′ IBRQ/SEQID NO: 16);

a CHIKV forward primer comprising a sequence at least 90% identical to GGCGCCTACTGCTTCTGCGAC (E1_CHIKV-F1/SEQID NO: 18);

a CHIKV reverse primer comprising a sequence at least 90% identical to TTGGTAAAGGACGCGGAGCTTAGC (E1_CHIKV-R1/SEQ ID NO: 21);

a CHIKV first probe comprising a sequence at least 90% identical to AGCGAAGCACATGTGGAGAAGTCC (5′-FAM E1_CHIKV-P1_T/ZEN/3′ IBFQ/SEQID NO:

19);

a CHIKV second probe comprising a sequence at least 90% identical to AGCGAAGCACACGTGGAGAAGTCC (5′-FAM E1_CHIKV-P1_C/ZEN/3′ IBFQ/SEQID NO: 20);

a DENV2 forward primer comprising a sequence at least 90% identical to ACACAGATGGCAATGACAGACACG (NS5_D2-F2/SEQ ID NO: 22);

a DENV2 reverse primer comprising a sequence at least 90% identical to CCAAGGCTGCATTGCTTCTCAC (NS5_D2-R2/SEQ ID NO: 24);

a DENV2 probe comprising a sequence at least 90% identical to TGGAAAGAACTAGGAAAGAAAAAGACAC (5′-HEX NS5_D2-P2/ZEN/3′ IBFQ/SEQ ID NO: 23);

a DENV4 first forward primer comprising a sequence at least 90% identical to TGGTTAGACCACCTTTCAATATG (C_D4-F1.2_T/SEQID NO: 25);

a DENV4 second forward primer comprising a sequence at least 90% identical to TGGCTAGACCACCTTTCAATATG (C_D4-F1.2_C/SEQID NO: 26);

a DENV4 reverse primer comprising a sequence at least 90% identical to TGCTAGCACCATCCGTAA (C_D4-R1.2/SEQ ID NO: 28);

a DENV4 probe comprising a sequence at least 90% identical to CCTCAAGGGTTGGTGAAGAGATTC (5′-Texas Red C_D4-P1/3′ IBRQ/SEQID NO: 27); and a combination thereof.

In some examples, the primers and/or probe may comprise a sequence at least 95% or at least 96% or at least 97% or at least 98% or at least 99% or are identical to SEQ ID NO: 7 to SEQ ID NO: 28. In some examples, the primers and/or probe may comprise a sequence having 10 or less nucleic acid difference, or 9 or less nucleic acid difference, or 8 or less nucleic acid difference, or 7 or less nucleic acid difference, or 6 or less nucleic acid difference, or 5 or less nucleic acid difference, or 4 or less nucleic acid difference, or 3 or less nucleic acid difference, or 2 or less nucleic acid difference or one or two nucleic acid difference from SEQ ID NO: 7 to SEQ ID NO: 28.

In some examples, the primers and/or probes may be conjugated with a detectable label. In some examples, the detectable label may provide signals detectable by fluorescence, radioactivity, colorimetric, X-ray diffraction or absorption, magnetism, enzymatic activity, and the like. In some examples, the detectable label may include but is not limited to a fluorophore, a radioactive agent, a colorimetric agent, a gravimetric agent, a detectable enzyme, a quencher, and their combination thereof. In some examples, the primer and/or probes may comprise one or more quencher, or two quenchers, or three quenchers, or more. For example, the fluorophore may include, but is not limited to, 5′-FAM (also called 5′-carboxyfluorescein; also called Spiro(isobenzofuran-1(3H), 9′-(9H)xanthene)-5-carboxylic acid,3′,6′-dihydroxy-3-oxo-6-carboxyfluorescein); 5′-HEX (also called 5-Hexachloro-Fluorescein([4,7,2′,4′,5′,7′-hexachloro-(3′,6′-dipivaloyl-fluoresceinyl)-6-carboxylic acid])); 6-Hexachloro-Fluorescein([4,7,2′,4′,5′,7′-hexachloro-(3′,6′-dipivaloylfluoresceinyI)-5-carboxylic acid]); 5,-Tetrachloro-Fluorescein ([4,7,2′,7′-tetra-chloro-(3′,6′-dipivaloylfluoresceinyl)-5-carboxylic acid]); 6-Tetrachloro-Fluorescein([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl- amino); 6-TAMRA (6-carboxytetramethylrhodamine; Xanthylium, 9-(2,5-dicarboxyphenyl)-3, 6-bis(dimethylamino); EDANS (5-((2-aminoethyl) amino)naphthalene-1-sulfonic acid); 1,5-IAEDANS (5-((((2-iodoacetyl)amino)ethyl) amino)naphthalene-1-sulfonic acid); DABCYL (4-((4-(dimethylamino)phenyl)azo)benzoic acid)Cy5, (Indodicarbocyanine-5)Cy3 (Indo- dicarbocyanine-3); and BODIPY FL (2,6-dibromo-4,4-difluoro-5,7-dimethyl-4-bora-3a,4a-diaza-s-indacene-3-proprionic acid), Quasar-670 (Biosearch Technologies), CalOrange (Biosearch Technologies), Rox, FAM, HEX, Cy5™, Texas Red®, and suitable derivatives thereof.

In some examples, the fluorophore may include FAM (carboxyfluorescein), HEX (Hexachlorofluorescein), Texas Red®, Cy5™ and the like.

As used herein, the term “quencher” refers to a chromophoric molecule or part of a compound, which is capable of reducing the emission from a fluorescent donor when attached to or in proximity to the donor. Quenching may occur by any of several mechanisms including fluorescence resonance energy transfer, photo-induced electron transfer, paramagnetic enhancement of intersystem crossing, Dexter exchange coupling, and exciton coupling such as the formation of dark complexes. Fluorescence is “quenched” when the fluorescence emitted by the fluorophore is reduced as compared with the fluorescence in the absence of the quencher by at least 10%, for example,15%, 20%, 30%, 40%, 50%, 60%, 70%, 80%, 90%, 95%, 98%, 99%, 99.9% or more.

The quencher can be any material that can quench at least one fluorescence emission from an excited fluorophore being used in the assay.

A number of commercially available quenchers are known in the art, and include but are not limited to ZEN™, TAO™, and the like, developed by Integrated DNA Technologies (IDT). In some examples, the quenchers ZEN ™ and/or TAOTM may be used in addition to 3′quencher Iowa Black FQ (IBFQ) or 3′ IBRQ quencher, resulting in double-quenched probes, for example, e.g. 5′-FAM/ZEN/3′IBFQ or 5′-CY5/TAO/3′IBRQ. The inventors found that these double quenched probes generate less background and have increased signal compared to probes containing single quencher. Each probe's fluorophore is selected with the least amount of spectral overlap.

The methods of the present disclosure have been found to be useful in determining the specific virus that is causing various symptoms in a subject. Thus, in some embodiments, the sample is obtained from a subject suspected to have one (or more) of CHIKV, DENV1, DENV2, DENV3, DENV4, or ZIKV.

As shown in the Experimental section below, the methods of the present disclosure may be used on various samples. As used herein, the term “sample” may refer to a specimen that may contain the target of interest (i.e. virus of interest), which includes the nucleic acid sequences in or derived from the target of interest. Samples may be from any source such as biological specimens or environmental sources. Biological specimens include any tissue or material derived from a living or dead organism that may contain a target of interest or nucleic acid in or derived from the target of interest. Examples of biological samples include respiratory tissue, exudates (e.g., bronchoalveolar lavage), biopsy, sputum, whole blood (such as peripheral blood), plasma, serum, lymph node, gastrointestinal tissue, feces, urine, or other fluids, tissues or materials. Examples of environmental samples include water, ice, soil, slurries, debris, biofilms, airborne particles, and aerosols. Samples may be processed specimens or materials, such as obtained from treating a sample by using filtration, centrifugation, sedimentation, or adherence to a medium, such as matrix or support. Other processing of samples may include treatments to physically or mechanically disrupt tissue, cellular aggregates, or cells to release intracellular components that include nucleic acids into a solution which may contain other components, such as enzymes, buffers, salts, detergents and the like. In some examples, the sample may include, but is not limited to, whole blood, serum, plasma, cerebrospinal fluid, urine, and amniotic fluid. In some example, the sample may be whole blood.

In some examples, the sample may be whole blood treated with Ethylenediaminetetraacetic acid (EDTA). In some examples, the sample may be whole blood treated with EDTA and at least one other biological sample obtained from the same patient (i.e. patient-matched whole blood specimen) including serum, cerebrospinal fluid (CSF), urine, amniotic fluid, and the like.

In the process of developing the methods of the present disclosure, the inventors of the present disclosure found that ZIKV RNA is generally detectable in serum, whole blood and/or urine during the acute phase of infection and up to 14 days following onset of symptoms. Thus, in some examples, the sample for detecting or differentiating Zika virus may be serum, whole blood, and/or urine.

In some examples, the sample may be obtained from various phase of infection. For example, for detection of Zika virus, the sample may be obtained during acute phase of infection. In some examples, the sample may be obtained up to 14 days following onset of symptoms (if present). For detection of CHIKV and/or either one of the four serotypes of DENV, the sample may be obtained during the acute phase of the disease. In some examples, the sample may be obtained less than 14 to 1, or 14, or 13, or 12, or 11, or 10, or 9, or 8, or 7, or 6, or 5, or 4, or 3, or 2 days post-illness onset. In some examples, the sample may be obtained less than 7 days post-illness onset.

As would be apparent to the person skilled in the art, a positive result would be indicative of a current infection. On the other hand, negative result (such as negative RT-PCR result) may not rule out infections by one or more of CHIKV, DENV1, DENV2, DENV3, and/or ZIKV infections and should not be used as the sole basis for patient management decisions. It would be apparent to the skilled artisan that a negative result may be combined with clinical observations, patient history, and epidemiological information. An exemplary decision algorithm for positive and negative results observed can be seen in Table 4 (see Experimental section).

In some examples, the methods as disclosed herein may further include the inclusion or addition of an internal control. As used herein, the term “internal control” refers to any substance or mixture of known composition that is added to or is part of the sample that is used to establish a baseline for comparison with the target of interest. For example, the internal control may be added to the sample or may be a region of a molecule known to be present in the sample. Under the simultaneous presence of the target region and the internal control, the target region and internal control are subjected to identical conditions within the method or assay, providing an explicit measure of the effectiveness of the entire method or assay or test system. In some examples, the internal control may be any endogenous target that are detactable in blood. In some examples, the internal control may include, but is not limited to, GAPDH, beta-globin, beta-actin, and the like.

In some example, the internal control may be detected by the oligonucleotide comprising or consisting of:

a beta-actin forward primer comprising a sequence at least 90% identical to GGCACCCAGCACAATGAAG (B-actin-F; SEQ ID NO: 29);

a beta-actin reverse primer comprising a sequence at least 90% identical to GCCGATCCACACGGAGTACT (B-actin-R; SEQ ID NO: 31);

a beta-actin probe comprising a sequence at least 90% identical to TCAAGATCATTGCTCCTCCTGAGAGCGC (5′-Cy5 B-actin-P/TAO/3′ IBRQ; SEQ ID NO: 30).

In some examples, the primers and/or probe may comprise a sequence at least 95% or at least 96% or at least 97% or at least 98% or at least 99% or are identical to SEQ ID NO: 29 to SEQ ID NO: 31. In some examples, the primers and/or probe may comprise a sequence having 10 or less nucleic acid difference, or 9 or less nucleic acid difference, or 8 or less nucleic acid difference, or 7 or less nucleic acid difference, or 6 or less nucleic acid difference, or 5 or less nucleic acid difference, or 4 or less nucleic acid difference, or 3 or less nucleic acid difference, or 2 or less nucleic acid difference or one or two nucleic acid difference from SEQ ID NO: 29 to SEQ ID NO: 31.

In another aspect, there is provided an isolated oligonucleotide for a simultaneous detection and/or differentiate and/or quantify virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, wherein the oligonucleotide detects a nucleic acid sequence that is at least 80% identical to the sequences selected from the group consisting of:

a nucleic acid molecule that encodes a nucleotide sequence of Non Structural protein 5 (NS5) of Zika virus,

a nucleic acid molecule that encodes a nucleotide sequence of NS5 of DENV1,

a nucleic acid molecule that encodes a nucleotide sequence of NS5 of DENV2,

a nucleic acid molecule that encodes a nucleotide sequence of NS5 of DENV3,

a nucleic acid molecule that encodes a nucleotide sequence of Capsid of DENV4, and

a nucleic acid molecule that encodes a nucleotide sequence of E1 glycoprotein of CHIKV.

In some examples, there is provided an isolated oligonucleotide for a simultaneous detection and/or differentiate and/or quantify three or more virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, wherein the oligonucleotide detects a nucleic acid sequence that is at least 80% identical to the sequences selected from the group consisting of:

a nucleic acid molecule that encodes a nucleotide sequence of Non Structural protein 5 (NS5) of Zika virus,

a nucleic acid molecule that encodes a nucleotide sequence of NS5 of DENV1,

a nucleic acid molecule that encodes a nucleotide sequence of NS5 of DENV2,

a nucleic acid molecule that encodes a nucleotide sequence of NS5 of DENV3,

a nucleic acid molecule that encodes a nucleotide sequence of Capsid of DENV4, and

a nucleic acid molecule that encodes a nucleotide sequence of E1 glycoprotein of CHIKV.

Thus, in some examples, the isolated oligonucleotide is capable of detecting three or more viruses, or four or more viruses, or five or more viruses, or all six viruses, which includes Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV).

In some examples, the nucleotide sequence of E1 glycoprotein of CHIKV comprises SEQ ID NO: 1 or its fragment or parts thereof, the nucleotide sequence of NS5 of DENV1 comprises SEQ ID NO: 2 or its fragment or parts thereof, the nucleotide sequence of NS5 of DENV2 comprises SEQ ID NO: 3 or its fragment or parts thereof, the nucleotide sequence of NS5 of DENV3 comprises SEQ ID NO: 4 or its fragment or parts thereof, the nucleotide sequence of Capsid of DENV4 comprises SEQ ID NO: 5 or its fragment or parts thereof, and the nucleotide sequence of Non Structural protein 5 (NS5) of Zika virus comprises SEQ ID NO: 6 or its fragment or parts thereof.

In some examples, the oligonucleotide may include, but is not limited to (or comprise or consist of):

a ZIKV forward primer comprising a sequence at least 90% identical to CCTTGGATTCTTGAACGAGGATCAC (SEQ ID NO: 7);

a ZIKV reverse primer comprising a sequence at least 90% identical to GCTTCATTCTCCAGATCAAACCTGC (SEQ ID NO: 9) or GCTTCATTCTCTAGATCAAACCTGC (SEQ ID NO: 32);

a ZIKV probe comprising a sequence at least 90% identical to TACCAGGAGGAAGGATGTATGCAG (SEQ ID NO: 8) or ACCAGGAGGAAAGATGTACGCAG (SEQ ID NO: 33);

a DENV1 first forward primer comprising a sequence at least 90% identical to GGCTGAAGAAAGTCACAGAAG (SEQ ID NO: 10);

a DENV1 second forward primer comprising a sequence at least 90% identical to GGCTGAAGAAAGTCACTGAAG (SEQ ID NO: 11);

a DENV1 reverse primer comprising a sequence at least 90% identical to GAGGACTCACCAATATCACACAA (SEQ ID NO: 13);

a DENV1 probe comprising a sequence at least 90% identical to ACCTATGGATGGAACCTAGTAAAGCT (SEQ ID NO: 12);

a DENV3 forward primer comprising a sequence at least 90% identical to GCTCAGCCTCCTCCATGATAAATG (SEQ ID NO: 14);

a DENV3 reverse primer comprising a sequence at least 90% identical to GGGTGTCCTGGTGTCCACTTTCTC (SEQ ID NO: 17);

a DENV3 first probe comprising a sequence at least 90% identical to CATGGTGACACAGATGGCAATGAC (SEQ ID NO: 15);

a DENV3 second probe comprising a sequence at least 90% identical to CACGGTGACACAGATGGCAATGAC (SEQ ID NO: 16);

a CHIKV forward primer comprising a sequence at least 90% identical to GGCGCCTACTGCTTCTGCGAC (SEQ ID NO: 18);

a CHIKV reverse primer comprising a sequence at least 90% identical to TTGGTAAAGGACGCGGAGCTTAGC (SEQ ID NO: 21);

a CHIKV first probe comprising a sequence at least 90% identical to AGCGAAGCACATGTGGAGAAGTCC (SEQ ID NO: 19);

a CHIKV second probe comprising a sequence at least 90% identical to AGCGAAGCACACGTGGAGAAGTCC (SEQ ID NO: 20);

a DENV2 forward primer comprising a sequence at least 90% identical to ACACAGATGGCAATGACAGACACG (SEQ ID NO: 22);

a DENV2 reverse primer comprising a sequence at least 90% identical to CCAAGGCTGCATTGCTTCTCAC (SEQ ID NO: 24);

a DENV2 probe comprising a sequence at least 90% identical to TGGAAAGAACTAGGAAAGAAAAAGACAC (SEQ ID NO: 23);

a DENV4 first forward primer comprising a sequence at least 90% identical to TGGTTAGACCACCTTTCAATATG (SEQ ID NO: 25);

a DENV4 second forward primer comprising a sequence at least 90% identical to TGGCTAGACCACCTTTCAATATG (SEQ ID NO: 26);

a DENV4 reverse primer comprising a sequence at least 90% identical to TGCTAGCACCATCCGTAA (SEQ ID NO: 28); and

a DENV4 probe comprising a sequence at least 90% identical to CCTCAAGGGTTGGTGAAGAGATTC (SEQ ID NO: 27).

In some examples, the oligonucleotides as disclosed herein may further include oligonucleotides for detecting an internal control. In some example, the oligonucleotide for detecting internal control may include, but is not limited to:

a beta-actin forward primer comprising a sequence at least 90% identical to GGCACCCAGCACAATGAAG (B-actin-F; SEQ ID NO: 29);

a beta-actin reverse primer comprising a sequence at least 90% identical to GCCGATCCACACGGAGTACT (B-actin-R; SEQ ID NO: 31);

a beta-actin probe comprising a sequence at least 90% identical to TCAAGATCATTGCTCCTCCTGAGAGCGC (5′-Cy5 B-actin-P/TAO/3′ IBRQ; SEQ ID NO: 30).

In some examples, the oligonucleotide is at least 95%, 96%, 97%, 98%, 99%, or 100% identical to SEQ ID NO: 7 to SEQ ID NO: 31. In some examples, the oligonucleotide may comprise a sequence at least 95% or at least 96% or at least 97% or at least 98% or at least 99% or are identical to SEQ ID NO: 7 to SEQ ID NO: 31. In some examples, the oligonucleotide may comprise a sequence having 10 or less nucleic acid difference, or 9 or less nucleic acid difference, or 8 or less nucleic acid difference, or 7 or less nucleic acid difference, or 6 or less nucleic acid difference, or 5 or less nucleic acid difference, or 4 or less nucleic acid difference, or 3 or less nucleic acid difference, or 2 or less nucleic acid difference or one or two nucleic acid difference from SEQ ID NO: 7 to SEQ ID NO: 31.

In another aspect, there is provided a method for detecting and/or differentiating and/or quantifying virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, the method comprising:

subjecting the sample to a reverse transcription polymerase chain reaction (RT-PCR) using primers and a probe specific for CHIKV E1 glycoprotein, primers and a probe specific for DENV1 Non Structural protein 5 (NS5), primers and a probe specific for DENV2 NS5, primers and a probe specific for DENV3 NS5, primers and a probe specific for DENV4 capsid, and primers and a probe specific for ZIKV NS5.

In some examples, there is provided a method for detecting and/or differentiating and/or quantifying three or more virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, the method comprising:

subjecting the sample to a reverse transcription polymerase chain reaction (RT-PCR) using primers and a probe specific for CHIKV E1 glycoprotein, primers and a probe specific for DENV1 Non Structural protein 5 (NS5), primers and a probe specific for DENV2 NS5, primers and a probe specific for DENV3 NS5, primers and a probe specific for DENV4 capsid, and primers and a probe specific for ZIKV NS5. In some examples, the method is capable of detecting three or more viruses, or four or more viruses, or five or more viruses, or all six viruses, which includes Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV).

In some example, the primers and probes may comprise:

a ZIKV forward primer comprising a sequence at least 90% identical to CCTTGGATTCTTGAACGAGGATCAC (SEQ ID NO: 7);

a ZIKV reverse primer comprising a sequence at least 90% identical to GCTTCATTCTCCAGATCAAACCTGC (SEQ ID NO: 9) or GCTTCATTCTCTAGATCAAACCTGC (SEQ ID NO: 32);

a ZIKV probe comprising a sequence at least 90% identical to TACCAGGAGGAAGGATGTATGCAG (SEQ ID NO: 8) or ACCAGGAGGAAAGATGTACGCAG (SEQ ID NO: 33);

a DENV1 first forward primer comprising a sequence at least 90% identical to GGCTGAAGAAAGTCACAGAAG (SEQ ID NO: 10);

a DENV1 second forward primer comprising a sequence at least 90% identical to GGCTGAAGAAAGTCACTGAAG (SEQ ID NO: 11);

a DENV1 reverse primer comprising a sequence at least 90% identical to GAGGACTCACCAATATCACACAA (SEQ ID NO: 13);

a DENV1 probe comprising a sequence at least 90% identical to ACCTATGGATGGAACCTAGTAAAGCT (SEQ ID NO: 12);

a DENV3 forward primer comprising a sequence at least 90% identical to GCTCAGCCTCCTCCATGATAAATG (SEQ ID NO: 14);

a DENV3 reverse primer comprising a sequence at least 90% identical to GGGTGTCCTGGTGTCCACTTTCTC (SEQ ID NO: 17);

a DENV3 first probe comprising a sequence at least 90% identical to CATGGTGACACAGATGGCAATGAC (SEQ ID NO: 15);

a DENV3 second probe comprising a sequence at least 90% identical to CACGGTGACACAGATGGCAATGAC (SEQ ID NO: 16);

a CHIKV forward primer comprising a sequence at least 90% identical to GGCGCCTACTGCTTCTGCGAC (SEQ ID NO: 18);

a CHIKV reverse primer comprising a sequence at least 90% identical to TTGGTAAAGGACGCGGAGCTTAGC (SEQ ID NO: 21);

a CHIKV first probe comprising a sequence at least 90% identical to AGCGAAGCACATGTGGAGAAGTCC (SEQ ID NO: 19);

a CHIKV second probe comprising a sequence at least 90% identical to AGCGAAGCACACGTGGAGAAGTCC (SEQ ID NO: 20);

a DENV2 forward primer comprising a sequence at least 90% identical to ACACAGATGGCAATGACAGACACG (SEQ ID NO: 22);

a DENV2 reverse primer comprising a sequence at least 90% identical to CCAAGGCTGCATTGCTTCTCAC (SEQ ID NO: 24);

a DENV2 probe comprising a sequence at least 90% identical to TGGAAAGAACTAGGAAAGAAAAAGACAC (SEQ ID NO: 23);

a DENV4 first forward primer comprising a sequence at least 90% identical to TGGTTAGACCACCTTTCAATATG (SEQ ID NO: 25);

a DENV4 second forward primer comprising a sequence at least 90% identical to TGGCTAGACCACCTTTCAATATG (SEQ ID NO: 26);

a DENV4 reverse primer comprising a sequence at least 90% identical to TGCTAGCACCATCCGTAA (SEQ ID NO: 28); and

a DENV4 probe comprising a sequence at least 90% identical to CCTCAAGGGTTGGTGAAGAGATTC (SEQ ID NO: 27).

In some examples, the primers and/or probe may comprise a sequence at least 95% or at least 96% or at least 97% or at least 98% or at least 99% or are identical to SEQ ID NO: 7 to SEQ ID NO: 28. In some examples, the primers and/or probe may comprise a sequence having 10 or less nucleic acid difference, or 9 or less nucleic acid difference, or 8 or less nucleic acid difference, or 7 or less nucleic acid difference, or 6 or less nucleic acid difference, or 5 or less nucleic acid difference, or 4 or less nucleic acid difference, or 3 or less nucleic acid difference, or 2 or less nucleic acid difference or one or two nucleic acid difference from SEQ ID NO: 7 to SEQ ID NO: 28.

In some example, the oligonucleotide as disclosed herein and/or the primers and/or probe may be:

the ZIKV forward primer comprising
(SEQ ID NO: 7)
CCTTGGATTCTTGAACGAGGATCAC;
the ZIKV reverse primer comprising
(SEQ ID NO: 9)
GCTTCATTCTCCAGATCAAACCTGC
or
(SEQ ID NO: 32)
GCTTCATTCTCTAGATCAAACCTGC;
the ZIKV probe comprising
(SEQ ID NO: 8)
TACCAGGAGGAAGGATGTATGCAG
or
(SEQ ID NO: 33)
ACCAGGAGGAAAGATGTACGCAG;
the DENV1 first forward primer comprising
(SEQ ID NO: 10)
GGCTGAAGAAAGTCACAGAAG;
the DENV1 second forward primer comprising
(SEQ ID NO: 11)
GGCTGAAGAAAGTCACTGAAG;
the DENV1 reverse primer comprising
(SEQ ID NO: 13)
GAGGACTCACCAATATCACACAA;
the DENV1 probe comprising
(SEQ ID NO: 12)
ACCTATGGATGGAACCTAGTAAAGCT;
the DENV3 forward primer comprising
(SEQ ID NO: 14)
GCTCAGCCTCCTCCATGATAAATG;
the DENV3 reverse primer comprising
(SEQ ID NO: 17)
GGGTGTCCTGGTGTCCACTTTCTC;
the DENV3 first probe comprising
(SEQ ID NO: 15)
CATGGTGACACAGATGGCAATGAC;
the DENV3 second probe comprising
(SEQ ID NO: 16)
CACGGTGACACAGATGGCAATGAC;
the CHIKV forward primer comprising
(SEQ ID NO: 18)
GGCGCCTACTGCTTCTGCGAC;
the CHIKV reverse primer comprising
(SEQ ID NO: 21)
TTGGTAAAGGACGCGGAGCTTAGC;
the CHIKV first probe comprising
(SEQ ID NO: 19)
AGCGAAGCACATGTGGAGAAGTCC;
the CHIKV second probe comprising
(SEQ ID NO: 20)
AGCGAAGCACACGTGGAGAAGTCC;
the DENV2 forward primer comprising
(SEQ ID NO: 22)
ACACAGATGGCAATGACAGACACG;
the DENV2 reverse primer comprising
(SEQ ID NO: 24)
CCAAGGCTGCATTGCTTCTCAC;
the DENV2 probe comprising
(SEQ ID NO: 23)
TGGAAAGAACTAGGAAAGAAAAAGACAC;
the DENV4 first forward primer comprising
(SEQ ID NO: 25)
TGGTTAGACCACCTTTCAATATG;
the DENV4 second forward primer comprising
(SEQ ID NO: 26)
TGGCTAGACCACCTTTCAATATG;
the DENV4 reverse primer comprising
(SEQ ID NO: 28)
TGCTAGCACCATCCGTAA;
and
the DENV4 probe comprising
(SEQ ID NO: 27)
CCTCAAGGGTTGGTGAAGAGATTC.

In some examples, the primers and/or probe may comprise a sequence at least 95% or at least 96% or at least 97% or at least 98% or at least 99% or are identical to SEQ ID NO: 7 to SEQ ID NO: 28. In some examples, the primers and/or probe may comprise a sequence having 10 or less nucleic acid difference, or 9 or less nucleic acid difference, or 8 or less nucleic acid difference, or 7 or less nucleic acid difference, or 6 or less nucleic acid difference, or 5 or less nucleic acid difference, or 4 or less nucleic acid difference, or 3 or less nucleic acid difference, or 2 or less nucleic acid difference or one or two nucleic acid difference from SEQ ID NO: 7 to SEQ ID NO: 28.

In some examples, the primers and/or probes may be conjugated with a detectable label. In some examples, the detectable label may provide signals detectable by fluorescence, radioactivity, colorimetric, X-ray diffraction or absorption, magnetism, enzymatic activity, and the like. In some examples, the detectable label may include but is not limited to a fluorophore, a radioactive agent, a colorimetric agent, a gravimetric agent, a detectable enzyme, a quencher, and their combination thereof. In some examples, the primer and/or probes may comprise one or more quencher, or two quenchers, or three quenchers, or more. For example, the fluorophore may include, but is not limited to, 5′-FAM (also called 5′-carboxyfluorescein; also called Spiro(isobenzofuran-1(3H), 9′-(9H)xanthene)-5-carboxylic acid,3′,6′-dihydroxy-3-oxo-6-carboxyfluorescein); 5′-HEX (also called 5-Hexachloro-Fluorescein([4,7,2′,4′,5′,7′-hexachloro-(3′,6′-dipivaloyl-fluoresceinyl)-6-carboxylic acid])); 6-Hexachloro-Fluorescein ([4,7,2′,4′,5′,7′-hexachloro-(3′,6′-dipivaloylfluoresceinyI)-5-carboxylic acid]); 5,-Tetrachloro-Fluorescein ([4,7,2′,7′-tetra-chloro-(3′,6′-dipivaloylfluoresceinyl)-5-carboxylic acid]); 6-Tetrachloro-Fluorescein([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl- amino); 6-TAMRA (6-carboxytetramethylrhodamine; Xanthylium, 9-(2,5-dicarboxyphenyl)-3, 6-bis(dimethylamino); EDANS (5-((2-aminoethyl) amino)naphthalene-1-sulfonic acid); 1,5-IAEDANS (5-((((2-iodoacetyl)amino)ethyl) amino)naphthalene-1- sulfonic acid); DABCYL (4-((4-(dimethylamino)phenyl)azo)benzoic acid)Cy5, (Indodicarbocyanine-5)Cy3 (Indo- dicarbocyanine-3); and BODIPY FL (2,6-dibromo-4,4-difluoro-5,7-dimethyl-4-bora-3a,4a-diaza-s-indacene-3-proprionic acid), Quasar-670 (Biosearch Technologies), CalOrange (Biosearch Technologies), Rox, and suitable derivatives thereof. In some examples, the fluorophore may include FAM (carboxyfluorescein), HEX (Hexachlorofluorescein), Texas Red®, Cy5™ and the like.

In some examples, the methods as disclosed herein may further include the inclusion or addition of an internal control.

In some example, the internal control may be detected by the oligonucleotide including, but is not limited to:

a beta-actin forward primer comprising a sequence at least 90% identical to GGCACCCAGCACAATGAAG (B-actin-F; SEQ ID NO: 29);

a beta-actin reverse primer comprising a sequence at least 90% identical to GCCGATCCACACGGAGTACT (B-actin-R; SEQ ID NO: 31);

a beta-actin probe comprising a sequence at least 90% identical to TCAAGATCATTGCTCCTCCTGAGAGCGC (5′-Cy5 B-actin-P/TAO/3′ IBRQ; SEQ ID NO: 30).

In some examples, the primers and/or probe may comprise a sequence at least 95% or at least 96% or at least 97% or at least 98% or at least 99% or are identical to SEQ ID NO: 29 to SEQ ID NO: 31. In some examples, the primers and/or probe may comprise a sequence having 10 or less nucleic acid difference, or 9 or less nucleic acid difference, or 8 or less nucleic acid difference, or 7 or less nucleic acid difference, or 6 or less nucleic acid difference, or 5 or less nucleic acid difference, or 4 or less nucleic acid difference, or 3 or less nucleic acid difference, or 2 or less nucleic acid difference or one or two nucleic acid difference from SEQ ID NO: 29 to SEQ ID NO: 31.

In another aspect, there is provided a kit for detecting Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, comprising: an agent specific for detecting CHIKV E1 glycoprotein, an agent specific for detecting DENV1 Non Structural protein 5 (NS5), an agent specific for detecting DENV2 NS5, an agent specific for detecting DENV3 NS5, an agent specific for detecting DENV4 capsid, and an agent specific for detecting ZIKV NS5.

In some examples, the kit comprises an agent for detecting a region or fragment thereof having 80% sequence identity to SEQ ID NO: 1; an agent for detecting a region or fragment thereof having 80% sequence identity to SEQ ID NO: 2; an agent for detecting a region or fragment thereof having 80% sequence identity to SEQ ID NO: 3; an agent for detecting a region or fragment thereof having 80% sequence identity to SEQ ID NO: 4; an agent for detecting a region or fragment thereof having 80% sequence identity to SEQ ID NO: 5; and an agent for detecting a region or fragment thereof having 80% sequence identity to SEQ ID NO: 6.

In some examples, the agent may comprise primers and probes comprising:

a ZIKV forward primer comprising a sequence at least 90% identical to CCTTGGATTCTTGAACGAGGATCAC (SEQ ID NO: 7);

a ZIKV reverse primer comprising a sequence at least 90% identical to GCTTCATTCTCCAGATCAAACCTGC (SEQ ID NO: 9) or GCTTCATTCTCTAGATCAAACCTGC (SEQ ID NO: 32);

a ZIKV probe comprising a sequence at least 90% identical to TACCAGGAGGAAGGATGTATGCAG (SEQ ID NO: 8) or ACCAGGAGGAAAGATGTACGCAG (SEQ ID NO: 33);

a DENV1 first forward primer comprising a sequence at least 90% identical to GGCTGAAGAAAGTCACAGAAG (SEQ ID NO: 10);

a DENV1 second forward primer comprising a sequence at least 90% identical to GGCTGAAGAAAGTCACTGAAG (SEQ ID NO: 11);

a DENV1 reverse primer comprising a sequence at least 90% identical to GAGGACTCACCAATATCACACAA (SEQ ID NO: 13);

a DENV1 probe comprising a sequence at least 90% identical to ACCTATGGATGGAACCTAGTAAAGCT (SEQ ID NO: 12);

a DENV3 forward primer comprising a sequence at least 90% identical to GCTCAGCCTCCTCCATGATAAATG (SEQ ID NO: 14);

a DENV3 reverse primer comprising a sequence at least 90% identical to GGGTGTCCTGGTGTCCACTTTCTC (SEQ ID NO: 17);

a DENV3 first probe comprising a sequence at least 90% identical to CATGGTGACACAGATGGCAATGAC (SEQ ID NO: 15);

a DENV3 second probe comprising a sequence at least 90% identical to CACGGTGACACAGATGGCAATGAC (SEQ ID NO: 16);

a CHIKV forward primer comprising a sequence at least 90% identical to GGCGCCTACTGCTTCTGCGAC (SEQ ID NO: 18);

a CHIKV reverse primer comprising a sequence at least 90% identical to TTGGTAAAGGACGCGGAGCTTAGC (SEQ ID NO: 21);

a CHIKV first probe comprising a sequence at least 90% identical to AGCGAAGCACATGTGGAGAAGTCC (SEQ ID NO: 19);

a CHIKV second probe comprising a sequence at least 90% identical to AGCGAAGCACACGTGGAGAAGTCC (SEQ ID NO: 20);

a DENV2 forward primer comprising a sequence at least 90% identical to ACACAGATGGCAATGACAGACACG (SEQ ID NO: 22);

a DENV2 reverse primer comprising a sequence at least 90% identical to CCAAGGCTGCATTGCTTCTCAC (SEQ ID NO: 24);

a DENV2 probe comprising a sequence at least 90% identical to TGGAAAGAACTAGGAAAGAAAAAGACAC (SEQ ID NO: 23);

a DENV4 first forward primer comprising a sequence at least 90% identical to TGGTTAGACCACCTTTCAATATG (SEQ ID NO: 25);

a DENV4 second forward primer comprising a sequence at least 90% identical to TGGCTAGACCACCTTTCAATATG (SEQ ID NO: 26);

a DENV4 reverse primer comprising a sequence at least 90% identical to TGCTAGCACCATCCGTAA (SEQ ID NO: 28); and

a DENV4 probe comprising a sequence at least 90% identical to CCTCAAGGGTTGGTGAAGAGATTC (SEQ ID NO: 27).

In some examples, the primers and/or probe may comprise a sequence at least 95% or at least 96% or at least 97% or at least 98% or at least 99% or are identical to SEQ ID NO:

7 to SEQ ID NO: 28. In some examples, the primers and/or probe may comprise a sequence having 10 or less nucleic acid difference, or 9 or less nucleic acid difference, or 8 or less nucleic acid difference, or 7 or less nucleic acid difference, or 6 or less nucleic acid difference, or 5 or less nucleic acid difference, or 4 or less nucleic acid difference, or 3 or less nucleic acid difference, or 2 or less nucleic acid difference or one or two nucleic acid difference from SEQ ID NO: 7 to SEQ ID NO: 28.

In some examples, the methods or kits as disclosed herein may be provided such that the reagents, primers and/or probes, or oligonucleotides are provided in two or more set. For example, as exemplified in the Experimental section, the method as disclosed herein may be performed as two-tube reactions (that could be run concurrently in the same RT-PCR run) where a first tube determines the presence of ZIKV, DENV1, DENV3 and a second tube determines the presence of CH IKV, DENV2 and DENV4. It would be understood that other permutations of the two-tube reactions would also be within the scope of the present disclosure.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For an example, when “comprising” is used, reference to a “one” feature is also intended to be a reference to “at least one” of that feature. Terms such as “consisting”, “consist”, and the like, may, in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

The example embodiments may also be practiced with other computer system configurations, including handheld devices, multiprocessor systems/servers, microprocessor-based or programmable consumer electronics, network PCs, minicomputers, mainframe computers, personal digital assistants, mobile telephones and the like. Furthermore, the example embodiments may also be practiced in distributed computing environments where tasks are performed by remote processing devices that are linked through a wireless or wired communications network. In a distributed computing environment, program modules may be located in both local and remote memory storage devices.

It will be appreciated by a person skilled in the art that other variations and/or modifications may be made to the specific embodiments without departing from the scope of the invention as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

### Experimental Section

### Assay Design

Six sets of primers and probes to target unique regions of the 6 viruses were designed. The 6 primers and probe sets are constituted into 2 multiplex mixes, each with the ability to detect 3 viruses with the inclusion of an internal control (IC). The assay consists of a 2-tube reaction with specific oligonucleotide primers and dual labeled 5′-fluorescent (Taqman) probes for in-vitro, multiplex detection of ZIKV, DENV1, DENV3 and IC or/and CHIKV, DENV2, DENV4 and IC respectively. The IC used in the assay targets β-actin, a constitutively present protein. Multiplexing is facilitated by the targeting of each virus/IC with a different probe, i.e. each Taqman probe targets a single virus/IC and is conjugated to a fluorophore that emits fluorescence at different excitation wavelengths. The following is information on each of the targets in the multiplex assay:

### 1. Chikungunya Virus (CHIKV);

Family: Togaviridae

Genus: Alphavirus

Target region: E1 glycoprotein

ssRNA-positive strand, causal agent of Chikungunya Fever

### 2-5. Dengue Virus Serotype 1 to 4 (DENV1-4);

Family: Flaviviridae

Genus: Flavivirus

Target region: Non Structural protein 5 (NS5) (DENV1-3), Capsid (C) (DENV4)

ssRNA-positive strand, causal agent of Dengue Fever

### 6. Zika Virus (ZIKV); and

Family: Flaviviridae

Genus: Flavivirus

Target region: Non Structural protein 5 (NS5)

ssRNA-positive strand, causal agent of Zika Fever

### 7. Internal Control (IC).

Target region: β-actin

Internal control target region adapted from Mocellin et al., IL-10 stimulatory effects on human NK cells explored by gene profile analysis, Genes & Immunity 5, 621-630 (2004)

### Specimens Used

### For CHIKV, DENV, and ZIKV Testing:

Whole blood (treated with Ethylenediaminetetraacetic acid/EDTA);
Serum (collected in a serum separator tube, tube centrifuged prior to shipping to avoid hemolysis (where applicable)); and
Cerebrospinal fluid.

### For ZIKV Testing:

Urine; and
Amniotic fluid.

### Materials and Methods

### Reagents

For purification of viral RNA from plasma, serum or whole blood samples:
Qiagen QlAamp Viral RNA Kit (50 or 250) (Cat. No. 52904 or 52906) or equivalent;

### For RT-PCR Reaction:

ThermoFisher SuperScript® Ill Platinum 6 One-Step Quantitative RT-PCR System (Cat. No. 11732088) or equivalent

### Multiplex Assay Workflow

The six primers and probe sets are constituted into two multiplex mixes, each with the ability to detect three targets with the inclusion of an internal control (IC). The assay consists of a two-tube reaction with specific oligonucleotide primers and dual labelled 5′-fluorescent (Taqman®) probes for in vitro, multiplex detection of ZIKV, DENV1, DENV3 and IC or/and CHIKV, DENV2, DENV4 and IC, respectively. The internal control (IC) used in the assay targets β-actin, a constitutively present protein.

Multiplexing is facilitated by the targeting of each virus/IC with a different colored probe, i.e. each Taqman® probe targets a single virus/IC and is conjugated to a fluorophore that emits fluorescence at different excitation wavelengths. Integrated DNA Technology (IDT) has developed internal Zen™ and TAO™ quenchers. They are used in addition to the 3′quencher Iowa Black FQ (IBFQ) or 3′ IBRQ quencher, resulting in double-quenched probes, e.g. 5′-FAM/ZEN/3′IBFQ or 5′-Cy5/TAO/3′IBRQ, in the assay. These double-quenched probes generate less background and have an increased signal compared to probes containing a single quencher. Each probe's fluorophore is selected with the least amount of spectral overlap.

The 2-tube reaction consist of the following reagents:

### Tube 1:

1. ZIKV primers+5′-FAM ZIKV/ZEN/3′IBFQ

2. DENV1 primers+5′-HEX DENV1/ZEN/3′IBFQ

3. DENV3 primers+5′Texas Red DENV3/3′ IBRQ

4. IC primers+5′-Cy5 IC/TAO/3′IBRQ

### Tube 2:

1. CHIKV primers+5′-FAM CHIKV/ZEN/3′IBFQ

2. DENV2 primers+5′-HEX DENV2/ZEN/3′IBFQ

3. DENV4 primers+5′-Texas Red DENV4/3′ IBRQ

4. IC primers+5′-Cy5 IC/TAO/3′IBRQ

| | | Probe |
|---|---|---|
| Mix 1 | Mix 2 | fluorophore |
| ZIKV | CHIKV | FAM |
| DENV1 | DENV2 | Hex |
| DENV3 | DENV4 | TxR |
| IC | IC | Cy5 |

The 2-tube reaction consists of the following primers and probes:

| | SIgN Mix 1 Reaction | |
|---|---|---|
| No. | Components. | Name. |
| 1 | ZIKV Primers (F + R) | Mix 1 F + R* |
| | DENV-1 Primers | |
| | (F + R) | |
| | DENV-3 Primers | |
| | (F + R) | |
| 2 | IC Primers (F + R) | IC F + R |
| 3 | ZIKV Probe | ZIKV FAM |
| 4 | DENV-1 Probe | DENV-1 Hex |
| 5 | DENV-3 Probe | DENV-3 TxR |
| 6 | IC Probe | IC Cy5 |

*F: forward primer; R: reverse primer

| Mix 1 F + R* | Primer Stock | Concentration | |
|---|---|---|---|
| Components | (μM) | in Mix (μM) | 100 μL mix |
| ZIKV-F | 100 | 10 | 10.0 |
| ZIKV-P | 100 | 10 | 10.0 |
| DENV1-F_A | 100 | 10 | 10.0 |
| DENV1-F_T | 100 | 10 | 10.0 |
| DENV1-R | 100 | 10 | 10.0 |
| DENV3-F | 100 | 4 | 4.00 |
| DENV3-R | 100 | 4 | 4.00 |
| Water | — | — | 42.0 |

| No. | SIgN Mix 2 Reaction Components. | Name. |
|---|---|---|
| 1 | CHIKV Primers (F + R) | Mix 2 F + R^{#} |
| | DENV-2 Primers (F + R) | |
| | DENV-4 Primers (F + R) | |
| 2 | IC Primers (F + R) | IC F + R |
| 3 | CHIKV Probe | CHIKV FAM |
| 4 | DENV-2 Probe | DENV-2 Hex |
| 5 | DENV-4 Probe | DENV-4 TxR |
| 6 | IC Probe | IC Cy5 |

| Mix 2 F + R^{#} | Primer Stock | Concentration | |
|---|---|---|---|
| Components | (μM) | in Mix (μM) | 100 μL mix |
| CHIKV-F | 100 | 10 | 10 |
| CHIKV-R | 100 | 10 | 10 |
| DENV2-F | 100 | 10 | 10 |
| DENV2-R | 100 | 10 | 10 |
| DENV4-F_T | 100 | 10 | 4 |
| DENV4-F_C | 100 | 4 | 4 |
| DENV4-R | 100 | 4 | 4 |
| Water | | | 48 |

Prepare the qRT-PCR reaction mix⁺ as follows:

### Mix 1 Reaction Set-Up

| Components | Volume (μL) |
|---|---|
| SSIII | 12.5 |
| mastermix | |
| Mix1 F + R | 1.25 |
| IC F + R | 0.50 |
| ZIKV Probe | 0.50 |
| DENV-1 Probe | 0.50 |
| DENV-3 Probe | 0.25 |
| IC Probe | 0.375 |
| MgSO4 | 0.50 |
| RT enzyme mix | 0.50 |
| RNA template | 5.00 |
| Nuclease free | 3.125 |
| H₂O | |

### Mix 2 Reaction Set-Up

| Components | Volume (μL) |
|---|---|
| SSIII | 12.5 |
| mastermix | |
| Mix 2 F + R | 1.25 |
| IC F + R | 0.50 |
| CHIKV Probe | 0.50 |
| DENV-2 Probe | 0.50 |
| DENV-4 Probe | 0.25 |
| IC Probe | 0.375 |
| MgSO4 | 0.50 |
| RT enzyme mix | 0.50 |
| RNA template | 5.00 |
| Nuclease free | 3.125 |
| H₂O | |

⁺Note:
1 μL of vircell RNA and 5 μL of elute from the extraction of HC (healthy donor whole blood) were used for LoD runs. Nuclease free H₂O was adjusted to 2.125 μL.

Sequences of primers and probes used are as follows:

the ZIKV forward primer comprising
(SEQ ID NO: 7)
CCTTGGATTCTTGAACGAGGATCAC;
the ZIKV reverse primer comprising
(SEQ ID NO: 9)
GCTTCATTCTCCAGATCAAACCTGC
or
(SEQ ID NO: 32)
GCTTCATTCTCTAGATCAAACCTGC;
the ZIKV probe comprising
(SEQ ID NO: 8)
TACCAGGAGGAAGGATGTATGCAG
or
(SEQ ID NO: 33)
ACCAGGAGGAAAGATGTACGCAG;
the DENV1 first forward primer comprising
(SEQ ID NO: 10)
GGCTGAAGAAAGTCACAGAAG;
the DENV1 second forward primer comprising
(SEQ ID NO: 11)
GGCTGAAGAAAGTCACTGAAG;
the DENV1 reverse primer comprising
(SEQ ID NO: 13)
GAGGACTCACCAATATCACACAA;
the DENV1 probe comprising
(SEQ ID NO: 12)
ACCTATGGATGGAACCTAGTAAAGCT;
the DENV3 forward primer comprising
(SEQ ID NO: 14)
GCTCAGCCTCCTCCATGATAAATG;
the DENV3 reverse primer comprising
(SEQ ID NO: 17)
GGGTGTCCTGGTGTCCACTTTCTC;
the DENV3 first probe comprising
(SEQ ID NO: 15)
CATGGTGACACAGATGGCAATGAC;
the DENV3 second probe comprising
(SEQ ID NO: 16)
CACGGTGACACAGATGGCAATGAC;
the CHIKV forward primer comprising
(SEQ ID NO: 18)
GGCGCCTACTGCTTCTGCGAC;
the CHIKV reverse primer comprising
(SEQ ID NO: 21)
TTGGTAAAGGACGCGGAGCTTAGC;
the CHIKV first probe comprising
(SEQ ID NO: 19)
AGCGAAGCACATGTGGAGAAGTCC;
the CHIKV second probe comprising
(SEQ ID NO: 20)
AGCGAAGCACACGTGGAGAAGTCC;
the DENV2 forward primer comprising
(SEQ ID NO: 22)
ACACAGATGGCAATGACAGACACG;
the DENV2 reverse primer comprising
(SEQ ID NO: 24)
CCAAGGCTGCATTGCTTCTCAC;
the DENV2 probe comprising
(SEQ ID NO: 23)
TGGAAAGAACTAGGAAAGAAAAAGACAC;
the DENV4 first forward primer comprising
(SEQ ID NO: 25)
TGGTTAGACCACCTTTCAATATG;
the DENV4 second forward primer comprising
(SEQ ID NO: 26)
TGGCTAGACCACCTTTCAATATG;
the DENV4 reverse primer comprising
(SEQ ID NO: 28)
TGCTAGCACCATCCGTAA;
and
the DENV4 probe comprising
(SEQ ID NO: 27)
CCTCAAGGGTTGGTGAAGAGATTC.

The targeted region of the viruses' or IC's RNA are transcribed into complimentary DNA (cDNA) and amplified by their respective primers in the polymerase chain reaction (PCR) respectively. The fluorophore-labelled probes then anneal to amplified DNA fragments and the fluorescent signal intensity is monitored by the amplification instrument during each PCR cycle. Target amplification is recorded as an increase and accumulation of fluorescence over time in contrast to background signal.

Thermal cycler conditions for SIgN assay are as follows:
Stage 1: 50° C. for 20 minutes
Stage 2: 95° C. for 2 minutes
Stage 3: 95° C. for 45 seconds
60° C. for 1 minute 15 seconds (acquire)
   Stage 3 performed for 45 cycles.

### Assay Controls

Virus stocks are eluted from the extraction of respective virus stocks as follows:

| Viruses | Strain/Isolate | Details |
|---|---|---|
| Chikungunya virus | IMT 2006 | https://doi.org/10.3201/ |
| | | eid1210.060596 |
| Dengue 1 virus | DEN1-16-2582 | MF314188 |
| Dengue 2 virus | DEN2-16-2447 | MF314189 |
| Dengue 3 virus | 05K4648DK1 | EU081225 |
| Dengue 4 virus | 8976/95 | AY762085 |
| Zika virus | H/PF/2013 | https://doi.org/10-1128/ |
| | | genomeA.00500-14 |

Note:
Quantified virus stocks were serial diluted. 1E5 to 1E1 copies (1 μL) were used.

Commercially available RNA controls were purchased. Traceable and standardised positive material (respective) for all six pathogen and other 3 flaviviruses are as follows:

| Vircell RNA | | Stock | | | |
|---|---|---|---|---|---|
| controls | Cat # | (copies/mL) | NCBI | Strain/Isolate | Year |
| Dengue 1 | MBC055 | 16.5 | KM204119 | Hawaii | 1944 |
| virus | | | | | |
| Dengue 2 | MBC056 | 15.5 | KM204118 | New Guinea | 1944 |
| virus | | | DEN2-16- | C | |
| | | | 2447 | From NPHL | |
| Dengue 3 | MBC057 | 17.5 | KU050695 | Philippines | 1956 |
| virus | | | | H87 | |
| Dengue 4 | MBC058 | 12.5 | KR011349 | Philippines | 1956 |
| virus | | | | H241 | |
| Chikungunya | MBC099 | 15.5 | NC_004162.2 | S27-African | 1953 |
| virus | | | | | |
| Zika virus | MBC072 | 13.0 | KX377335.1 | MR-766 | 1947 |
| Zika virus | MBC103 | 16.0 | KU501215 | PRVABC59 | 2015 |
| (Asian) | | | | | |
| St. Louis | MBC101 | 15.0 | NA | NA | NA |
| Encephalitis | | | | | |
| Virus | | | | | |
| West Nile | MBC069 | 13.0 | HQ596519.1 | New York 99 | 1999 |
| Virus | | | | | |
| Yellow Fever | MBC100 | 17.0 | FJ654700.1 | 17D-Tiantan | NA |
| Virus | | | | | |

Healthy control (HC): whole blood from healthy donor as an extraction control and positive control for the IC Primer and probe set (IC).
   HC should generate negative results with DENV, CHIKV, and ZIKV primer and probe sets, but positive results for IC;
   In LoD tests, HC was added in addition to respective vircell RNA controls (Refer to LoD in results).
No Template Control (NTC)
   NTC reactions include PCR-grade water in place of specimen;
   The NTC is a control for contamination or improper function of assay reagents resulting in false positive results.

### Human Specimens

| Human | | |
|---|---|---|
| specimens | Cohort/Year | Published study (if applicable) |
| CHIKV, | SG 2008 cohort | https://doi.org/10.1093/infdis/jiq042 |
| n = 10 | | |
| ZIKV, | SG 2016 cohort | https://doi.org/10.1093/infdis/jix276 |
| n = 10 | | |
| DENV1 | Compiled from | https://doi.org/10.1093/infdis/jix276 |
| to 4, | different sources | https://doi.org/10.1093/infdis/jiw494 |
| n = 4 each | | https://doi.org/10.1371/journal.pntd.000 |
| | | 3043 |
| | | Unpublished data |
| HC, n = 26 | extracted 2017 | https://doi.org/10.1093/infdis/jix276 |
| HC, n = 5 | extracted 2012 | https://doi.org/10.1371/journal.pntd.000 |
| | | 304 |

Comparator tests for these samples have been described in the respective publications. Additional tests that were conducted on these samples during time of collection were also described. (Please refer to respective publications for more details).

### CDC Single-Plex Assays Workflow

The performance of SIgN-DxD primers and probes under single-plex conditions were compared to the following CDC assays (reference tests) to access their performances.

Protocols were adapted from the following publications:

CHIKV: Pastorino, B. et al., Development of a TaqMan® RT-PCR assay without RNA extraction step for the detection and quantification of African Chikungunya viruses; J. Virological Methods; Vol. 124, issues 1-2, March 2006, pages 65-71;
DENV: Pastorino, B. et al., Development of a TaqMan® RT-PCR assay without RNA extraction step for the detection and quantification of African Chikungunya viruses; J. Virological Methods; Vol. 124, issues 1-2, March 2006, pages 65-71; and
ZIKV: Lanciotti et al., Genetic and Serologic Properties of Zika Virus Associated with an Epidemic, Yap State, Micronesia, 2007; EID Journal, Vol, 14, No. 8, August, 2008.

### CDC Single-Plex Reaction Set Up

| Reaction | | Volume for 1x |
|---|---|---|
| components | Stock | (μl) |
| SSH1 mastermix | 2x | 12.5 |
| Forward + | 10 μM | 1.00 |
| Reverse primers | | |
| Probe | 10 μM | 0.50 |
| RT enzyme mix | | 0.50 |
| RNA template | | 5.00 |
| Nuclease free | | 5.50 |
| H₂O | | |

Thermal cycler conditions for single-plex assay are as follows:
Stage 1: 50° C. for 20 minutes
Stage 2: 95° C. for 2 minutes
Stage 3: 95° C. for 15 seconds
60° C. for 1 minute (acquire)
   Stage 3 performed for 45 cycles.

### Data Interpretation

The threshold is normally set by the software manager using auto settings. Each amplification curve (if any) and corresponding threshold in every channel was inspected manually for confirmation. In some instances, the threshold for each channel was set at a specific value. Values shown below were set based on the background signals observed from the LoD and cross reactivity runs:
FAM channel: 90
Hex channel: 36
TxR channel: 25
Cy5 channel: 50

The IC for each specimen should always be positive. If both the IC for a specimen sample the other samples in the 2-tube assay are negative, the following steps were taken:
Repeat RT-PCR test of specimen.
Repeat extraction from new specimen aliquot.

If the IC for a specimen sample is negative, but DENV, CHIKV, and/or ZIKV is positive for specimen samples:

RT-PCR test was not repeated and consider the results of the multiplex assay valid.

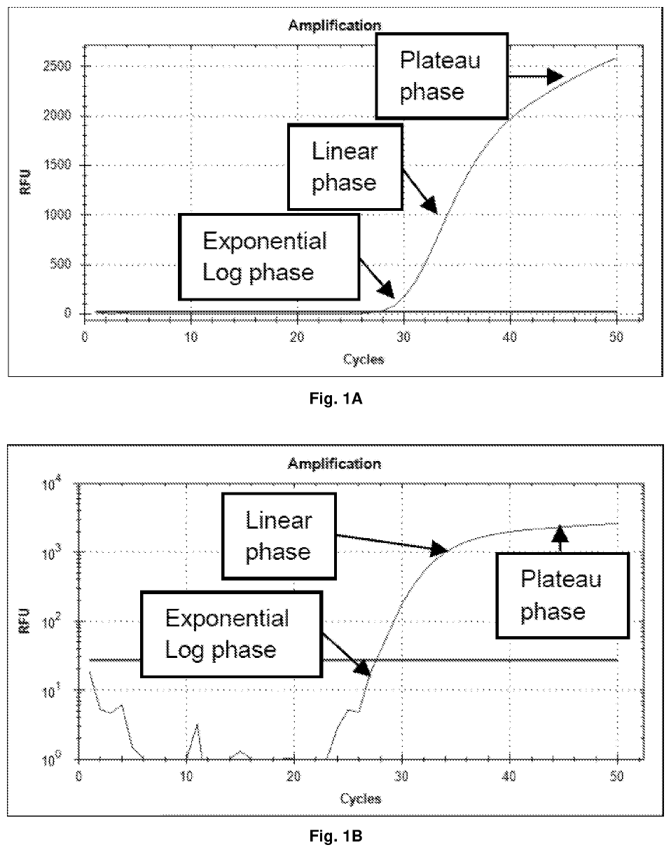

FIG. 1 illustrates generic examples of stages of PCR amplification plots in linear and log views.

True positives should produce exponential curves with logarithmic, linear, and plateau phases. (Note: Weak positives will produce high CT values that are sometimes devoid of a plateau phase; however, the exponential plot will be seen.)

For a sample to be a true positive, the curve must cross the threshold in a similar fashion as shown in FIG. 1. It must NOT cross the threshold and then dive back below the threshold.

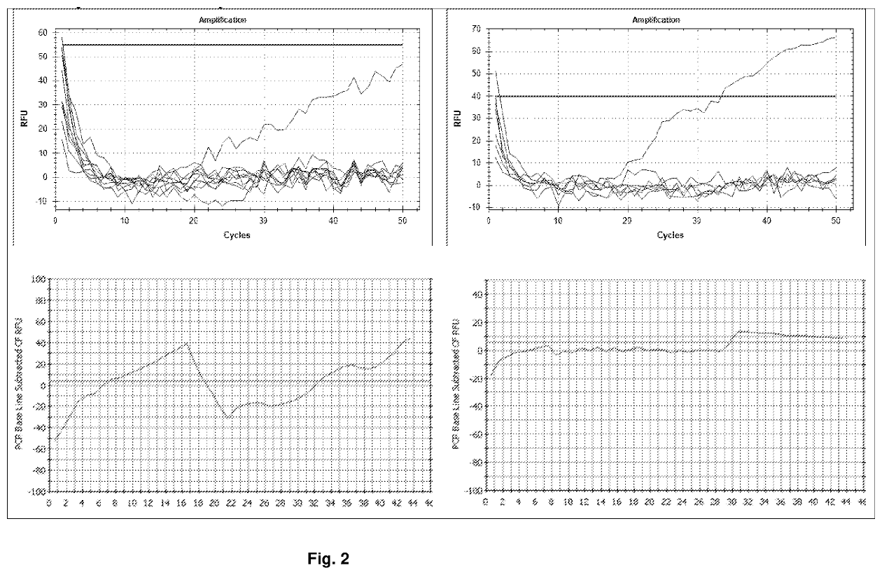

Examples of false positive curves can be found in FIG. 2.

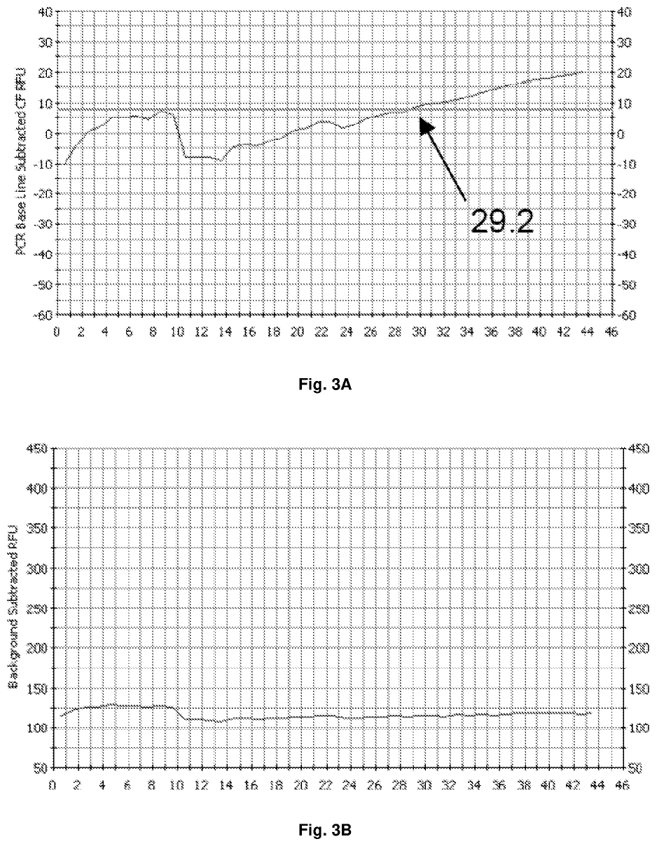

In certain situations, a low ct value (eg. ct of 29.2 in FIG. 3) might indicate a positive result. However, on manual inspection of the curve, it was evident that the sample is negative by looking at its shape and the background fluorescence view.

A note on weak positive samples: Weak positives were interpreted with caution. If curves are true exponential curves, the reaction should be interpreted as positive.

If repeat testing of a weak specimen was necessary, repeat the sample in replicates as a single repeat test run has a high likelihood of generating a discrepant result. The repeat testing should be conducted in single-plex using only primer/probe set(s) giving the weak positive signal. If it is possible to repeat the extraction of RNA from the biological specimen, eluting in a lower volume to concentrate the sample is recommended.

### Results and Discussion

### Performance of SIgN-DxD Primers and Probes Under Single-Plex Conditions

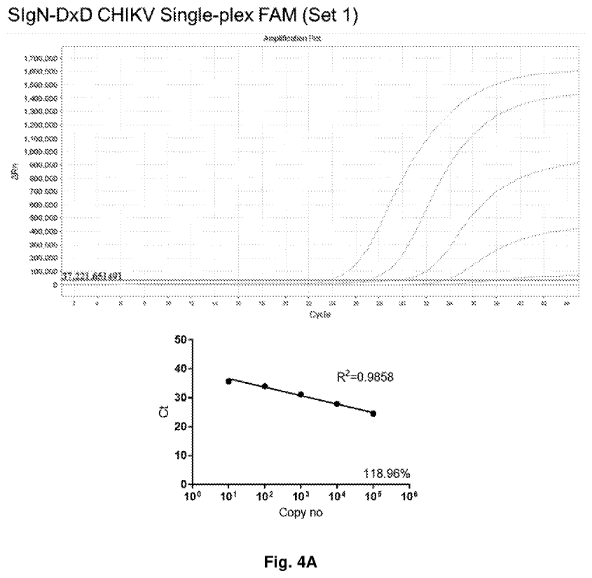

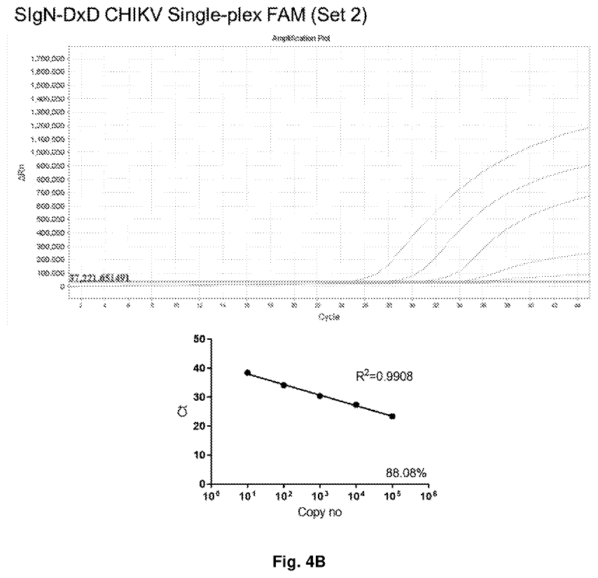

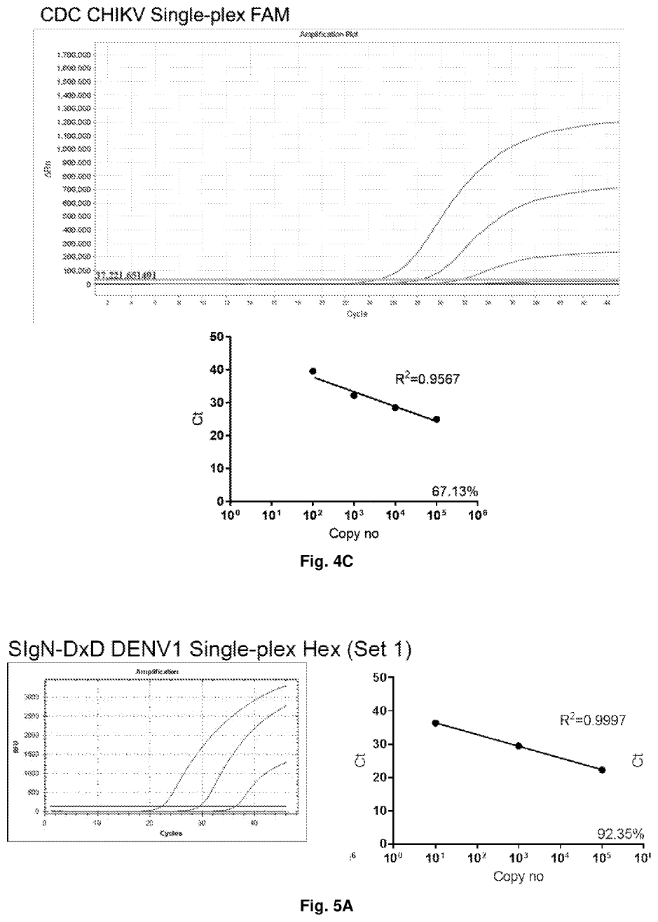

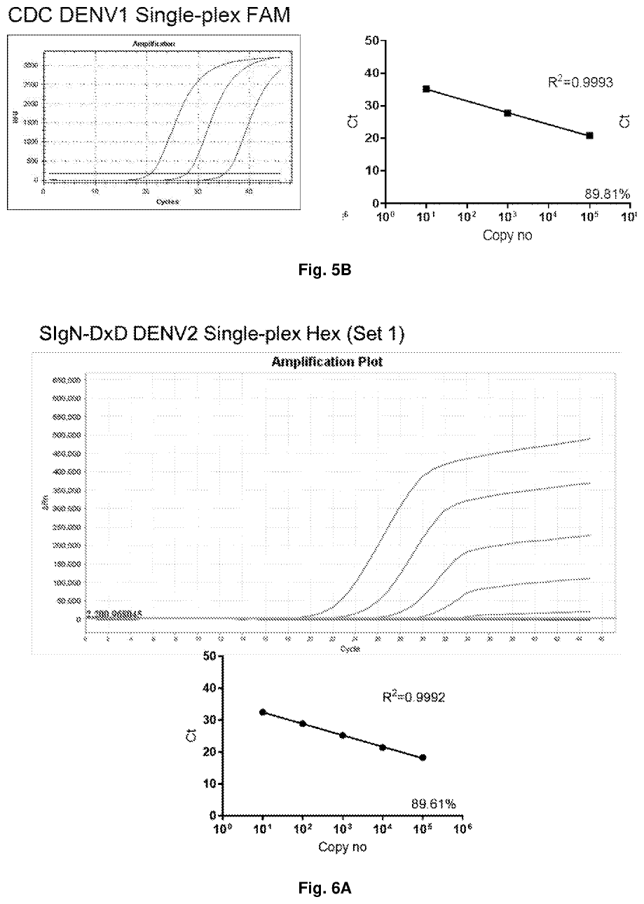

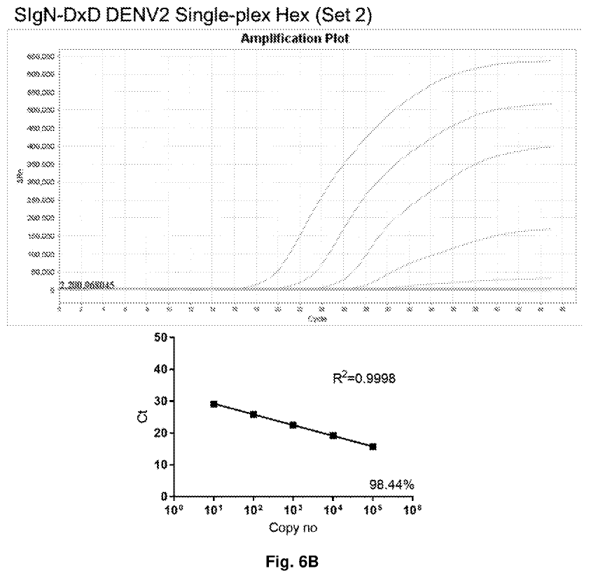

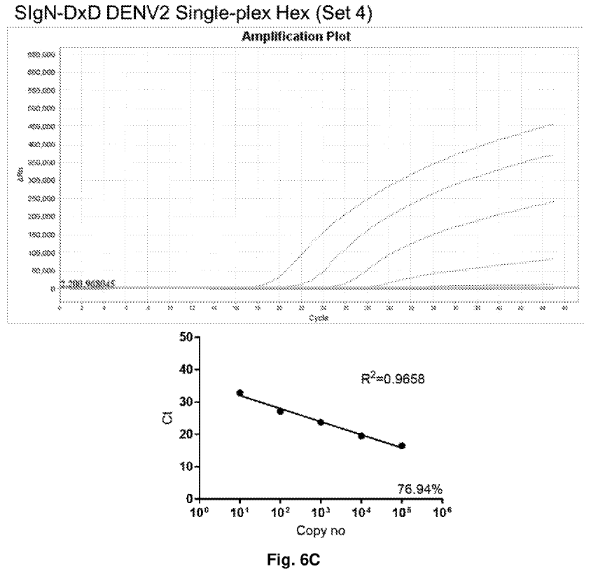

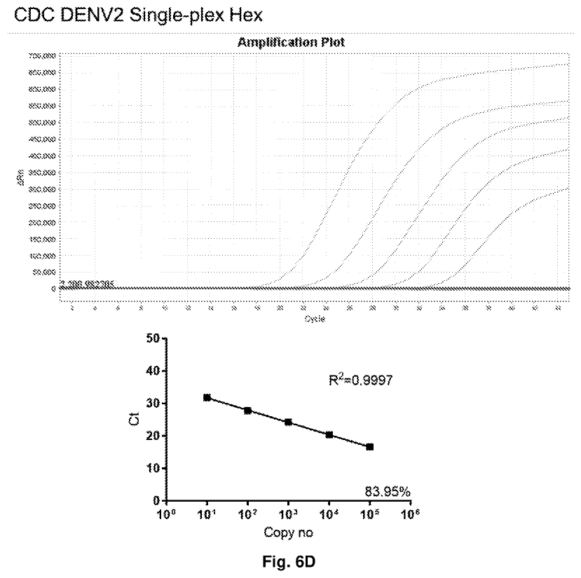

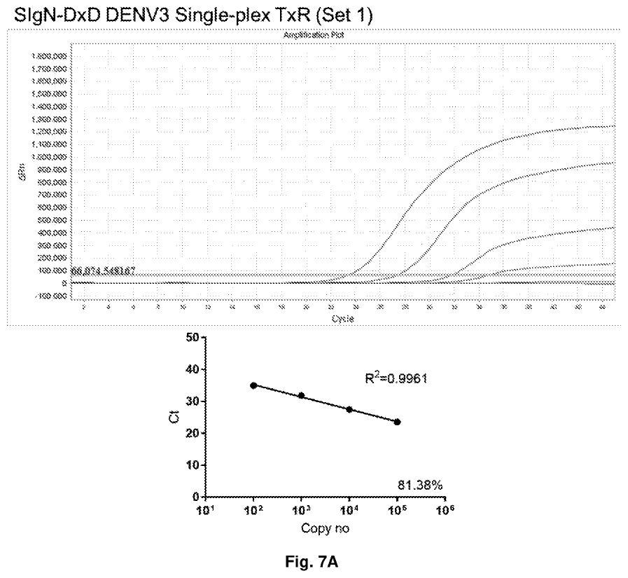

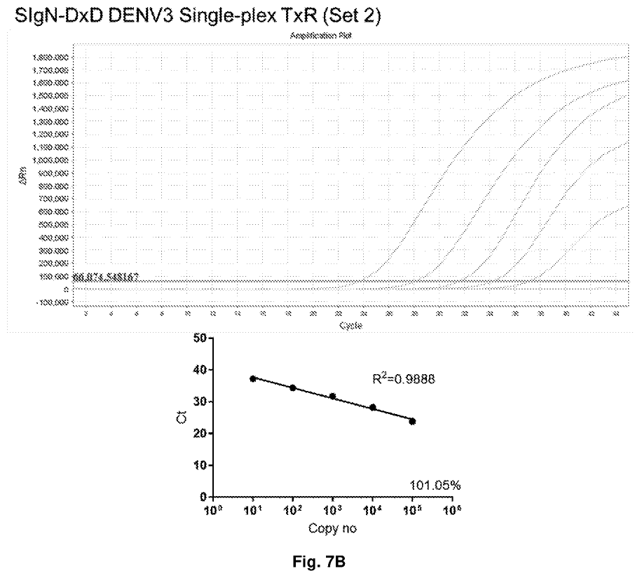

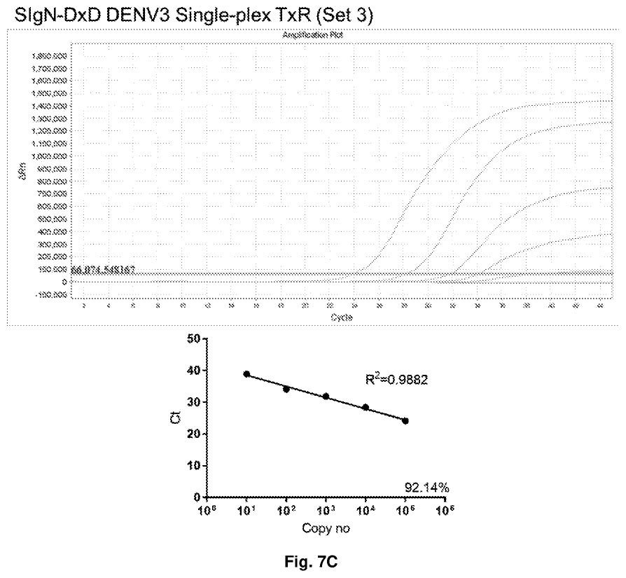

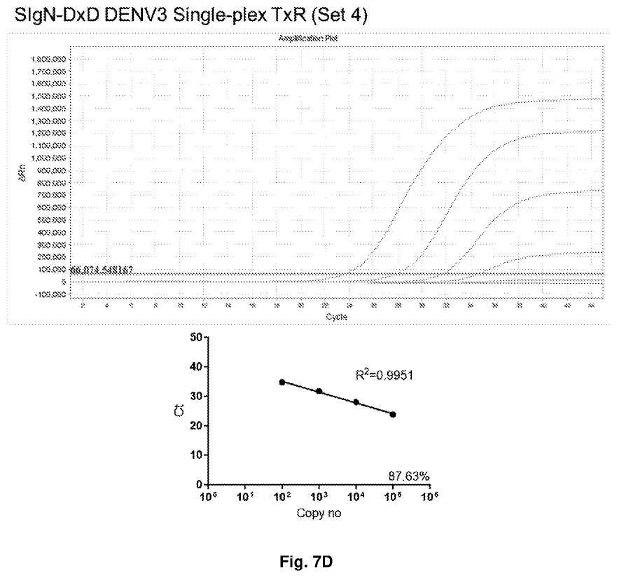

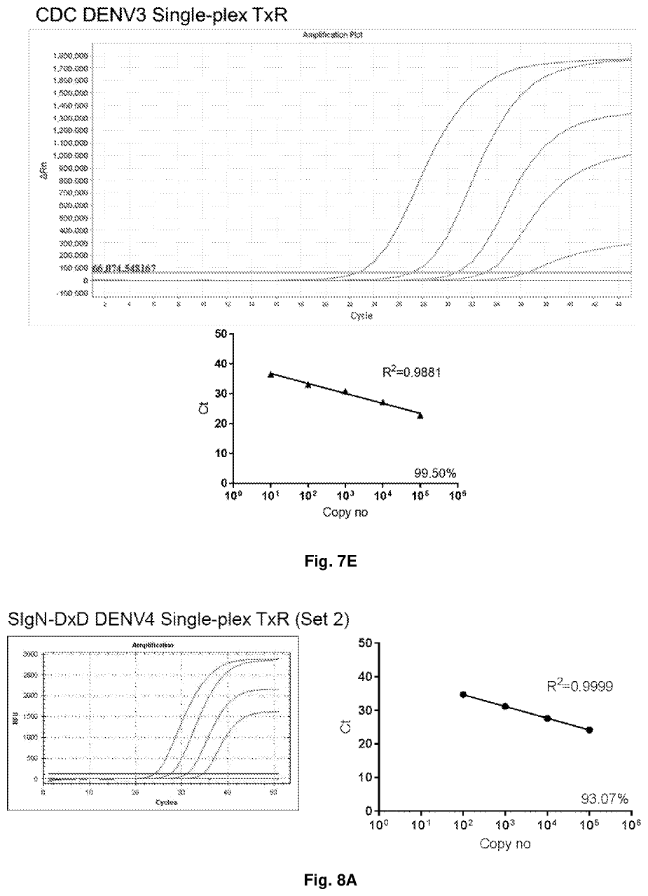

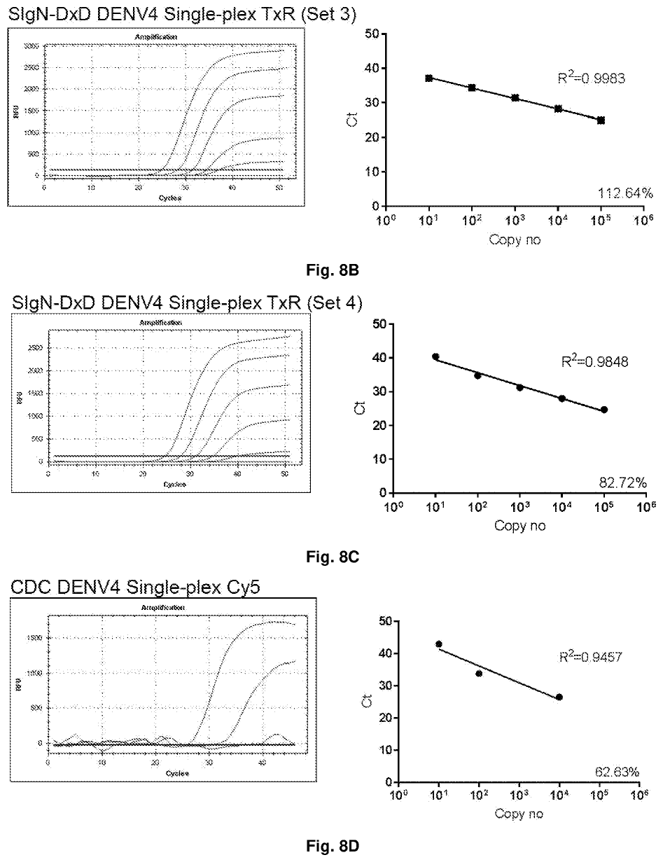

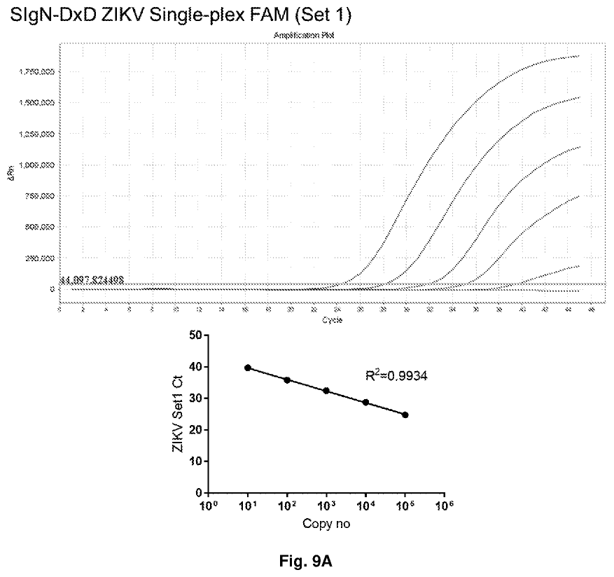

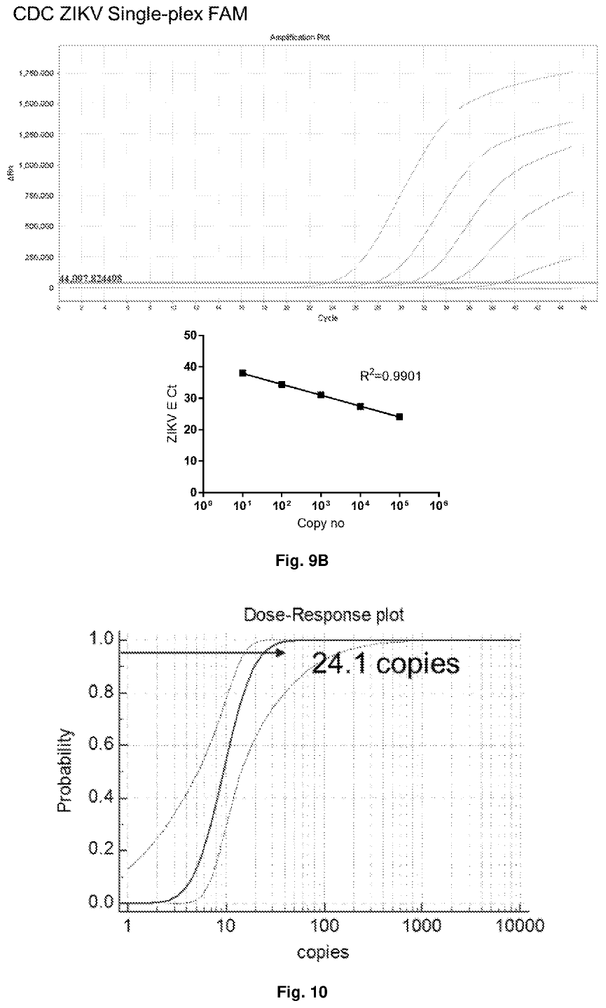

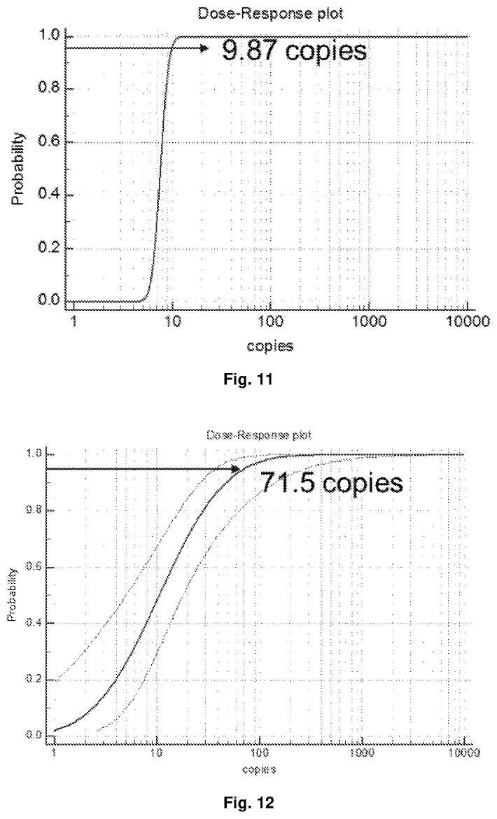

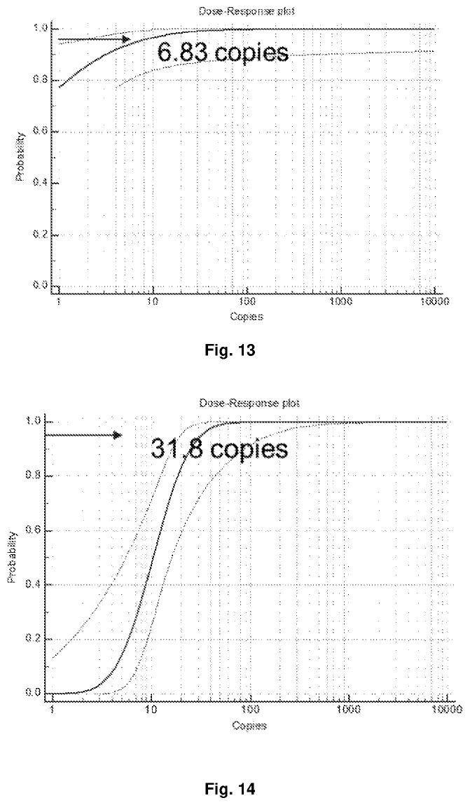

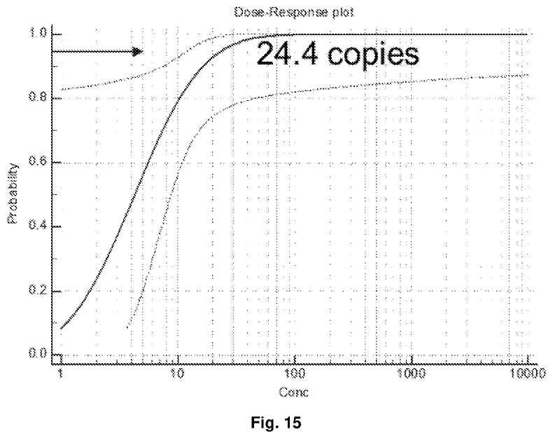

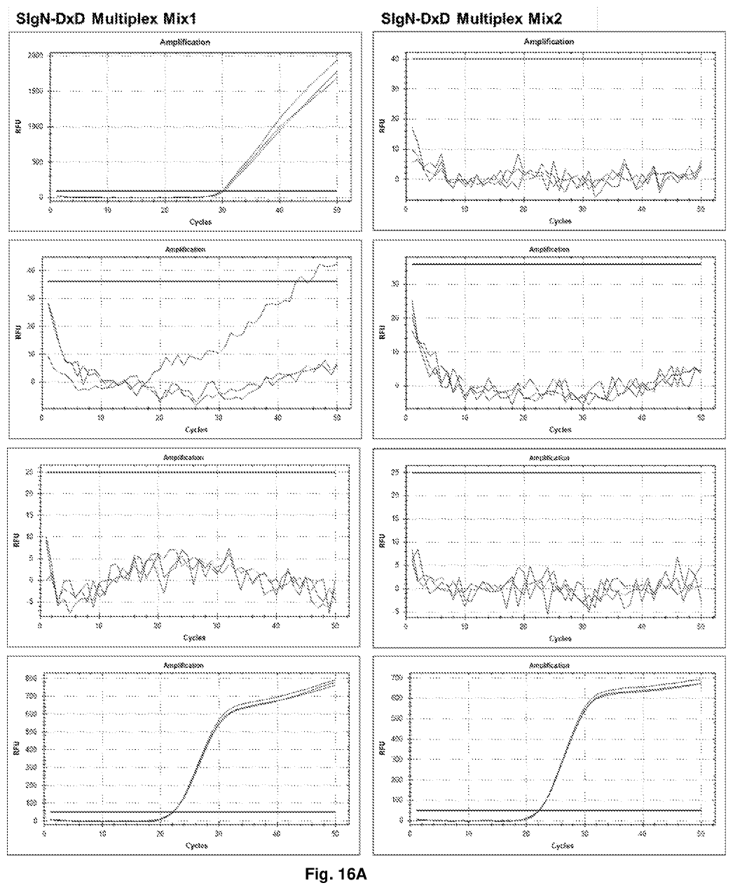

The sensitivity and specificity of SIgN-DxD primers and probes under single-plex conditions were determined in comparison to the CDC reference-PCR. For CHIKV and all four serotypes of DENV, the PCR efficiencies of the SIgN-DXD PCR (see FIG. 4A and 4B for CHIKV; FIG. 5A for DENV1; FIG. 6A, FIG. 6B, and FIG. 6C for DENV2; FIG. 7A, FIG. 7B, FIG. 7C and FIG. 7D for DENV3; FIG. 8A, FIG. 8B, FIG. 8C for DENV4; and FIG. 9A for ZIKV) were higher than that of the CDC PCR (see FIG. 4C for CHIKV; FIG. 5B for DENV1; FIG. 6D for DENV2; FIG. 7E for DENV3; FIG. 8D for DENV4; and FIG. 9B for ZIKV). The results are summarized in tables below:

TABLE 1.1
Summary of PCR efficiency in single-plex conditions

| Target | SIgN | CDC |
|---|---|---|
| CHIKV | 100% | 67.13% |
| DENV 1 | 92.35% | 89.81% |
| DENV 2 | 98.44 | 83.95% |
| DENV 3 | 100% | 99.5% |
| DENV 4 | 100% | 62.63% |
| ZIKV | 86.86% | 93.99% |

TABLE 1.2
Comparison of SIgN-DxD CHIKV PCR with CDC CHIKV PCR

| SIgN-DxD CHIKV | | | |
|---|---|---|---|
| Single-plex Set (1) | | CDC CHIKV Single-plex | |
| Copy No | Ct | Copy No | Ct |
| 1E5 | 24.83 | 1E5 | 24.90 |
| 1E4 | 27.96 | 1E4 | 28.52 |
| 1E3 | 30.97 | 1E3 | 32.45 |
| 1E2 | 34.02 | 1E2 | 39.56 |
| 1E1 | 35.69 | 1E1 | ND |
| PCR Efficiency | 100% | PCR Efficiency | 67.13% |

TABLE 1.3
Comparison of SIgN-DxD DENV1 PCR with CDC DENV1 PCR

| SIgN-DxD DENV1 Single-plex (Set 1) | | CDC DENV1 Single-plex | |
|---|---|---|---|
| 1E5 | 22.29 | 1E5 | 20.83 |
| 1E3 | 29.53 | 1E3 | 27.68 |
| 1E1 | 36.37 | 1E1 | 35.20 |
| PCR Efficiency | 92.35% | PCR Efficiency | 89.81% |

TABLE 1.4
Comparison of SIgN-DxD DENV2 PCR with CDC DENV2 PCR

| SlgN-DxD DENV2 Single-plex Set (2) | | CDC DENV2 Single-plex | |
|---|---|---|---|
| Copy No | Ct | Copy No | Ct |
| 1E5 | 15.7 | 1E5 | 16.64 |
| 1E4 | 19.17 | 1E4 | 20.38 |
| 1E3 | 22.57 | 1E3 | 24.26 |
| 1E2 | 25.85 | 1E2 | 27.78 |
| 1E1 | 29.15 | 1E1 | 31.83 |
| PCR Efficiency | 98.44% | PCR Efficiency | 83.95% |

TABLE 1.5
Comparison of SlgN-DxD DENV3 PCR with CDC DENV3 PCR

| SlgN-DxD DENV3 Single-plex Set (2) | | CDC DENV3 Single-plex | |
|---|---|---|---|
| Copy No | Ct | Copy No | Ct |
| 1E5 | 23.84 | 1E5 | 22.81 |
| 1E4 | 28.25 | 1E4 | 27.29 |
| 1E3 | 31.72 | 1E3 | 30.85 |
| 1E2 | 34.41 | 1E2 | 33.16 |
| 1E1 | 37.25 | 1E1 | 36.55 |
| PCR Efficiency | 100% | PCR Efficiency | 99.5% |

TABLE 1.6
Comparison of SlgN-DxD DENV4 PCR with CDC DENV4 PCR

| SlgN-DxD DENV4 Single-plex Set (3) | | CDC DENV4 Single-plex | |
|---|---|---|---|
| Copy No | Ct | Copy No | Ct |
| 1E5 | 24.95 | 1E5 | — |
| 1E4 | 28.27 | 1E4 | 26.54 |
| 1E3 | 31.45 | 1E3 | — |
| 1E2 | 34.45 | 1E2 | 33.81 |
| 1E1 | 37.12 | 1E1 | 43.02 |
| PCR Efficiency | 100% | PCR Efficiency | 62.23% |

TABLE 1.7
Comparison of SIgN-DxD ZIKV PCR with CDC ZIKV PCR

| SIgN-DxD ZIKV Single-plex (Set 1) | | | CDC ZIKV Single-plex | | |
|---|---|---|---|---|---|
| Copy No | Ct1 | Ct2 | Copy No | Ct1 | Ct2 |
| 1.00E+05 | 24.52 | 25.06 | 1.00E+05 | 23.92 | 24.33 |
| 1.00E+04 | 28.3 | 29.27 | 1.00E+04 | 27.74 | 27.02 |
| 1.00E+03 | 32.05 | 32.82 | 1.00E+03 | 30.93 | 31.26 |
| 1.00E+02 | 35.21 | 36.34 | 1.00E+02 | 34.23 | 34.54 |
| 1.00E+01 | 39.62 | 39.8 | 1.00E+01 | 38.97 | 37.03 |

| | | | |
|---|---|---|---|
| PCR Efficiency | 88.86% | PCR Efficiency | 93.99% |

### Limit of Detection (LoD)

The LoD of each target in the multiplex PCR is an important measure for the lowest detectable RNA copy number for each pathogen. By definition, the LoD of the multiplex PCR is determined as the lowest copy number which, in terms of RNA copy, when added to the assay, leads to a positive pathogen identification outcome more than 95% of the time. In the analysis below, the lower limit of 95% detection (95% LLOD) was calculated using probit analysis by extrapolating the probit graph at the 0.95 (y-axis) as represented by the blue arrow in the graphs below. The two red dotted lines that flank the probit graph represent the 95% confidence interval at 95% LLOD.

### SIgN-DxD Multiplex Mix1 ZIKV

TABLE 2.1
ZIKV copies and corresponding detectable Ct

| Copies | Replicates | No. with detectable Ct |
|---|---|---|
| 1 | 10 | 0 |
| 7 | 10 | 3 |
| 20 | 10 | 9 |
| 50 | 10 | 10 |
| 100 | 10 | 10 |
| 250 | 10 | 10 |
| 500 | 10 | 10 |
| 1000 | 10 | 10 |

### SIgN-DxD Multiplex Mix1 DENV1

TABLE 2.2
DENV1 copies and corresponding detectable Ct

| Copies | Replicates | No. with detectable Ct |
|---|---|---|
| 1 | 10 | 0 |
| 7 | 10 | 3 |
| 20 | 10 | 10 |
| 50 | 10 | 10 |
| 100 | 10 | 10 |
| 250 | 10 | 10 |
| 500 | 10 | 10 |
| 1000 | 10 | 10 |

### SIgN-DxD Multiplex Mix1 DENV3

TABLE 2.3
DENV2 copies and corresponding detectable Ct

| Copies | Replicates | No. with detectable Ct |
|---|---|---|
| 1 | 10 | 1 |
| 7 | 10 | 2 |
| 20 | 10 | 6 |
| 50 | 10 | 10 |
| 100 | 10 | 10 |
| 250 | 10 | 10 |
| 500 | 10 | 10 |
| 1000 | 10 | 10 |

### SIgN-DxD Multiplex Mix2 CHIKV

TABLE 2.4
CHIKV copies and corresponding detectable Ct

| Copies | Replicates | No. with detectable Ct |
|---|---|---|
| 1 | 10 | 8 |
| 7 | 10 | 9 |
| 20 | 10 | 10 |
| 50 | 10 | 10 |
| 100 | 10 | 10 |
| 250 | 10 | 10 |
| 500 | 10 | 10 |
| 1000 | 10 | 10 |

### SIgN-DxD Multiplex Mix2 DENV2

TABLE 2.5
DENV2 copies and corresponding detectable Ct

| Copies | Replicates | No. with detectable Ct |
|---|---|---|
| 1 | 10 | 0 |
| 7 | 10 | 3 |
| 20 | 10 | 8 |
| 50 | 10 | 10 |
| 100 | 10 | 10 |
| 250 | 10 | 10 |
| 500 | 10 | 10 |
| 1000 | 10 | 10 |

### SIgN-DxD Multiplex Mix2 DENV4

TABLE 2.6
DENV4 copies and corresponding detectable Ct

| Copies | Replicates | No. with detectable Ct |
|---|---|---|
| 1 | 10 | 1 |
| 7 | 10 | 7 |
| 20 | 10 | 9 |
| 50 | 10 | 10 |
| 100 | 10 | 10 |
| 250 | 10 | 10 |
| 500 | 10 | 10 |
| 1000 | 10 | 10 |

TABLE 2.7
Summary of LoDs for each of the virus targets

| Virus | 95% LLOD | 95% Confidence Interval | |
|---|---|---|---|
| Dengue 1 virus | 9.87 | NA | NA |
| Dengue 2 virus | 31.8 | 19.5 | 156.7 |
| Dengue 3 virus | 71.5 | 37.2 | 285.6 |
| Dengue 4 virus | 24.4 | 12.4 | 108.1 |
| Chikungunya | 6.83 | 1.4 | 9.18E+09 |
| virus | | | |
| Zika virus (Asian | 24.1 | 15.5 | 139.4 |
| Lineage) | | | |

### Cross-Reactivity

Evaluation of the cross-reactivity of each component of the multiplex assay with the viruses targeted by the other components was performed. Three additional flaviviruses (WNV, YFV and SLEV) were selected to evaluate the specificity of the DENV, ZIKV and CHIKV primer and probe sets. The graphs below show the amplification curve (if any) in all channels for each virus tested.

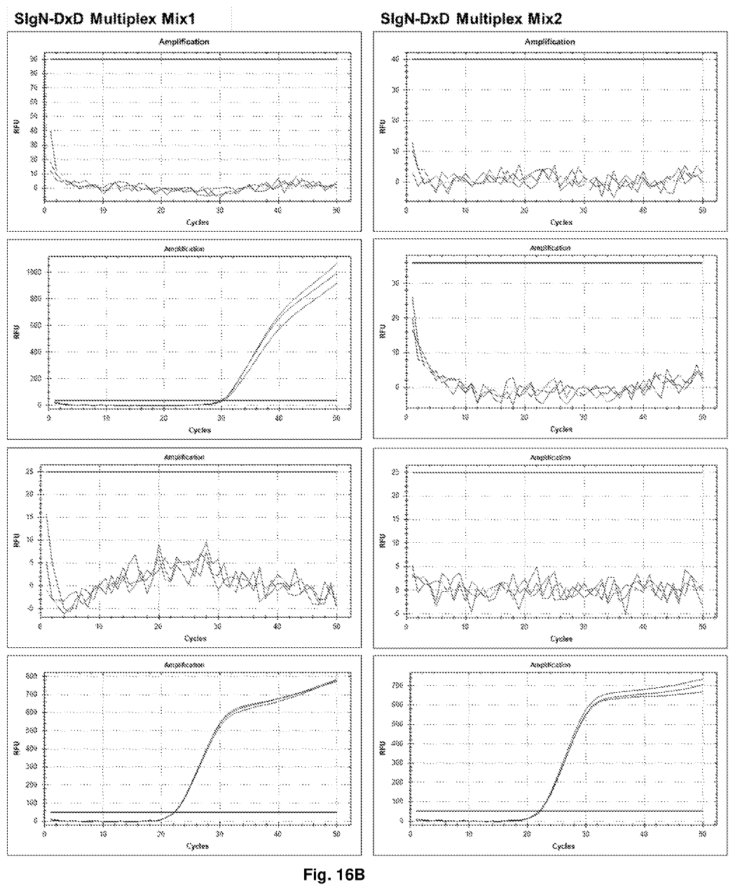

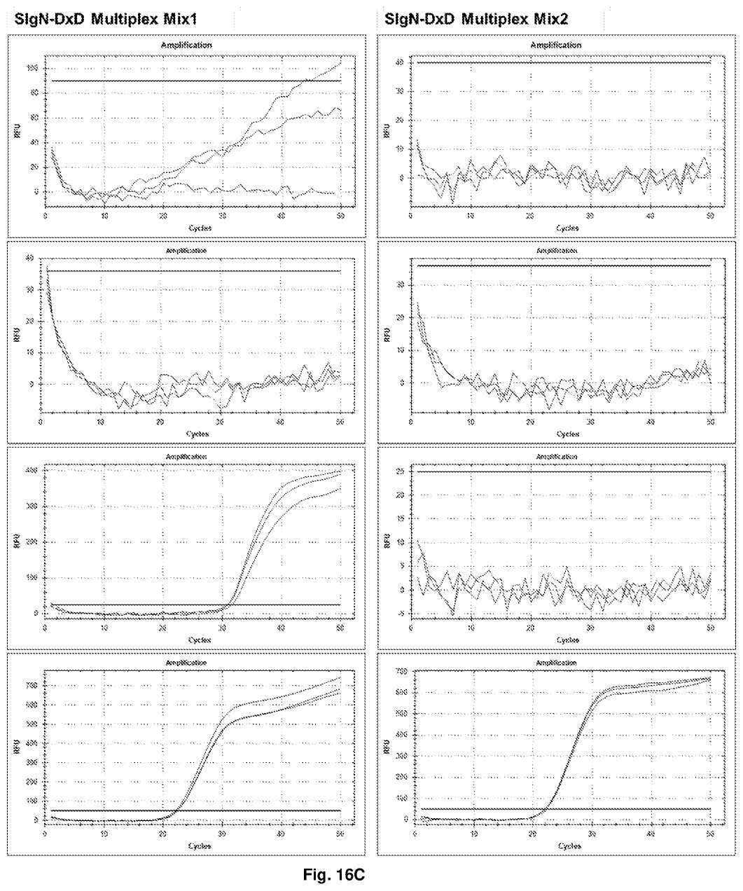

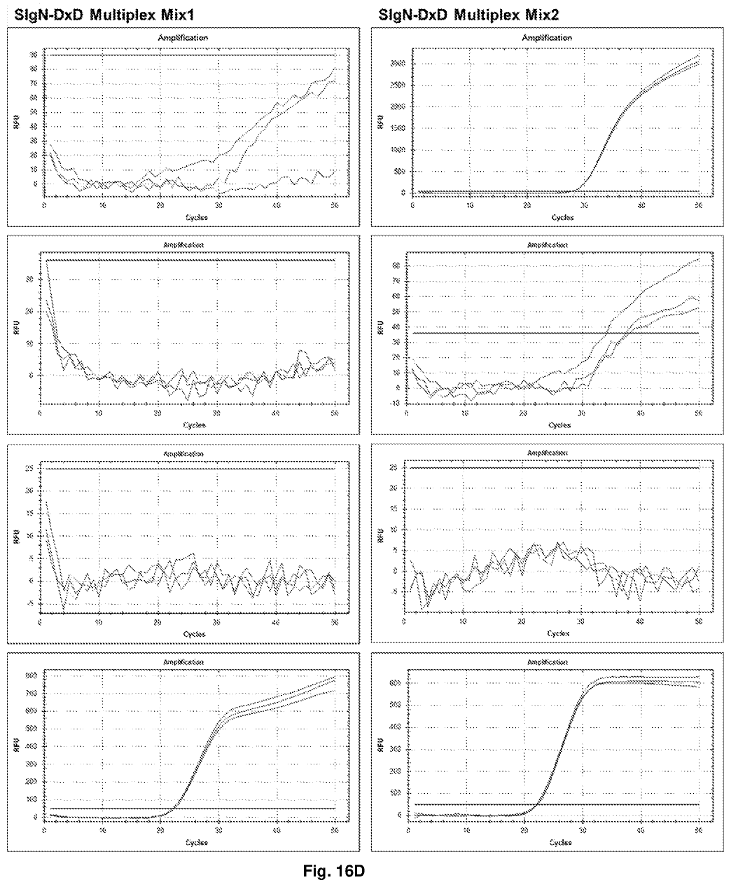

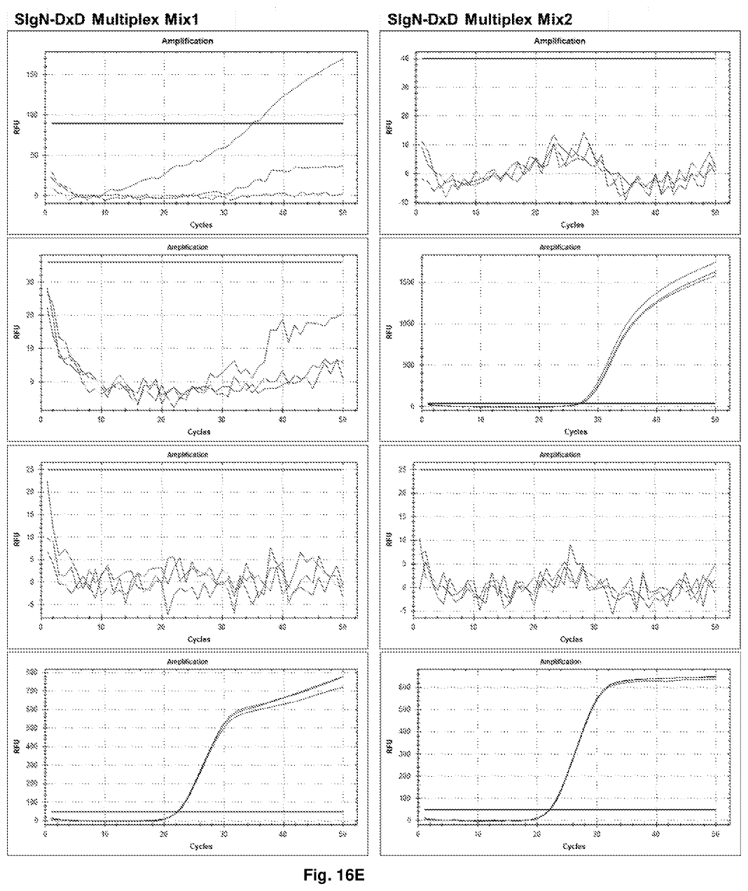

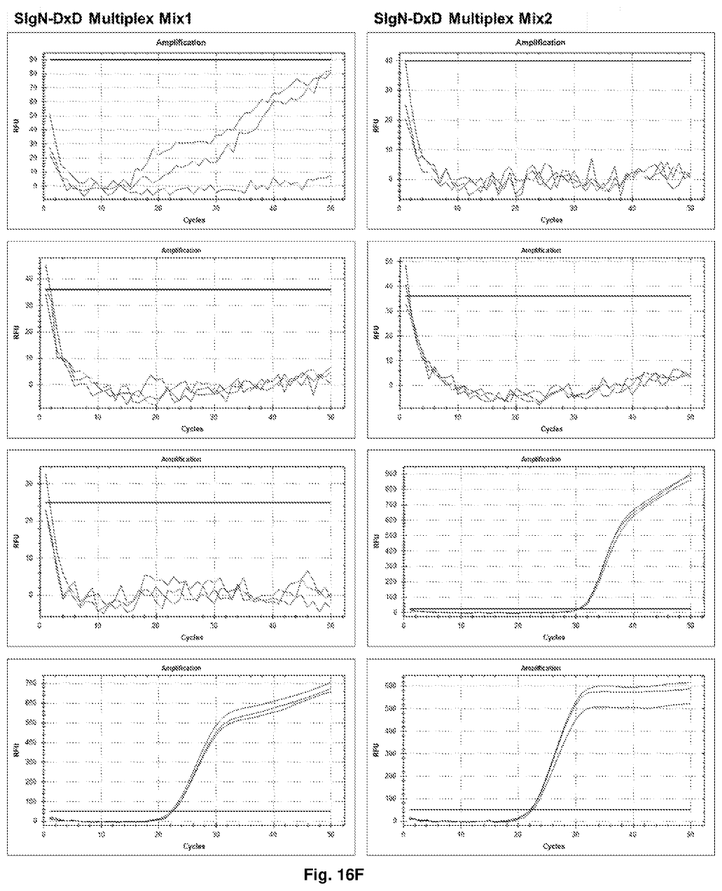

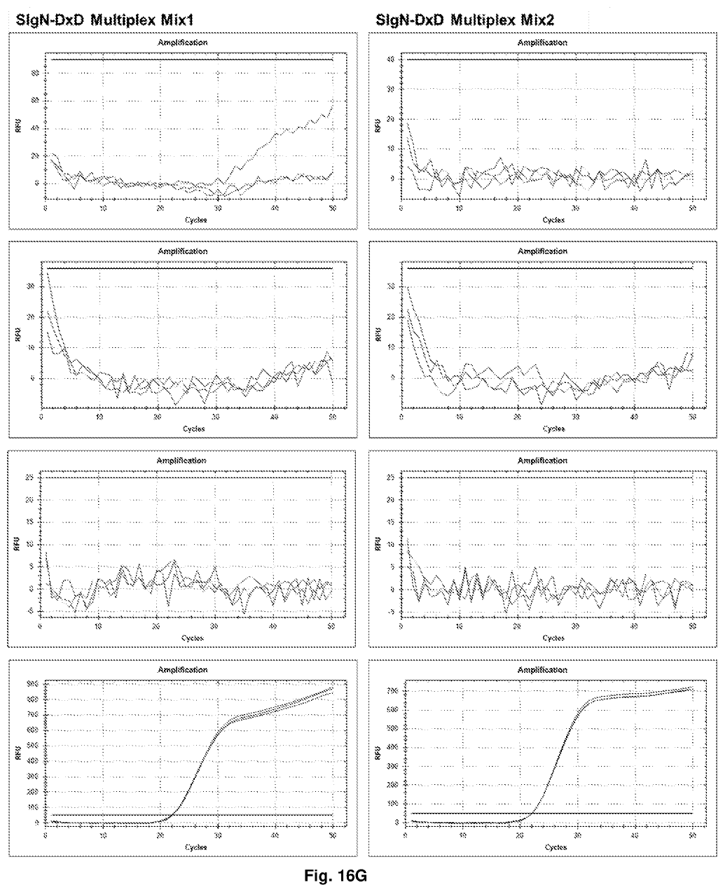

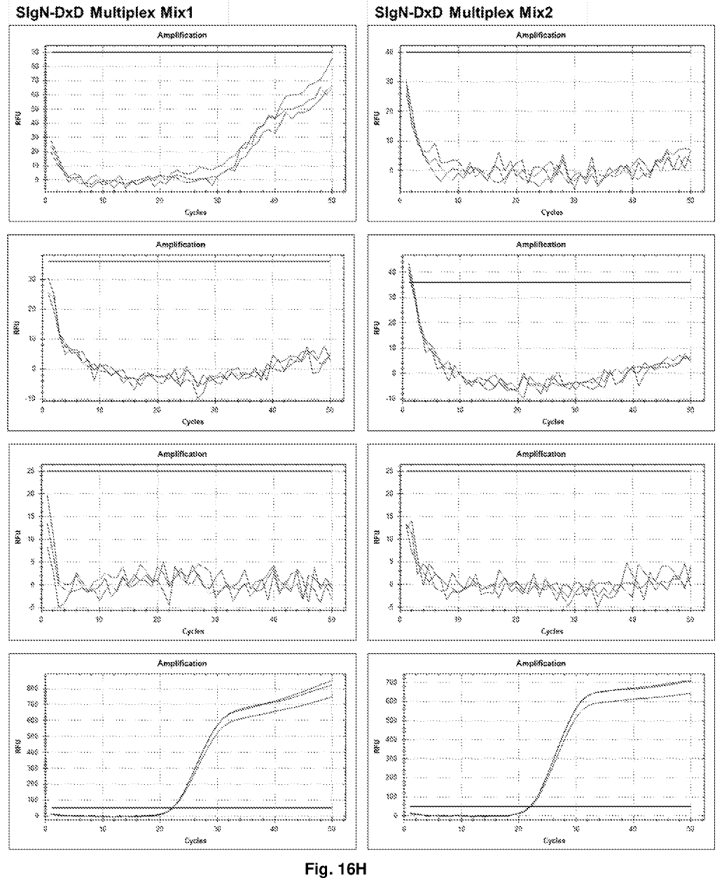

As seen in FIGS. 16A — FIG. 161, Mix1 is specific to DENV1, DENV3 and ZIKV whereas Mix2 is specific to DENV2, DENV4 and CHIKV1. No cross-reactivity was seen for these targets between the two mixes. Furthermore, as expected, SLVE, WNV and YFV were not detected by either mixes. These observations are summarised in Table 3 below.

TABLE 3
Near-neighbour Cross Reactivity Summary

| | Mix 1 | | | Mix2 | | |
|---|---|---|---|---|---|---|
| RNA | ZIKV | DENV1 | DENV3 | CHIKV | DENV2 | DENV4 |
| DENV1 | X | ✓ | X | X | X | X |
| DENV2 | X | X | X | X | ✓ | X |
| DENV3 | X | X | ✓ | X | X | X |
| DENV4 | X | X | X | X | X | ✓ |
| ZIKV | ✓ | X | X | X | X | X |
| CHIKV | X | X | X | ✓ | X | X |
| SLEV | X | X | X | X | X | X |
| WNV | X | X | X | X | X | X |
| YFV | X | X | X | X | X | X |

No cross-reactivity was observed. All controls performed as expected.

### Assay Validation on Human Specimens

In order to assess the clinical performance of the assay, the multiplex PCR was evaluated on clinical specimens to compare its diagnostic capability with reference methods. In the last set of experiments, the multiplex PCR assay was performed with RNA extracted from a few cohorts of patient samples as listed below:

### Plates Ran:

### Plate 1 (Date: 27-12-04 10-35-18) Human 1:

CHIKV, n=10
ZIKV, n=10
Healthy controls, n=6 (RNA extracted in 2017), n=5 (RNA extracted in 2012)

### Plate 2 (Date: 2017-11-21 11-36-37) Cross2:

Healthy controls, n=20 (RNA extracted in 2017)
(Rows A, C, E 1 to 12): HC1-14 ran in replicate of 3 in Mix1
(Rows B, D, F 1 to 12): HC1-14 ran in replicate of 3 in Mix2

### (Rows G7-12): HC15-20 Mix1

### (Rows H7-12): HC15-20 Mix2

### Plate 3 (Date: 2017-12-04 10-35-18) Human2:

DENV1 to 4, n=4 (each)

### ZIKV Samples

9 out of 10 ZIKV samples tested positive for ZIKV. The one sample that did not come up positive for ZIV in assay was also negative in a comparator's test. The assay is able to detect samples with low ZIKV viral load (FIG. 17A left panel) and there was no cross reactivity in other channels (Hex, TxR) within Mix 1 (FIG. 17A). No cross reactivity was seen in Mix 2 and Mix 1 and 2 IC are stable (FIG. 17A and FIG. 17B).

### DENV1 Samples

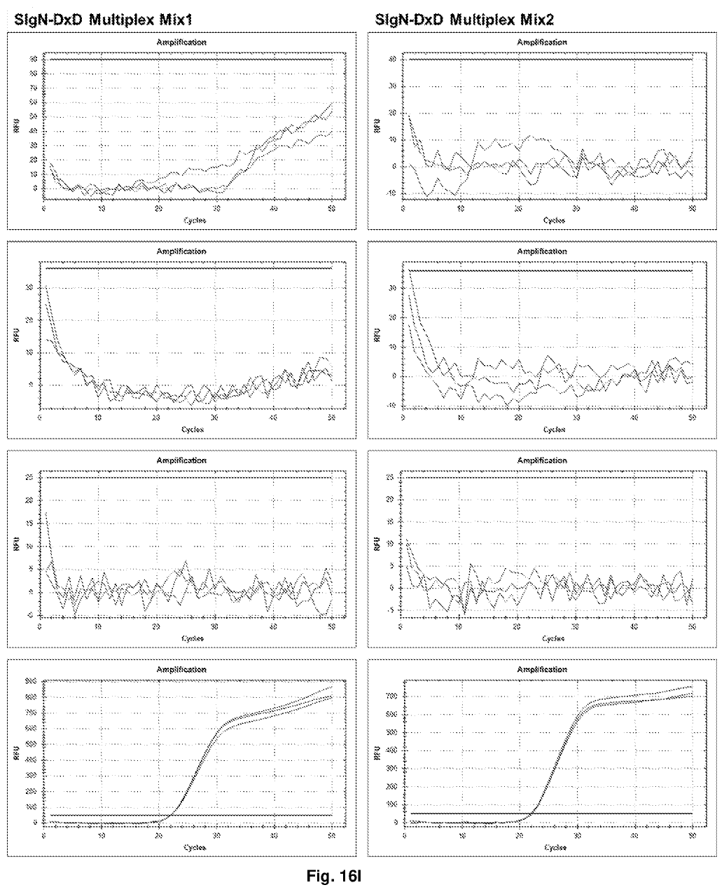

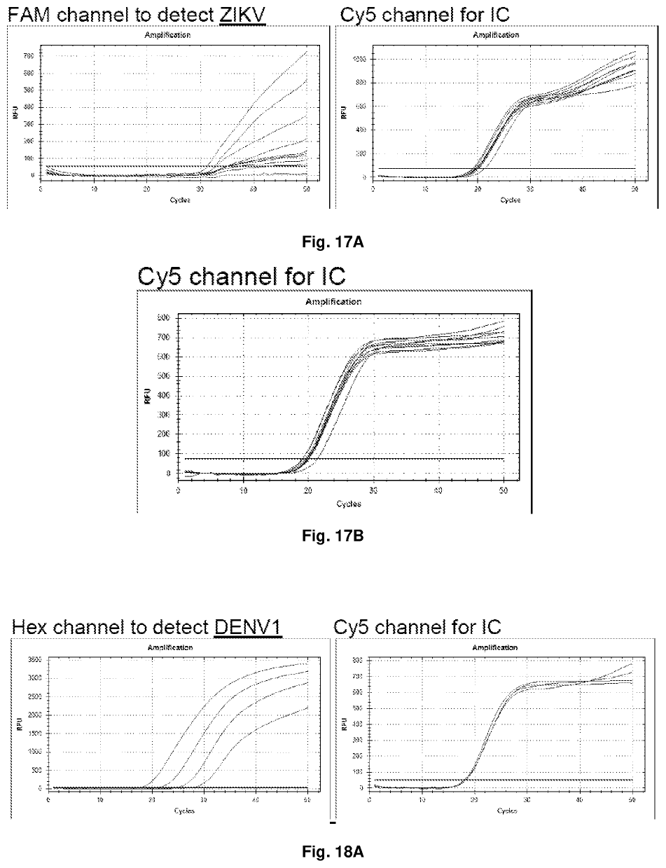

All 4 samples tested positive for DENV1 (FIG. 18A left panel) and there was no cross reactivity in other channels (FAM, TxR) within Mix 1. No cross reactivity was seen in Mix 2 and Mix 1 and 2 IC are stable (FIG. 18A and FIG. 18B).

### DENV3 Samples

All 4 samples tested positive for DENV3 (FIG. 19A left panel) and there was no cross reactivity in the other channels (FAM, Hex) within Mix 1. No cross reactivity was seen in Mix 2 and Mix 1 and 2 IC are stable (FIG. 19A and FIG. 19B).

### CHIKV Samples

All 10 samples tested positive for CHIKV. As seen from FIG. 20A on the left, the samples had a varying range of viral load and the assay was able to detect all the samples regardless of their high or low CHIKV viral load. There was no cross reactivity in other channels (Hex, TxR) within Mix 2 (FIG. 20A right panel). No cross reactivity was seen in Mix 1 and Mix2 IC are stable (FIG. 20B).

### DENV2 Samples

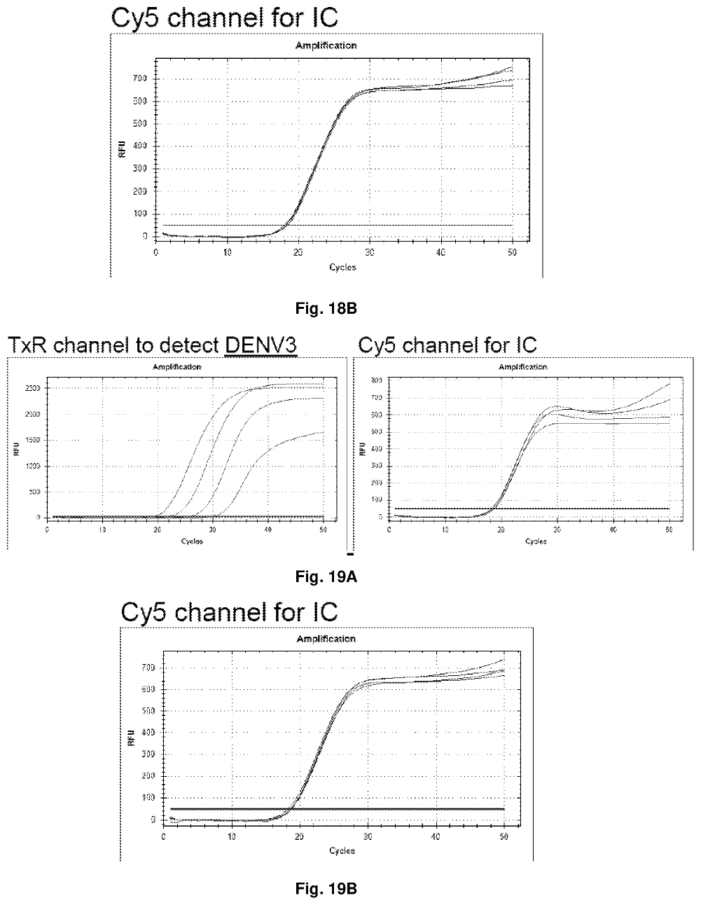

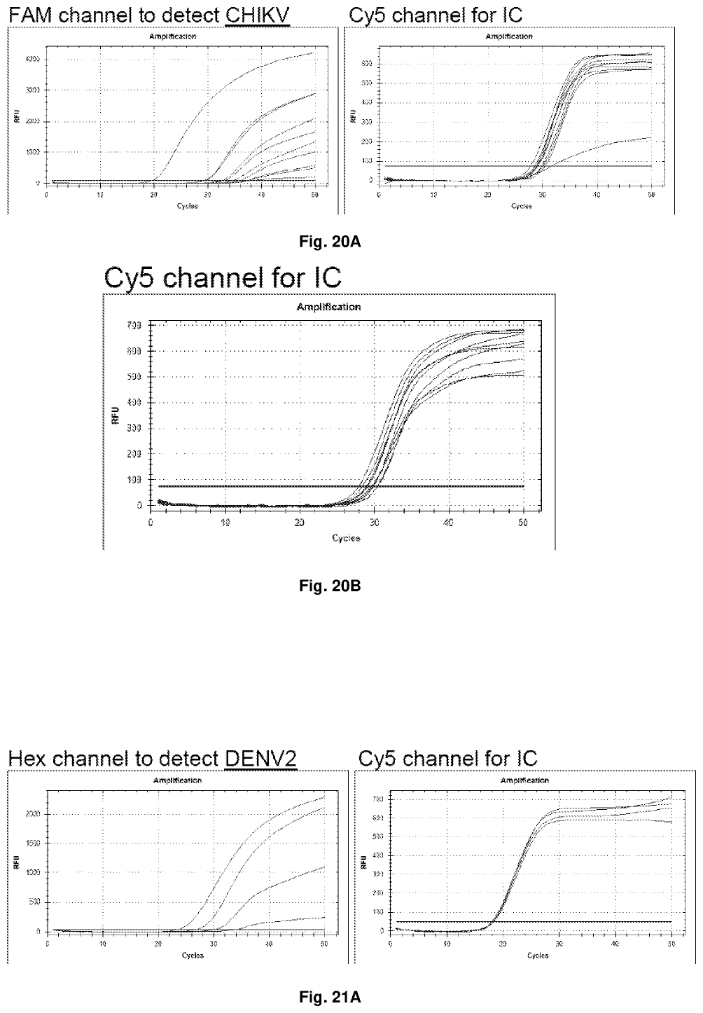

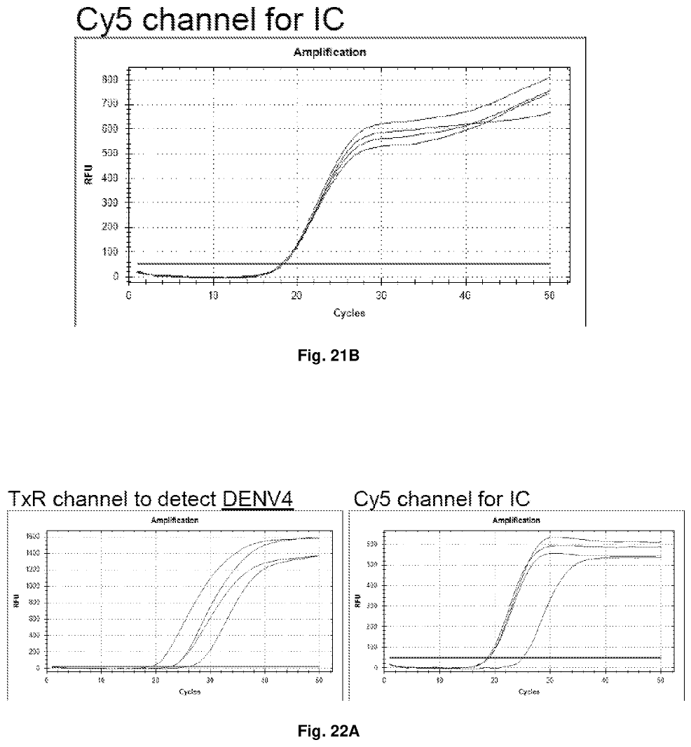

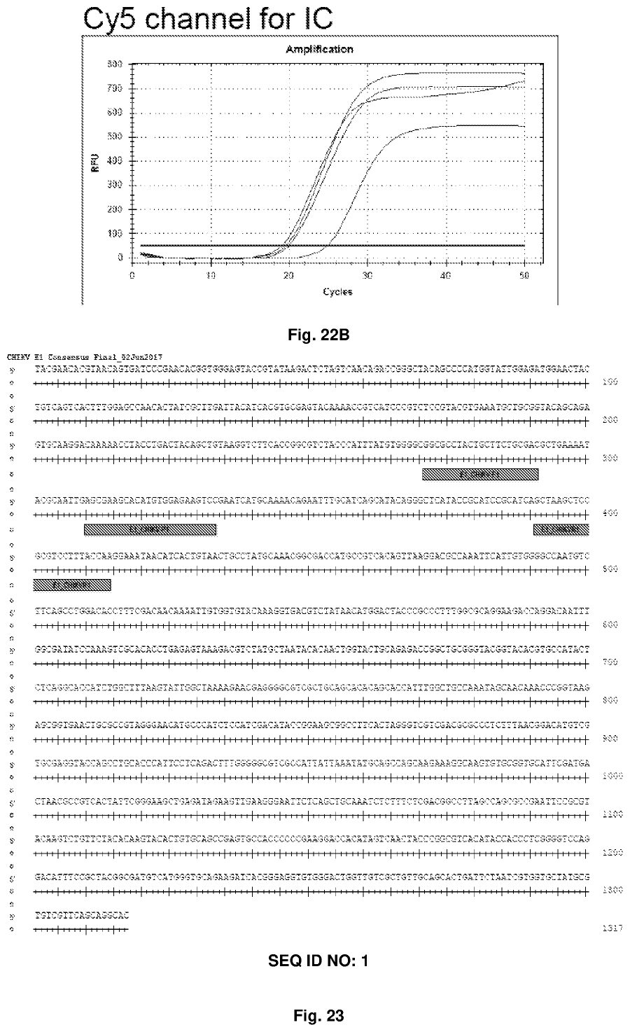

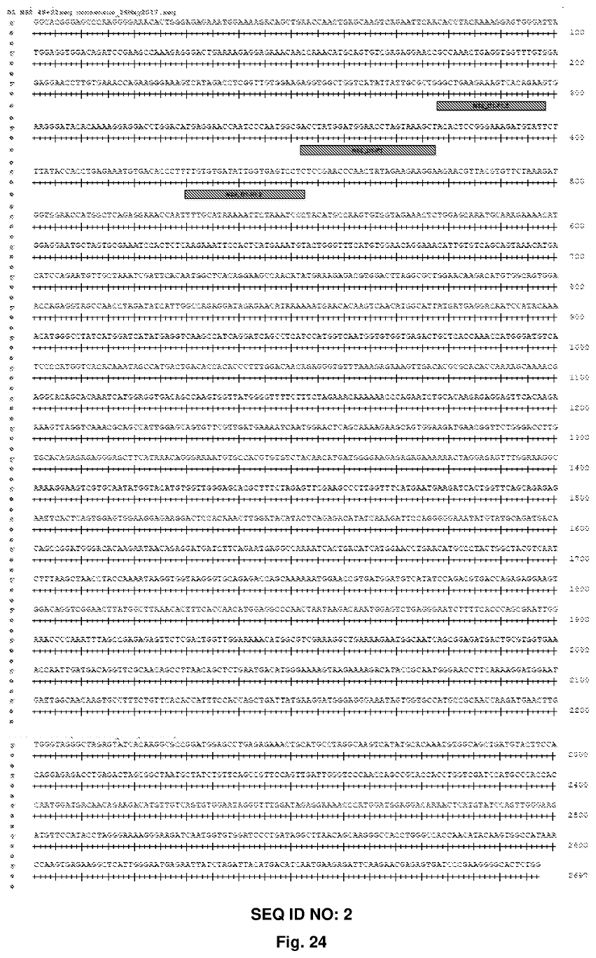

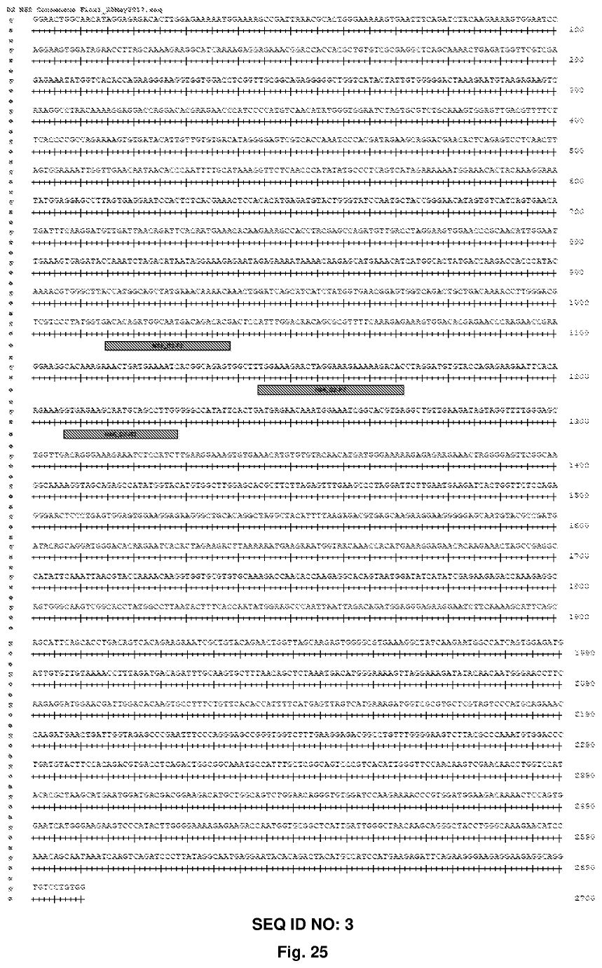

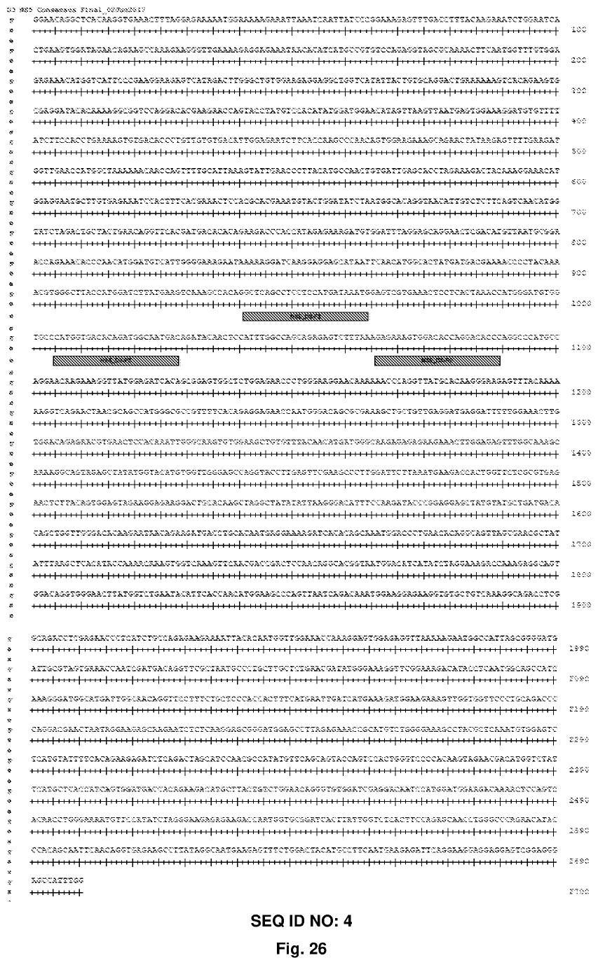

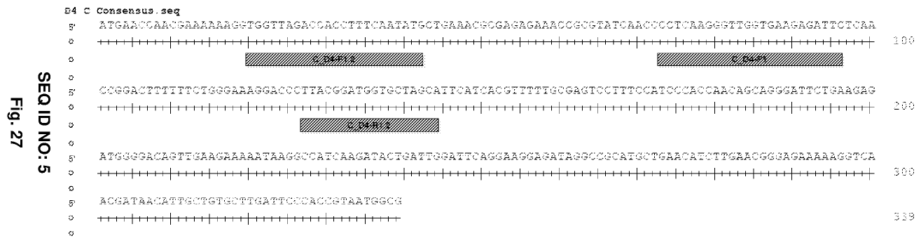

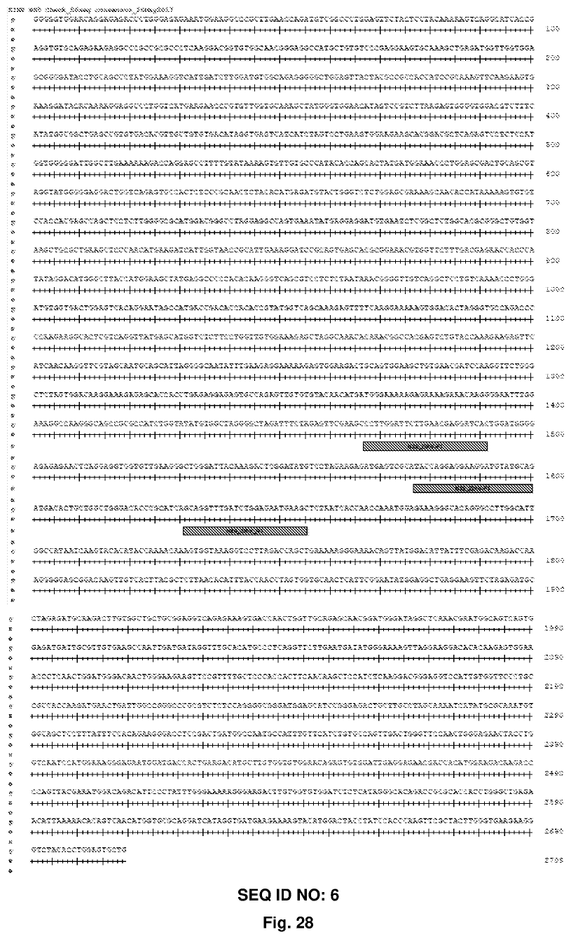

All 4 samples tested positive for DENV2 (FIG. 21A left panel) and there was no cross reactivity in other channels (FAM, TxR) within Mix 2 (FIG. 21A right panel). No cross reactivity was seen in Mix 1 and Mix 1 and 2 IC are stable (FIG. 21B).

### DENV4 Samples

All 4 samples tested positive for DENV4 (FIG. 22A left panel) and there was no cross reactivity in other channels (FAM, Hex) within Mix 2 (FIG. 22A right panel). No cross reactivity was seen in Mix 1 and Mix 1 and 2 IC are stable (FIG. 22B).

In the present disclosure, one example of an optimised multiplex real-time TaqMan-based RT-PCR assay capable of differentially detect six different targets (CHIKV, 4 serotypes of DENV (i.e. DENV1, DENV2, DENV3 and DENV4), and ZIKV) in the whole blood of patients was developed.

Based on the LoD values and the validation experiments with patient samples, the multiplex assay described herein has been shown to be a sensitive and specific assay that is able to successfully differentiate the detection of the six viral targets.

Table 4 below shows the optimised multiplex real-time TaqMan-based RT-PCR for six different targets—CHIKV, 4 serotypes of DENV (DENV1, DENV2, DENV3 and DENV4) and ZIKV, with a clear result interpretation and reporting algorithm.

TABLE 4
Multiplex Assay Interpretation and Reporting Algorithm

| Mix 1 | | | Mix 2 | | | IC | Data Analysis and conclusion | | |
|---|---|---|---|---|---|---|---|---|---|
| ZIKV | DENV1 | DENV3 | CHIKV | DENV2 | DENV4 | β-actin | Interpretation | Reporting | Actions |
| — | — | — | — | — | — | — | Inconclusive | Specimen | Repeat |
| | | | | | | | | inconclusive | extraction |
| | | | | | | | | for presence | and |
| | | | | | | | | of Zika, | RT-PCR |
| | | | | | | | | dengue, and | |
| | | | | | | | | chikungunya | |
| — | — | — | — | — | — | + | Negative | No Zika, | No |
| | | | | | | | | dengue, or | further |
| | | | | | | | | chikungunya | testing |
| | | | | | | | | RNA detected | required |
| | | | | | | | | by RT-PCR | |
| + | — | — | — | — | — | +/− | Positive for | Zika RNA | ZIKV |
| | | | | | | | ZIKV, but | detected | single-plex |
| | | | | | | | negative for | by RT-PCR. | can be run |
| | | | | | | | CHIKV and | | for |
| | | | | | | | DENV. | | confirmation |
| | | | | | | | | | (2_{nd} tier |
| | | | | | | | | | testing) |
| — | + | — | — | — | — | +/− | Positive for | Dengue 1 | DENV1 |
| | | | | | | | DENV1, but | RNA detected | single-plex |
| | | | | | | | negative | by RT-PCR. | can be run |
| | | | | | | | for ZIKV, | | for |
| | | | | | | | DENV2, | | confirmation |
| | | | | | | | DENV3, | | (2_{nd} tier |
| | | | | | | | DENV4 | | testing) |
| | | | | | | | and CHIKV. | | |
| — | — | + | — | — | — | +/− | Positive for | Dengue 3 | DENV3 |
| | | | | | | | DENV3, but | RNA | single-plex |
| | | | | | | | negative | detected | can be run |
| | | | | | | | for ZIKV, | by RT-PCR. | for |
| | | | | | | | DENV1, | | confirmation |
| | | | | | | | DENV2, | | (2nd tier |
| | | | | | | | DENV4 | | testing) |
| | | | | | | | and CHIKV. | | |
| — | — | — | + | — | — | +/− | Positive for | Chikungunya | CHIKV |
| | | | | | | | CHIKV, but | RNA | single-plex |
| | | | | | | | negative | detected | can be run |
| | | | | | | | for ZIKV | by RT-PCR. | for |
| | | | | | | | and DENV. | | confirmation |
| | | | | | | | | | (2nd tier |
| | | | | | | | | | testing) |
| — | — | — | — | + | — | +/− | Positive for | Dengue 2 | DENV2 |
| | | | | | | | DENV2, but | RNA | single-plex |
| | | | | | | | negative | detected | can be run |
| | | | | | | | for ZIKV, | by RT-PCR. | for |
| | | | | | | | DENV1, | | confirmation |
| | | | | | | | DENV3, | | (2nd tier |
| | | | | | | | DENV4 and | | testing) |
| | | | | | | | CHIKV. | | |
| — | — | — | — | — | + | +/− | Positive for | Dengue 4 | DENV4 |
| | | | | | | | DENV4, but | RNA | single-plex |
| | | | | | | | negative | detected | can be run |
| | | | | | | | for ZIKV, | by RT-PCR. | for |
| | | | | | | | DENV1, | | confirmation |
| | | | | | | | DENV2, | | (2nd tier |
| | | | | | | | DENV3 | | testing) |
| | | | | | | | and CHIKV. | | |

TABLE 5
Chikungunya strains used for E1 glycoprotein consensus sequence

| | | | | Collection | |
|---|---|---|---|---|---|
| Accession | Name | Size | Year | Date | Country |
| EF027136 | IND-06-MH2 | 11,800 | 2006 | 2005-2006 | India |
| EF452493 | AF15561 | 12,036 | 2007 | 2007 | Thailand |
| EF452494 | TSI-GSD-218-VR1 | 12,036 | 2007 | 2007 | USA |
| EU244823 | ITA07-RA1 | 11,788 | 2007 | 2007 | Italy |
| EU703759 | MY002IMR/06/BP | 12,028 | 2006 | 2006 | Malaysia |
| FJ445426 | isolate LKEHCH13908 | 11,717 | 2008 | Apr. 2008 | Sri Lanka |
| FJ445430 | isolate SGEHICHD93508 | 11,722 | 2008 | Jul. 2008 | Singapore |
| FJ445433 | isolate SGEHICHS422808 | 11,729 | 2008 | Aug. 2008 | Singapore |
| FJ513629 | isolate LK(PB)CH1608 | 11,716 | 2008 | Mar. 2008 | Sri Lanka |
| FJ807896 | 0611aTw | 11,811 | 2006 | 2006 | Singapore |
| FJ807898 | 0810aTw | 11811 | 2008 | 2008 | Bangladesh |
| FJ807899 | 0810bTw | 11811 | 2008 | 2008 | Malaysia |
| FJ959103 | BNI-CHIKV 899 | 11,832 | 2006 | 2006 | Mauritius |
| FR717336 | complete genome, isolate | 11,559 | 2005 | Dec. 26, 2005 | France |
| | IMTSSA6424S | | | | |
| GU301779 | isolate CU-Chik009 | 11,811 | 2009 | Sep. 04, 2009 | Thailand |
| HM045804 | IPD/A SH 2807 | 11,847 | 2010 | 2010 | Senegal |
| HM045817 | HD 180760 | 11,832 | 2005 | Nov. 2005 | Senegal |
| HQ456251 | Com25 | 11,836 | 2004- | 2004-2005 | Indian ocean |
| | | | 2005 | | |
| HQ456252 | strain COMJ | 11,836 | 2004- | 2004-2005 | Indian ocean |
| | | | 2005 | | |
| HQ456255 | Lamu33 | 11,836 | 2004- | 2004-2005 | Indian ocean |
| | | | 2005 | | |
| JX088705 | GD05/2010 | 11,811 | 2010 | 2010 | China |
| KC614648 | isolate Yem-11, complete | 11,778 | 2011 | Jan. 25, 2011 | Yemen |
| | sequence | | | | |
| KC862329 | isolate NL10/152 | 11,836 | 2010 | 2010 | Indonesia |
| KF318729 | isolate chik-sy | 12,017 | 2012 | Jul. 06, 2012 | China |
| KF590564 | 10Mdy7 | 11,695 | 2010 | 2010 | Myanmar |
| KJ679577 | isolate CHIKV STMWG01 | 11,567 | 2011 | Sep. 12, 2011 | India |
| KJ689452 | Yap 13-2039 | 12,042 | 2013 | Nov. 2013 | Micronesia |
| KJ796844 | RGCB730/09 | 11,764 | 2009 | Aug. 04, 2009 | India |
| KM673291 | isolate DH130003 | 11,979 | 2013 | Jan. 2013 | Indonesia |
| KM923917 | M125 | 11,837 | 2007 | Mar. 09, 2007 | Malaysia |
| KP003808 | MADOPY1 | 11,562 | 2006 | 2006 | Madagascar |
| KP003809 | OPY4 | 11,791 | 2006 | 2006 | Mayotte |
| KP003812 | GABOPY1 | 11,561 | 2007 | 2007 | Gabon |
| KP003813 | BRAZZA_MRS1 | 11,726 | 2011 | 2011 | Republic of |
| | | | | | the Congo |
| KP164567 | isolate AMA2798/H804298 | 11,715 | 2014 | Aug. 28, 2014 | Brazil |
| KP164568 | isolate BHI3734/H804698 | 11,812 | 2014 | Aug. 26, 2014 | Brazil |
| KP164571 | isolate PER160/H803609 | 12,189 | 2014 | Jul. 03, 2014 | Brazil |
| KP164572 | isolate TR206/H804187 | 12,053 | 2014 | Aug. 21, 2014 | Brazil |
| KP851709 | isolate InDRE 51CHIK | 11,989 | 2014 | Oct. 15, 2014 | Mexico |
| KR046227 | isolate VE53_20 | 12,003 | 2014 | Aug. 30, 2014 | Trinidad and |
| | | | | | Tobago |
| KR264949 | isolate PR-S4 | 12,014 | 2014 | Jul. 15, 2014 | Puerto Rico |
| KR559470 | WHCHK1 | 12,004 | 2014 | Nov. 2014 | Puerto Rico |
| KT192707 | 11540 | 11,889 | 2014 | Oct. 31, 2014 | Nicaragua |
| KT308159 | isolate CPCC007800Y01 | 11,631 | 2012 | 2012 | Philippines |
| KT327163 | isolate CH0008 | 12,193 | 2014 | 2014 | Mexico |
| KT449801 | LR2006_OPY1 | 11,796 | 2006 | 2006 | Reunion |
| KX009167 | isolate Chik435 | 11,765 | 2013 | 2013 | Thailand |
| KX168429 | isolate MUM001-2009-Selangor | 11,900 | 2008 | 2009 | Malaysia |
| KX262987 | CHIKV/Homo sapiens/THA/SVO- | 11,671 | 1996 | 1996 | Thailand |
| | 451-96/1996 | | | | |
| KX262991 | CHIKV/Homo sapiens/SXM/H- | 11,950 | 2003 | 2003 | Saint Martin |
| | 20235-STMARTIN-2013/2003 | | | | |
| KX262992 | CHIKV/Homo sapiens/GLP/YO- | 11,294 | 2014 | Jan. 05, 2014 | Guadeloupe |
| | 111213/2014 | | | | |
| KX262994 | CHIKV/Homo sapiens/GUF/YO- | 11,702 | 2014 | Jan. 21, 2014 | French |
| | 123223/2014 | | | | Guiana |
| KX262996 | CHIKV/Homo | 11,570 | 2006 | 2006 | Cameroon |
| | sapiens/CMR/667/2006 | | | | |
| KX262997 | CHIKV/Homo sapiens/MYS/BS- | 11,669 | 2009 | 2009 | Malaysia |
| | 285-C2/2009 | | | | |
| KX496989 | Homo sapiens/COL/UF-1/2016 | 12,037 | 2016 | Feb. 09, 2016 | Colombia |
| KY575565 | CHIKV/Homo | 11,643 | 2014 | 2014 | USA |
| | sapiens/USA/IDR1400024561/2014 | | | | |
| KY575567 | CHIKV/Homo | 11,758 | 2006 | 2006 | USA |
| | sapiens/USA/91077/2006 | | | | |
| KY703888 | CHIKV/Homo | 11,456 | 2015 | 2015 Aug. 15 | Nicaragua: |
| | sapiens/NIC/1885.1D/2015 | | | | Managua |
| KY751908 | isolate IN16C1 | 11,796 | 2016 | 2016 | Australia: |
| | | | | | imported |
| | | | | | from India |
| LN898093 | Caribbean strain, isolate M100 | 12,230 | 2013 | Dec. 2013 | Martinique |
| | IND91 | 11737 | | | India |
| | SGP11 | 11708 | | | Singapore |
| | SGP7 | 11738 | | | Singapore |
| | CNR20235 | 11917 | | | Caribbean |
| | | | | | islands |

TABLE 6
DENV1 strains used for NS5 consensus sequence

| | | Subtype/ | | |
|---|---|---|---|---|
| Accession | Strain | Genotype | Year | Country |
| AY620951 | My00D136393 | I | 2000 | Myanmar |
| AY732464 | ThD1_K0407 01 | I | 2001 | Thailand |
| AM746216 | 6633 | I | 2004 | Saudi Arabia |
| AY835999 | ZJ01/2004 | I | 2004 | China |
| FR666922 | D1/Malaysia/33087/04 | I | 2004 | Malaysia |
| EU081226 | D1/SG/05K814DK1/2005 | I | 2005 | Singapore |
| FJ196844 | GD02/06 | I | 2006 | China |
| JN415533 | Vietnam 2006 | I | 2006 | Vietnam |
| AB608787 | SDDF1543 | I | 2008 | Taiwan |
| GU131792 | DENV-1/VN/BID-V4034/2008 | I | 2008 | Vietnam South |
| JN415521 | Singapore 2008 | I | 2008 | Singapore |
| JN415534 | Vietnam 2008a | I | 2008 | Vietnam |
| KC172829 | XB998_Laos_2008 | I | 2008 | Laos |
| KR919821 | TSV08 | I | 2008 | Australia |
| GU131895 | DENV-1/IPC/BID- | I | 2009 | Cambodia |
| | V3787/2009 | | | |
| HQ891316 | DV1_SL_2009d | I | 2009 | Sri Lanka |
| KC182084 | LNT1975_Laos_2010 | I | 2010 | Laos |
| KC848576 | SO/DB118/2011 | I | 2011 | Somalia |
| KJ649286 | DENV-1-Jeddah | I | 2011 | Saudi Arabia |
| KR919805 | Bali 2011 | I | 2011 | Indonesia |
| KJ726662 | SL_2012_GS0319 | I | 2012 | Sri Lanka |
| KR919808 | Cairns 2012 | I | 2012 | Australia |
| KF184975 | Angola_2013 | I | 2013 | Angola |
| KR919817 | Van 2013 | I | 2013 | Vanuatu |
| KR919816 | ET2014 | I | 2014 | East Timor |
| LC002828 | D1/Hu/Saitama/NIID100/2014 | I | 2014 | Japan |
| FN825674 | D1/Malaysia/36046/05 | III | 2005 | Malaysia |
| U88535 | Nauru Island, Western Pacific | IV | 1974 | Nauru |
| FJ196846 | GD95/95 | IV | 1995 | China |
| JN415499 | ET00 243 | IV | 2000 | East Timor |
| JN415515 | Palau 2000 | IV | 2000 | Palau |
| AY630407 | FP/01/192206 | IV | 2001 | French Polynesia |
| DQ672564 | HawO3663 | IV | 2001 | Hawaii |
| EU863650 | CHI3336-02 | IV | 2002 | Chile: Easter Island |
| JN415503 | Fiji 2002 | IV | 2002 | Fiji |
| KF559254 | M49440 | IV | 2002 | Myanmar |
| AB195673 | NIID03-41 | IV | 2003 | Seychelles |
| FJ196842 | GD66/03 | IV | 2003 | China |
| AB204803 | NIID04-27 | IV | 2004 | Federated States of |
| | | | | Micronesia (Yap) |
| JN415513 | Malaysia 2010 | IV | 2010 | Malaysia |
| JQ915080 | NC10/080810-1138 | IV | 2010 | New Caledonia |
| JX298570 | Fiji 2011b | IV | 2011 | Fiji |
| KR919815 | PNG 2011 | IV | 2011 | Papua New Guinea |
| KR919818 | PNG 2014b | IV | 2014 | Papua New Guinea |
| KR919810 | Tully 2015 | IV | 2015 | Australia |
| JQ922544 | IND/631288/1963 | V | 1963 | India |
| AF425625 | IBH 28328 | V | 1968 | Nigeria |
| GU131962 | DENV-1/MX/BID-V3669/2007 | V | 2007 | Mexico |
| GQ357692 | SG(EHI)DED65008 | V | 2008 | Singapore |
| GU131863 | DENV-1/BR/BID-V3490/2008 | V | 2008 | Brazil |
| JN415506 | Guyana 2008 | V | 2008 | Guyana |
| JN903579 | D1/IN/RGCB419/2008 | V | 2008 | India |
| KC692512 | HNRG25001 | V | 2010 | Argentina |
| KJ189367 | DENV-1/PR/BID-V8188/2010 | V | 2010 | Puerto Rico |
| KF864667 | Zj/yw01 | V | 2013 | China |
| KM458188 | US/DB167/2014 | V | 2014 | USA |
| AF309641 | D1/H/IMTSSA/98/658 | I | 1998 | Cambodia |
| AY732483 | ThD1_0008_81 | I | 1981 | Thailand |
| EU081262 | D1/SG/05K4173DK1/2005 | I | 2005 | Singapore |
| KU365900 | D1/Taiwan/806KH1405a | I | 2014 | Taiwan |
| KX225493 | GZ-12/M/GZ/2014/DEV1 | I | 2015 | China |
| KP406802 | DenKor-02 | I | 2005 | South Korea/travelled to |
| | | | | Indonesia |
| KF955444 | KH/BID-V3784/2007 | I | 2007 | Cambodia |
| DQ672556 | FP0203 | IV | 2001 | French Polynesia |
| DQ672560 | HawM2516 | IV | 2001 | Hawaii/French Polynesia |
| EU179861 | DS212-110306 | IV | 2006 | Brunei |
| KP406803 | DenKor-07 | IV | 2007 | South Korea/travelled to |
| | | | | Philippines |
| JQ675358 | DENV-1/BOL-KW010 | V | 2010 | USA |
| HQ332182 | VE_61006_2006 | V | 2006 | Venezuela |
| AY762084 | Singapore 8114/93 | V | 1993 | Singapore |
| AF311957 | BR/97-409 | V | 1997 | Brazil |
| AF513110 | BR/01-MR | V | 2001 | Brazil |
| GU131949 | CO/BID-V3383/2006 | V | 2006 | Colombia: Norte de |
| | | | | Santander |
| KJ189368 | MX/BID-V8195/2012 | V | 2012 | Mexico |
| KJ189366 | PR/BID-V8187/2010 | V | 2012 | Puerto Rico |

TABLE 7
DENV2 strains used for NS5 consensus sequence

| | | Subtype/ | | | |
|---|---|---|---|---|---|
| Accession | Strain name | Genotype | Lineage | Year | Country |
| EU482449 | DENV-2/VN/BID- | AA | AA1 | 2006 | Vietnam |
| | V1004/2006 | | | | |
| FM210213 | MD1504 | AA | AA1 | 2005 | Vietnam |
| FM210202 | DF768 | AA | AA1 | 2004 | Vietnam |
| FJ639702 | DENV-2/KH/BID- | AA | AA1 | 2003 | Cambodia |
| | V2030/2003 | | | | |
| EU482780 | DENV-2/VN/BID- | AA | AA1 | 2003 | Vietnam |
| | V758/2003 | | | | |
| FJ639697 | DENV-2/KH/BID- | AA | AA1 | 2001 | Cambodia |
| | V2020/2001 | | | | |
| EU596489 | DENV-2/US/BID- | AA | AA2 | 2007 | USA |
| | V1411/2007 | | | | |
| EU482544 | DENV-2/US/BID- | AA | AA2 | 2006 | USA |
| | V1031/2006 | | | | |
| EU482556 | DENV-2/US/BID- | AA | AA2 | 2005 | USA |
| | V1045/2005 | | | | |
| EU687214 | DENV-2/US/BID- | AA | AA2 | 2004 | USA |
| | V1435/2004 | | | | |
| EU687240 | DENV-2/US/BID- | AA | AA2 | 2003 | USA |
| | V1492/2003 | | | | |
| EU482722 | DENV-2/US/BID- | AA | AA2 | 2002 | USA |
| | V591/2002 | | | | |
| EU482593 | DENV-2/US/BID- | AA | AA2 | 2001 | USA |
| | V854/2001 | | | | |
| GQ398270 | DENV-2/PR/2DN/1994 | AA | AA2 | 1994 | Puerto Rico |
| FJ898450 | DENV-2/VI/BID- | AA | AA2 | 1990 | Virgin Islands |
| | V2948/1990 | | | | |
| FJ850072 | DENV-2/BR/BID- | AA | AA3 | 2000 | Brazil |
| | V2376/2000 | | | | |
| GQ868552 | DENV-2/CO/BID- | AA | AA3 | 1998 | Colombia |
| | V3368/1998 | | | | |
| FJ898465 | DENV-2/VE/BID- | AA | AA3 | 1998 | Venezuela |
| | V2941/1998 | | | | |
| FJ850106 | DENV-2/VE/BID- | AA | AA4 | 2008 | Venezuela |
| | V2476/2008 | | | | |
| GQ868558 | DENV-2/CO/BID- | AA | AA4 | 2007 | Colombia |
| | V3375/2007 | | | | |
| EU482604 | DENV-2/VE/BID- | AA | AA4 | 2007 | Venezuela |
| | V1095/2007 | | | | |
| FJ850088 | DENV-2/BR/BID- | AA | AA4 | 2006 | Brazil |
| | V2396/2006 | | | | |
| HQ332184 | VE_61069_2006 | AA | AA4 | 2006 | Venezuela |
| FJ850085 | DENV-2/BR/BID- | AA | AA4 | 2005 | Brazil |
| | V2393/2005 | | | | |
| FJ024473 | DENV-2/CO/BID- | AA | AA4 | 2005 | Colombia |
| | V1594/2005 | | | | |
| FJ850082 | DENV-2/BR/BID- | AA | AA4 | 2004 | Brazil |
| | V2390/2004 | | | | |
| FJ639734 | DENV-2/VE/BID- | AA | AA4 | 2003 | Venezuela |
| | V2160/2003 | | | | |
| FJ850076 | DENV-2/BR/BID- | AA | AA4 | 2002 | Brazil |
| | V2382/2002 | | | | |
| JN819408 | DENV-2/VE/BID- | AA | AA4 | 2001 | Venezuela |
| | V2161/2001 | | | | |
| GQ199892 | DENV-2/JM/BID- | AA | AA5 | 2007 | Jamaica |
| | V2963/2007 | | | | |
| EU596491 | DENV-2/US/BID- | AA | AA5 | 2007 | USA |
| | V1413/2007 | | | | |
| FJ898453 | DENV-2/VI/BID- | AA | AA5 | 2005 | Virgin Islands |
| | V2960/2005 | | | | |
| FJ898451 | DENV-2/DO/BID- | AA | AA5 | 2003 | Dominican |
| | V2955/2003 | | | | Republic |
| FJ898460 | DENV-2/KN/BID- | AA | AA5 | 2001 | Saint Kitts and |
| | V2951/2001 | | | | Nevis |
| AF208496 | DEN2/H/IMTSSA- | AA | AA5 | 1998 | Martinique |
| | MART/98-703 | | | | |
| AY702037 | Cuba89/97 | AA | AA5 | 1997 | Cuba |
| HQ705624 | DENV-2/NI/BID- | AA | AA6 | 2009 | Nicaragua |
| | V4914/2009 | | | | |
| FJ898439 | DENV-2/MX/BID- | AA | AA6 | 2008 | Mexico |
| | V2964/2008 | | | | |
| GQ868515 | DENV-2/MX/BID- | AA | AA6 | 2007 | Mexico |
| | V3713/2007 | | | | |
| GQ868497 | DENV-2/MX/BID- | AA | AA6 | 2006 | Mexico |
| | V3654/2006 | | | | |
| FJ850067 | DENV-2/NI/BID- | AA | AA6 | 2006 | Nicaragua |
| | V2331/2006 | | | | |
| GQ199894 | DENV-2/MX/BID- | AA | AA6 | 2005 | Mexico |
| | V2959/2005 | | | | |
| FJ898461 | DENV-2/BZ/BID- | AA | AA6 | 2002 | Belize |
| | V2952/2002 | | | | |
| GU369819 | CAM7786 | AA | AA6 | 2002 | Mexico |
| JF730051 | DENV-2/NI/BID- | AA | AA7 | 2009 | Nicaragua |
| | V5072/2009 | | | | |
| FJ639829 | DENV-2/TH/BID- | Al | AI-1 | 2001 | Thailand |
| | V2154/2001 | | | | |
| FJ639718 | DENV-2/KH/BID- | Al | AI-2 | 2008 | Cambodia |
| | V2068/2008 | | | | |
| HQ541799 | DENV-2/US/BID- | All | All | 2010 | USA |
| | V4825/2010 | | | | |
| FJ906959 | DENV-2/PG/BID- | All | All | 2008 | Papua New |
| | V2618/2008 | | | | Guinea |
| HQ891023 | DENV-2/TW/BID- | All | All | 2008 | Taiwan |
| | V5054/2008 | | | | |
| EU056811 | IQT-1950 | AM | AM | 1995 | Peru |
| GQ868590 | DENV-2/MX/BID- | AM | AM | 1992 | Mexico |
| | V3356/1992 | | | | |
| JQ922551 | DENV- | C | C1 | 2005 | India |
| | 2/IND/053598/2005 | | | | |
| GQ252676 | DENV-2/LK/BID- | C | C1 | 2003 | Sri Lanka |
| | V2421/2003 | | | | |
| DQ448231 | GWL18 INDI-01 | C | C1 | 2001 | India |
| GQ398265 | DENV- | C | C2 | 2008 | Singapore |
| | 2/SG/07K3608DK1/2008 | | | | |
| EU179859 | DS09-280106 | C | C2 | 2006 | Brunei |
| EU482672 | DENV-2/VN/BID- | C | C2 | 2006 | Viet Nam |
| | V735/2006 | | | | |
| FJ196853 | GD01/03 | C | C2 | 2003 | China |
| DQ645545 | 1183-DF-06/17/2002 | C | C2 | 2002 | Taiwan |
| AB189122 | strain 98900663 DHF DV- | C | C2 | 1998 | Indonesia |
| | 2 | | | | |

TABLE 8
DENV3 strains used for NS5 consensus sequence

| | | Subtype/ | Sequence | | Collection | |
|---|---|---|---|---|---|---|
| Accession | Strain Name | Genotype | Length | Year | Date | Country |
| AB189127 | 98901517 DHF DV-3 | 3-I | 10707 | -N/A- | -N/A- | Indonesia |
| AB189126 | 98901437 DSS DV-3 | 3-I | 10707 | -N/A- | -N/A- | Indonesia |
| KC762685 | MKS-0388 | 3-I | 10707 | 2008 | Feb. 15, 2008 | Indonesia |
| KC762681 | MKS-0057 | 3-I | 10707 | 2007 | Jun. 22, 2007 | Indonesia |
| JQ920479 | PF96/150296-46183 | 3-I | 10671 | 1996 | Feb. 15, 1996 | French Polynesia |
| JQ920485 | NC96/211096-4631 | 3-I | 10663 | 1996 | Oct. 21, 1996 | New Caledonia |
| FJ898456 | DENV-3/WS/BID-V2973/1995 | 3-I | 10663 | 1995 | 1995 | Samoa |
| JQ920488 | WF95/050495-1650 | 3-I | 10671 | 1995 | Apr. 05, 1995 | Wallis and Futuna |
| AY744685 | PF94/136116 | 3-I | 10707 | 1994 | 1994 | French Polynesia |
| FJ898455 | DENV-3/CK/BID-V2972/1991 | 3-I | 10663 | 1991 | 1991 | Cook Islands |
| KJ622199 | HN/2013/110 | 3-II | 10710 | 2013 | 2013 | China |
| KF824903 | YN02 | 3-II | 10707 | 2013 | 2013 | China |
| GU189648 | DTID-ZJU04 | 3-II | 10173 | 2009 | 2009 | China |
| GU131946 | DENV-3/IPC/BID-V4314/2008 | 3-II | 10631 | 2008 | 2008 | Cambodia |
| FJ461337 | DENV-3/VN/BID-V1946/2008 | 3-II | 10623 | 2008 | 2008 | Viet Nam |
| FJ432741 | DENV-3/VN/BID-V1810/2007 | 3-II | 10638 | 2007 | 2007 | Viet Nam |
| GU131908 | DENV-3/IPC/BID-V3820/2006 | 3-II | 10511 | 2006 | 2006 | Cambodia |
| EU482457 | DENV-3/VN/BID-V1013/2006 | 3-II | 10663 | 2006 | 2006 | Viet Nam |
| GQ868629 | DENV-3/KH/BID-V2087/2005 | 3-II | 10660 | 2005 | 2005 | Cambodia |
| FJ639726 | DENV-3/KH/BID-V2083/2004 | 3-II | 10648 | 2004 | 2004 | Cambodia |
| FJ639725 | DENV-3/KH/BID-V2082/2003 | 3-II | 10648 | 2003 | 2003 | Cambodia |
| FJ639721 | DENV-3/KH/BID-V2078/2002 | 3-II | 10648 | 2002 | 2002 | Cambodia |
| GQ868626 | DENV-3/KH/BID-V2075/2001 | 3-II | 10663 | 2001 | 2001 | Cambodia |
| FJ744728 | DENV-3/TH/BID-V2314/2001 | 3-II | 10664 | 2001 | 2001 | Thailand |
| KJ737429 | C0360/94 | 3-II | 10707 | 1994 | 1994 | Thailand |
| KF955449 | DENV-3/VE/BID-V1121/36892.5 | 3-III | 10421 | -N/A- | -N/A- | Venezuela |
| KF973478 | DENV-3/NI/BID-V7646/2012 | 3-III | 10584 | 2012 | 2012 | Nicaragua |
| KF973483 | DENV-3/NI/BID-V7665/2011 | 3-III | 10509 | 2011 | 2011 | Nicaragua |
| JF937638 | DENV-3/NI/BID-V5517/2010 | 3-III | 10681 | 2010 | 2010 | Nicaragua |
| JF808120 | D3BR/AL95/2009 | 3-III | 10707 | 2009 | 2009 | Brazil |
| KF971709 | DENV-3/NI/BID-V5453/2009 | 3-III | 10678 | 2009 | 2009 | Nicaragua |
| KJ189292 | DENV-3/PE/BID-V7081/2009 | 3-III | 10696 | 2009 | 2009 | Peru |
| FJ850094 | DENV-3/BR/BID-V2403/2008 | 3-III | 10662 | 2008 | 2008 | Brazil |
| KJ189298 | DENV-3/PE/BID-V7087/2008 | 3-III | 10692 | 2008 | 2008 | Peru |
| FJ639826 | DENV-3/VE/BID-V2267/2008 | 3-III | 10654 | 2008 | 2008 | Venezuela |
| KU509278 | DENV3-254 | 3-III | 10270 | 2007 | 2007 | Barbados |
| FJ850092 | DENV-3/BR/BID-V2400/2007 | 3-III | 10648 | 2007 | 2007 | Brazil |
| EU596492 | DENV-3/US/BID-V1415/2007 | 3-III | 10648 | 2007 | 2007 | USA |
| EU529683 | DENV-3/VE/BID-V1102/2007 | 3-III | 10649 | 2007 | 2007 | Venezuela |
| GU131849 | DENV-3/BR/BID-V3430/2006 | 3-III | 10525 | 2006 | 2006 | Brazil |
| JX669496 | 603/BR-PE/06 | 3-III | 10709 | 2006 | 2006 | Brazil |
| GU131954 | DENV-3/CO/BID-V3404/2006 | 3-III | 10525 | 2006 | 2006 | Colombia |
| FJ898441 | DENV-3/MX/BID-V2987/2006 | 3-III | 10663 | 2006 | 2006 | Mexico |
| KJ189295 | DENV-3/PE/BID-V7084/2006 | 3-III | 10694 | 2006 | 2006 | Peru |
| KF955456 | DENV-3/PR/BID-V1728/2006 | 3-III | 10645 | 2006 | 2006 | Puerto Rico |
| EU482555 | DENV-3/US/BID-V1043/2006 | 3-III | 10648 | 2006 | 2006 | USA |
| HQ332171 | VE_61035_2006 | 3-III | 10707 | 2006 | 2006 | Venezuela |
| JX669500 | 249/BR-PE/05 | 3-III | 10707 | 2005 | 2005 | Brazil |
| FJ898444 | DENV-3/CO/BID-V2986/2005 | 3-III | 10663 | 2005 | 2005 | Colombia |
| KJ189299 | DENV-3/PE/BID-V7088/2005 | 3-III | 10692 | 2005 | 2005 | Peru |
| FJ182008 | DENV-3/US/BID-V1618/2005 | 3-III | 10648 | 2005 | 2005 | USA |
| JX669495 | 145/BR-PE/04 | 3-III | 10707 | 2004 | 2004 | Brazil |
| GQ868575 | DENV-3/CO/BID-V3400/2004 | 3-III | 10663 | 2004 | 2004 | Colombia |
| FJ182006 | DENV-3/US/BID-V1616/2004 | 3-III | 10648 | 2004 | 2004 | USA |
| FJ639791 | DENV-3/VE/BID-V2224/2004 | 3-III | 10647 | 2004 | 2004 | Venezuela |
| EF643017 | D3BR/RP1/2003 | 3-III | 10707 | 2003 | 2003 | Brazil |
| FJ898440 | DENV-3/MX/BID-V2985/2003 | 3-III | 10663 | 2003 | 2003 | Mexico |
| JF808129 | D3PY/AS10/03 | 3-III | 10707 | 2003 | 2003 | Paraguay |
| KT726348 | Cuba_21_2002 | 3-III | 10663 | 2002 | 2002 | Cuba |
| KF955505 | DENV-3/GD/BID-V3930/2002 | 3-III | 10653 | 2002 | 2002 | Grenada |
| JF808123 | D3PY/AS12/02 | 3-III | 10707 | 2002 | 2002 | Paraguay |
| FJ898459 | DENV-3/TT/BID-V2982/2002 | 3-III | 10663 | 2002 | 2002 | Trinidad and Tobago |
| FJ547083 | DENV-3/US/BID-V2119/2002 | 3-III | 10649 | 2002 | 2002 | USA |
| FJ898462 | DENV-3/AI/BID-V2976/2001 | 3-III | 10663 | 2001 | 2001 | Anguilla |
| KT726342 | Cuba_553_2001 | 3-III | 10663 | 2001 | 2001 | Cuba |
| KF955468 | DENV-3/PR/BID-V2116/2001 | 3-III | 10640 | 2001 | 2001 | Puerto Rico |
| GQ868616 | DENV-3/LC/BID-V3929/2001 | 3-III | 10663 | 2001 | 2001 | Saint Lucia |
| FJ898457 | DENV-3/EC/BID-V2975/2000 | 3-III | 10663 | 2000 | 2000 | Ecuador |
| KF955466 | DENV-3/PR/BID-V2102/2000 | 3-III | 10596 | 2000 | 2000 | Puerto Rico |
| KF955465 | DENV-3/PR/BID-V2101/2000 | 3-III | 10614 | 2000 | 2000 | Puerto Rico |
| GQ199886 | DENV-3/NI/BID-V2419/1998 | 3-III | 10646 | 1998 | 1998 | Nicaragua |
| FJ882575 | DENV-3/MZ/BID-V2418/1985 | 3-III | 10663 | 1985 | 1985 | Mozambique |
| EU367962 | 07CHLS001 | -N/A- | 10707 | -N/A- | -N/A- | China |
| AY858046 | PI64 | -N/A- | 10707 | -N/A- | -N/A- | Indonesia |
| KJ830751 | Jeddah-2014 | -N/A- | 10635 | 2014 | Jan. 26, 2014 | Saudi Arabia |
| KF954946 | 13GDZDVS30B | -N/A- | 10686 | 2013 | Aug. 08, 2013 | China |
| KU216208 | Rajasthan.India/DMRC/ | -N/A- | 10672 | 2013 | Nov. 11, 2013 | India |
| | Balotra87/2013 | | | | | |
| KX380842 | D3/SG/CT37/2013 | -N/A- | 10676 | 2013 | 2013 | Singapore |
| KC261634 | GZ/10476/2012 | -N/A- | 10630 | 2012 | 2012 | China |
| KX380839 | D3/SG/CT7/2012 | -N/A- | 10674 | 2012 | 2012 | Singapore |
| KU509286 | DENV3-9468 | -N/A- | 10493 | 2011 | 2011 | India |
| KU509280 | DENV3-1631 | -N/A- | 10261 | 2011 | 2011 | Thailand |
| KC762692 | MKS-WS78 | -N/A- | 10707 | 2010 | Mar. 22, 2010 | Indonesia |
| JF504679 | ZJYW2009 | -N/A- | 10685 | 2009 | September 2009 | China |
| KU509281 | DENV3-2994 | -N/A- | 10259 | 2009 | 2009 | India |
| KF041258 | D3/Pakistan/45251/2009 | -N/A- | 10675 | 2009 | 2009 | Pakistan |
| KU509282 | DENV3-3140 | -N/A- | 10568 | 2009 | 2009 | Senegal |
| GU370052 | SGEHI(D3)0040Y09 | -N/A- | 10250 | 2009 | January 2009 | Singapore |
| FJ639716 | DENV-3/KH/BID-V2054/2008 | -N/A- | 10648 | 2008 | 2008 | Cambodia |
| GQ466079 | DEL-72 | -N/A- | 10680 | 2008 | 2008 | India |
| KF041254 | D3/Pakistan/56/2008 | -N/A- | 10675 | 2008 | 2008 | Pakistan |
| FJ644564 | ND143 | -N/A- | 10707 | 2007 | 2007 | India |
| KF041255 | D3/Pakistan/55505/2007 | -N/A- | 10675 | 2007 | 2007 | Pakistan |
| KF041259 | D3/Pakistan/43298/2006 | -N/A- | 10675 | 2006 | 2006 | Pakistan |
| KU509283 | DENV3-3404 | -N/A- | 10336 | 2006 | 2006 | Sri Lanka |
| AB214880 | D3/Hu/TL029NIID/2005 | -N/A- | 10707 | 2005 | 2005 | East Timor |

TABLE 9
DENV4 strains used for capsid consensus sequence

| | | Subtype/ | Sequence | | Collection | |
|---|---|---|---|---|---|---|
| Accession | Strain Name | Genotype* | Length | Year | Date | Country |
| JQ513345 | H781363 | 4-I | 10604 | 2011 | Mar. 18, 2011 | Brazil |
| KR922405 | 11/1666 | 4-I | 10164 | 2011 | 2011 | Thailand |
| JN638570 | DF patient | 4-I | 10650 | 2008 | 2008 | Cambodia |
| JN638572 | DSS patient | 4-I | 10656 | 2008 | 2008 | Cambodia |
| JN638571 | DHF patient | 4-I | 10656 | 2007 | 2007 | Cambodia |
| KF955510 | DENV-4/KH/BID-V2055/2002 | 4-I | 10586 | 2002 | 2002 | Cambodia |
| KJ160504 | rDENV4 | 4-II | 10650 | NA | -N/A- | Sri Lanka |
| KU523872 | ID-CN27-15 | 4-II | 10653 | 2015 | Apr. 08, 2015 | Indonesia |
| LC069810 | D4/Hu/India/NIID48/2009 | 4-II | 10623 | 2015 | 2015 | Japan |
| KP140942 | MRS 6169642904/2014 | 4-II | 10565 | 2014 | Sep. 11, 2014 | Haiti |
| KT276273 | Haiti/0324/2014 | 4-II | 10649 | 2014 | Sep. 08, 2014 | Haiti |
| KU523871 | PH-CN08-14 | 4-II | 10589 | 2014 | Feb. 13, 2014 | Philippines |
| KP188565 | BR/SJRP/733/2013 | 4-II | 10355 | 2013 | Jan. 30, 2013 | Brazil |
| KP188566 | BR/SJRP/850/2013 | 4-II | 10426 | 2013 | Feb. 08, 2013 | Brazil |
| KU513441 | LRV13/422 | 4-II | 10650 | 2013 | 2013 | Brazil |
| KJ579243 | DENV-4/MT/BR12_TVP17898/2012 | 4-II | 10649 | 2012 | Mar. 28, 2012 | Brazil |
| KJ596666 | DENV-4/MT/BR53_TVP17939/2012 | 4-II | 10649 | 2012 | Mar. 14, 2012 | Brazil |
| KP188558 | BR/SJRP/505/2012 | 4-II | 10572 | 2012 | Mar. 14, 2012 | Brazil |
| KJ596664 | DENV-4/MT/BR50_TVP18148/2012 | 4-II | 10649 | 2012 | Mar. 12, 2012 | Brazil |
| KP188557 | BR/SJRP/500/2012 | 4-II | 10425 | 2012 | Mar. 09, 2012 | Brazil |
| KJ596672 | DENV-4/MT/BR91_TVP17968/2012 | 4-II | 10649 | 2012 | Feb. 03, 2012 | Brazil |
| KP188560 | BR/SJRP/514/2012 | 4-II | 10576 | 2012 | Apr. 02, 2012 | Brazil |
| KC333651 | GZ/9809/2012 | 4-II | 10574 | 2012 | 2012 | China |
| JQ513344 | H780571 | 4-II | 10604 | 2011 | Jan. 13, 2011 | Brazil |
| KT794007 | BR005AM_2011 | 4-II | 10649 | 2011 | Mar. 07, 2011 | Brazil |
| JQ513341 | H780120 | 4-II | 10604 | 2010 | Nov. 21, 2010 | Brazil |
| JQ513331 | H772852 | 4-II | 10604 | 2010 | Jul. 18, 2010 | Brazil |
| JQ513334 | H775222 | 4-II | 10604 | 2010 | Nov. 10, 2010 | Brazil |
| JN983813 | Br246RR/10 | 4-II | 10649 | 2010 | Sep. 08, 2010 | Brazil |
| JF741967 | GZ1D4 | 4-II | 10631 | 2010 | Sep. 26, 2010 | China |
| JQ915084 | PF10/150610-28 | 4-II | 10573 | 2010 | Jun. 15, 2010 | French Polynesia |
| KU509288 | DENV4-61120 | 4-II | 10500 | 2010 | 2010 | Indonesia |
| JX024757 | EHI310A129CY10 | 4-II | 10653 | 2010 | December 2010 | Singapore |
| JQ915081 | PF09/290109-69 | 4-II | 10572 | 2009 | Jan. 29, 2009 | French Polynesia |
| JQ915083 | PF09/080409-93 | 4-II | 10572 | 2009 | Apr. 08, 2009 | French Polynesia |
| JQ915089 | NC09/230909-14518 | 4-II | 10572 | 2009 | Sep. 23, 2009 | New Caledonia |
| JQ915088 | NC09/020409-9266 | 4-II | 10572 | 2009 | Apr. 02, 2009 | New Caledonia |
| JQ915090 | WF09/010409-0001 | 4-II | 10246 | 2009 | Apr. 01, 2009 | Wallis and Futuna |
| KC762698 | MKS-2007 | 4-II | 10653 | 2008 | Apr. 30, 2008 | Indonesia |
| JQ915085 | NC08/200208-409 | 4-II | 10572 | 2008 | Feb. 20, 2008 | New Caledonia |
| KC762696 | MKS-0252 | 4-II | 10653 | 2007 | Aug. 13, 2007 | Indonesia |
| FJ182016 | DENV-4/VE/BID-V1158/2007 | 4-II | 10606 | 2007 | 2007 | Venezuela |
| HQ332176 | VE_61054_2007 | 4-II | 10649 | 2007 | 2007 | Venezuela |
| FJ882583 | DENV-4/VE/BID-V2492/2007 | 4-II | 10592 | 2007 | 2007 | Venezuela |
| KM190936 | VIROAF8 | 4-II | 10592 | 2006 | May 26, 2006 | Thailand |
| GQ398256 | DENV-4/SG/06K2270DK1/2005 | 4-II | 10653 | 2005 | 2005 | Singapore |
| FJ639764 | DENV-4/VE/BID-V2194/2001 | 4-II | 10566 | 2001 | 2001 | Venezuela |
| FJ850095 | DENV-4/VE/BID-V2176/2000 | 4-II | 10558 | 2000 | 2000 | Venezuela |
| EU854297 | DENV-4/US/BID-V1094/1998 | 4-II | 10606 | 1998 | 1998 | USA |
| FJ024476 | DENV-4/CO/BID-V1600/1997 | 4-II | 10606 | 1997 | 1997 | Colombia |
| GQ199881 | DENV-4/US/BID-V2435/1996 | 4-II | 10590 | 1996 | 1996 | USA |
| FJ810417 | DENV-4/US/BID-V2433/1995 | 4-II | 10606 | 1995 | 1995 | USA |
| JF262781 | INH6412 | 4-II | 10649 | 1995 | 1995 | Venezuela |
| JF262782 | Haiti73 | 4-II | 10649 | 1994 | 1994 | Haiti |
| GQ199878 | DENV-4/US/BID-V2429/1994 | 4-II | 10592 | 1994 | 1994 | USA |
| KR011349 | H241 | -N/A- | 10664 | 1956 | Aug. 28, 1956 | Philippines |

*I: Philippines, Thailand and Sri-Lanka
II: Indonesia, Tahiti, Caribbean Islands (Puerto Rico, Dominica) and Central and South America
III: sylvatic

TABLE 10
ZIKV strains used for NS5 consensus sequence

| | | Subtype/ | Sequence | | Collection | |
|---|---|---|---|---|---|---|
| Accession | Strain Name | Genotype | Length | Year | Date | Country |
| EU545988 | EU545988 FSM | Asian | 10272 | 2007 | 2007 | Micronesia |
| JN860885 | FSS13025 | Asian | | 2010 | 2010 | Cambodia |
| KU955593 | KHM/2010/FSS13025 | Asian | 10807 | 2010 | 2010 | Cambodia |
| KU681082 | PHL/2012/CPC-0740 | Asian | 10807 | 2012 | May 09, 2012 | Philippines |
| KF993678 | PLCal_ZV | Asian | | 2013 | 2013 | Canada |
| KJ776791 | H/PF/2013 | Asian | 10807 | 2013 | Nov. 28, 2013 | French Polynesia |
| KX051560 | SK364/13AS | Asian | 10795 | 2013 | Jul. 09, 2013 | Thailand |
| KJ634273 | CK-ISL 2014 | Asian | | 2014 | 2014 | Cook Islands |
| KX447511 | 1_0015_PF | Asian | 10585 | 2014 | January 2014 | French Polynesia |
| KU509998 | Haiti/1225/2014 | Asian | 10807 | 2014 | Dec. 12, 2014 | Haiti |
| KU681081 | THA/2014/SV0127-14 | Asian | 10807 | 2014 | Jul. 19, 2014 | Thailand |
| KU729217 | BeH823339 | Asian | 10645 | 2015 | 2015 | Brazil |
| KX197192 | ZIKV/H.sapiens/Brazil/PE243/2015 | Asian | 10807 | 2015 | 2015 | Brazil |
| KX280026 | Paraiba_01 | Asian | 10807 | 2015 | 2015 | Brazil |
| KU820897 | FLR | Asian | 10807 | 2015 | December 2015 | Colombia |
| KU758877 | 17271 | Asian | 10364 | 2015 | December 2015 | French Guiana |
| KX694534 | HND/R103451/2015 | Asian | 10772 | 2015 | Jan. 06, 2015 | Honduras |
| KU647676 | MRS_OPY_Martinique_PaRi_2015 | Asian | 10617 | 2015 | December 2015 | Martinique |
| KX247632 | MEX_I_7 | Asian | 10777 | 2015 | November 2015 | Mexico |
| KX156774 | PAN/CDC-259359_V1-V3/2015 | Asian | 10771 | 2015 | Dec. 18, 2015 | Panama |
| KX087101 | PRI/PRVABC59/2015 | Asian | 10778 | 2015 | December 2015 | Puerto Rico |
| KU312313 | Z1106032 | Asian | | 2015 | 2015 | Suriname |
| KX051562 | SV0010/15 | Asian | 10800 | 2015 | Jan. 16, 2015 | Thailand |
| KX806557 | TS17-2016 | Asian | 10807 | 2016 | February 2016 | Australia |
| KU926309 | Rio-U1 | Asian | 10807 | 2016 | Jan. 14, 2016 | Brazil |
| KY014301 | BRA/2016/FC-DQ107D1-URI | Asian | 10361 | 2016 | Apr. 13, 2016 | Brazil |
| KY014307 | BRA/2016/FC-DQ49D1-PLA | Asian | 10455 | 2016 | Mar. 28, 2016 | Brazil |
| KU740184 | GD01 | Asian | 10574 | 2016 | February 2016 | China |
| KU820899 | ZJ03 | Asian | 10805 | 2016 | Feb. 17, 2016 | China |
| KY927808 | ZZ-1 | Asian | 10633 | 2016 | September 2016 | China |
| KY967711 | SY01_2016 | Asian | 10632 | 2016 | Nov. 01, 2016 | China |
| KX247646 | COL/UF-1/2016 | Asian | 10808 | 2016 | Feb. 09, 2016 | Colombia |
| KX766028 | R114916 | Asian | 10680 | 2016 | Jun. 06, 2016 | Dominican Republic |
| KY014305 | DOM/2016/BB-0076-SER | Asian | 10366 | 2016 | Apr. 05, 2016 | Dominican Republic |
| KX879604 | EcEs089_16 | Asian | 10810 | 2016 | April 2016 | Ecuador |
| KX262887 | 103451 | Asian | 10806 | 2016 | Jan. 06, 2016 | Honduras |
| KU853012 | Dominican Republic/2016/PD1 | Asian | 10636 | 2016 | Feb. 01, 2016 | Italy |
| KY785419 | JAM/2016/WI-JM6-SER | Asian | 10599 | 2016 | Jun. 13, 2016 | Jamaica |
| LC190723 | ZIKV/Hu/Yokohama/1/2016 | Asian | 10786 | 2016 | May 20, 2016 | Japan |
| LC191864 | ZIKV/Hu/Chiba/S36/2016 | Asian | 10807 | 2016 | Apr. 21, 2016 | Japan |
| KY785451 | MTQ/2016/FL-001-SAL | Asian | 10345 | 2016 | Mar. 22, 2016 | Martinique |
| KU922923 | MEX/InDRE/Lm/2016 | Asian | 10617 | 2016 | Feb. 25, 2016 | Mexico |
| KX421194 | Nica2-16 | Asian | 10580 | 2016 | Jan. 13, 2016 | Nicaragua |
| KX198135 | PAN/BEI-259634_V4/2016 | Asian | 10778 | 2016 | 2016 | Panama |
| KY693678 | FPI15198/PERU/Loreto/2016 | Asian | 10666 | 2016 | Jun. 28, 2016 | Peru |
| KY785464 | PRI/2016/MA-WGS16-004-SER | Asian | 10453 | 2016 | Apr. 13, 2016 | Puerto Rico |
| KX813683 | ZKA-16-097 | Asian | 10484 | 2016 | Aug. 27, 2016 | Singapore |
| KX827309 | ZKA-16-291 | Asian | 10526 | 2016 | Aug. 28, 2016 | Singapore |
| KY348640 | SL1602 | Asian | 10807 | 2016 | Jan. 22, 2016 | Suriname |
| KY272987 | SI-BKK01 | Asian | 10807 | 2016 | Aug. 30, 2016 | Thailand |
| KU870645 | FB-GWUH-2016 | Asian | 10798 | 2016 | Feb. 02, 2016 | USA |
| KX702400 | VEN/UF-1/2016 | Asian | 10808 | 2016 | Mar. 25, 2016 | Venezuela |
| KX520666 | HS-2015-BA-01 | Asian | 10538 | August 2015 | August 2015 | Brazil |
| EU545988 | FSM | -N/A- | 10272 | 2007 | June 2007 | Micronesia |
| KU365777 | BeH818995 | -N/A- | 10662 | 2015 | 2015 | Brazil |
| KU501216 | 103344 | -N/A- | 10272 | 2015 | Dec. 01, 2015 | Guatemala |
| KU501215 | PRVABC59 | -N/A- | 10675 | 2015 | Dec. 01, 2015 | Puerto Rico |
| KU312312 | Z1106033 | -N/A- | 10374 | 2015 | Oct. 02, 2015 | Suriname |

1. A method of simultaneously detecting, differentiating, and/or quantifying Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, wherein the method comprising:
determining the presence of the target regions or fragments thereof selected from the group consisting of Non Structural protein 5 (NS5) of Zika virus, NS5 of DENV1, NS5 of DENV2, NS5 of DENV3, Capsid of DENV4, and E1 glycoprotein of CHIKV.

2. The method of claim 1, wherein the target regions or fragments thereof are encoded by the sequence SEQ ID NO: 1 (CHIKV E1 consensus sequence); SEQ ID NO: 2 (DENV1 NS5 48+22 seq consensus sequence); SEQ ID NO: 3 (DENV2 NS5 consensus sequence); SEQ ID NO: 4 (DENV3 NS5 consensus sequence); SEQ ID NO: 5 (DENV4 Capsid consensus sequence); and SEQ ID NO: 6 (ZIKV NS5 check_56seq consensus sequence).

3. The method of claim 1, wherein the detecting comprises performing reverse transcription polymerase chain reaction (RT-PCR).

4. The method of claim 1, wherein the primers and probe are selected from the group consisting of:
a ZIKV forward primer comprising a sequence at least 90% identical to CCTTGGATTCTTGAACGAGGATCAC (NS5_ZIKV-F/SEQ ID NO: 7);
a ZIKV reverse primer comprising a sequence at least 90% identical to GCTTCATTCTCCAGATCAAACCTGC (NS5_ZIKV-R/SEQ ID NO: 9) or GCTTCATTCTCTAGATCAAACCTGC (ZIKV-R1_T/SEQ ID NO: 32);
a ZIKV probe comprising a sequence at least 90% identical to TACCAGGAGGAAGGATGTATGCAG (5′-FAM NS5_ZIKV-P/ZEN/3′ IBFQ/SEQ ID NO: 8) or ACCAGGAGGAAAGATGTACGCAG (ZIKV_P1_AF/SEQ ID NO: 33);
a DENV1 first forward primer comprising a sequence at least 90% identical to GGCTGAAGAAAGTCACAGAAG (NS5_ D1-F_A/SEQID NO: 10);
a DENV1 second forward primer comprising a sequence at least 90% identical to GGCTGAAGAAAGTCACTGAAG (NS5_ D1-F_T/SEQID NO: 11);
a DENV1 reverse primer comprising a sequence at least 90% identical to GAGGACTCACCAATATCACACAA (NS5_D1-R/SEQ ID NO: 13);
a DENV1 probe comprising a sequence at least 90% identical to ACCTATGGATGGAACCTAGTAAAGCT (5′-HEX NS5_D1/ZEN/3′ IBFQ/SEQID NO: 12);
a DENV3 forward primer comprising a sequence at least 90% identical to GCTCAGCCTCCTCCATGATAAATG (NS5_D3-F/SEQ ID NO: 14);
a DENV3 reverse primer comprising a sequence at least 90% identical to GGGTGTCCTGGTGTCCACTTTCTC (NS5_D3-R/SEQ ID NO: 17);
a DENV3 first probe comprising a sequence at least 90% identical to CATGGTGACACAGATGGCAATGAC (5′-Texas Red NS5_D3-P_T/3′ IBRQ/SEQID NO: 15);
a DENV3 second probe comprising a sequence at least 90% identical to CACGGTGACACAGATGGCAATGAC (5′-Texas Red NS5_D3-P_C/3′ IBRQ/SEQID NO: 16);
a CHIKV forward primer comprising a sequence at least 90% identical to GGCGCCTACTGCTTCTGCGAC (E1_CHIKV-F1/SEQ ID NO: 18);
a CHIKV reverse primer comprising a sequence at least 90% identical to TTGGTAAAGGACGCGGAGCTTAGC (E1_CHIKV-R1/SEQ ID NO: 21);
a CHIKV first probe comprising a sequence at least 90% identical to AGCGAAGCACATGTGGAGAAGTCC (5′-FAM E1_CHIKV-P1_T/ZEN/3′ IBFQ/SEQ ID NO: 19);
a CHIKV second probe comprising a sequence at least 90% identical to AGCGAAGCACACGTGGAGAAGTCC (5′-FAM E1_CHIKV-P1_C/ZEN/3′ IBFQ/SEQ ID NO: 20);
a DENV2 forward primer comprising a sequence at least 90% identical to ACACAGATGGCAATGACAGACACG (NS5_D2-F2/SEQ ID NO: 22);
a DENV2 reverse primer comprising a sequence at least 90% identical to CCAAGGCTGCATTGCTTCTCAC (NS5_D2-R2/SEQ ID NO: 24);
a DENV2 probe comprising a sequence at least 90% identical to TGGAAAGAACTAGGAAAGAAAAAGACAC (5′-HEX NS5_D2-P2/ZEN/3′ IBFQ/SEQ ID NO: 23);
a DENV4 first forward primer comprising a sequence at least 90% identical to TGGTTAGACCACCTTTCAATATG (C_D4-F1.2_T/SEQID NO: 25);
a DENV4 second forward primer comprising a sequence at least 90% identical to TGGCTAGACCACCTTTCAATATG (C_D4-F1.2_C/SEQID NO: 26);
a DENV4 reverse primer comprising a sequence at least 90% identical to TGCTAGCACCATCCGTAA (C_D4-R1.2/SEQ ID NO: 28);
a DENV4 probe comprising a sequence at least 90% identical to CCTCAAGGGTTGGTGAAGAGATTC (5′-Texas Red C_D4-P1/3′ IBRQ/SEQ ID NO: 27); and
a combination thereof.

5. The method of claim 1, wherein the primers and probes are conjugated with a detectable label. In some examples, the detectable label may include, but is not limited to a fluorophore, a quencher, or a combination thereof

6. The method of claim 1, wherein the sample is selected from the group consisting of whole blood, serum, plasma, cerebrospinal fluid, urine, and amniotic fluid.

7. The method of claim 1, wherein the sample is whole blood.

8. The method of of claim 1, wherein the sample is whole blood treated with EDTA.

9-11. (canceled)

12. A method for detecting and/or differentiating and/or quantifying virus selected from the group consisting of Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, the method comprising:
subjecting the sample to a reverse transcription polymerase chain reaction (RT-PCR) using primers and a probe specific for CHIKV E1 glycoprotein, primers and a probe specific for DENV1 Non Structural protein 5 (NS5), primers and a probe specific for DENV2 NS5, primers and a probe specific for DENV3 NS5, primers and a probe specific for DENV4 capsid, and primers and a probe specific for ZIKV NS5.

13. The method of claim 12, wherein the primers and probes comprise:
a ZIKV forward primer comprising a sequence at least 90% identical to CCTTGGATTCTTGAACGAGGATCAC (SEQ ID NO: 7);
a ZIKV reverse primer comprising a sequence at least 90% identical to GCTTCATTCTCCAGATCAAACCTGC (SEQ ID NO: 9) or GCTTCATTCTCTAGATCAAACCTGC (SEQ ID NO: 32);
a ZIKV probe comprising a sequence at least 90% identical to TACCAGGAGGAAGGATGTATGCAG (SEQ ID NO: 8) or ACCAGGAGGAAAGATGTACGCAG (SEQ ID NO: 33);
a DENV1 first forward primer comprising a sequence at least 90% identical to GGCTGAAGAAAGTCACAGAAG (SEQ ID NO: 10);
a DENV1 second forward primer comprising a sequence at least 90% identical to GGCTGAAGAAAGTCACTGAAG (SEQ ID NO: 11);
a DENV1 reverse primer comprising a sequence at least 90% identical to GAGGACTCACCAATATCACACAA (SEQ ID NO: 13);
a DENV1 probe comprising a sequence at least 90% identical to ACCTATGGATGGAACCTAGTAAAGCT (SEQ ID NO: 12);
a DENV3 forward primer comprising a sequence at least 90% identical to GCTCAGCCTCCTCCATGATAAATG (SEQ ID NO: 14);
a DENV3 reverse primer comprising a sequence at least 90% identical to GGGTGTCCTGGTGTCCACTTTCTC (SEQ ID NO: 17);
a DENV3 first probe comprising a sequence at least 90% identical to CATGGTGACACAGATGGCAATGAC (SEQ ID NO: 15);
a DENV3 second probe comprising a sequence at least 90% identical to CACGGTGACACAGATGGCAATGAC (SEQ ID NO: 16);
a CHIKV forward primer comprising a sequence at least 90% identical to GGCGCCTACTGCTTCTGCGAC (SEQ ID NO: 18);
a CHIKV reverse primer comprising a sequence at least 90% identical to TTGGTAAAGGACGCGGAGCTTAGC (SEQ ID NO: 21);
a CHIKV first probe comprising a sequence at least 90% identical to AGCGAAGCACATGTGGAGAAGTCC (SEQ ID NO: 19);
a CHIKV second probe comprising a sequence at least 90% identical to AGCGAAGCACACGTGGAGAAGTCC (SEQ ID NO: 20);
a DENV2 forward primer comprising a sequence at least 90% identical to ACACAGATGGCAATGACAGACACG (SEQ ID NO: 22);
a DENV2 reverse primer comprising a sequence at least 90% identical to CCAAGGCTGCATTGCTTCTCAC (SEQ ID NO: 24);
a DENV2 probe comprising a sequence at least 90% identical to TGGAAAGAACTAGGAAAGAAAAAGACAC (SEQ ID NO: 23);
a DENV4 first forward primer comprising a sequence at least 90% identical to TGGTTAGACCACCTTTCAATATG (SEQ ID NO: 25);
a DENV4 second forward primer comprising a sequence at least 90% identical to TGGCTAGACCACCTTTCAATATG (SEQ ID NO: 26);
a DENV4 reverse primer comprising a sequence at least 90% identical to TGCTAGCACCATCCGTAA (SEQ ID NO: 28); and
a DENV4 probe comprising a sequence at least 90% identical to CCTCAAGGGTTGGTGAAGAGATTC (SEQ ID NO: 27).

14. The method of claim 12, wherein:
the ZIKV forward primer comprising
(SEQ ID NO: 7)
CCTTGGATTCTTGAACGAGGATCAC;
the ZIKV reverse primer comprising
(SEQ ID NO: 9)
GCTTCATTCTCCAGATCAAACCTGC
or
(SEQ ID NO: 32)
GCTTCATTCTCTAGATCAAACCTGC;
the ZIKV probe comprising
(SEQ ID NO: 8)
TACCAGGAGGAAGGATGTATGCAG
Or
(SEQ ID NO: 33)
ACCAGGAGGAAAGATGTACGCAG;
the DENV1 first forward primer comprising
(SEQ ID NO: 10)
GGCTGAAGAAAGTCACAGAAG;
the DENV1 second forward primer comprising
(SEQ ID NO: 11)
GGCTGAAGAAAGTCACTGAAG;
the DENV1 reverse primer comprising
(SEQ ID NO: 13)
GAGGACTCACCAATATCACACAA;
the DENVI probe comprising
(SEQ ID NO: 12)
ACCTATGGATGGAACCTAGTAAAGCT;
the DENV3 forward primer comprising
(SEQ ID NO: 14)
GCTCAGCCTCCTCCATGATAAATG;
the DENV3 reverse primer comprising
(SEQ ID NO: 17)
GGGTGTCCTGGTGTCCACTTTCTC;
the DENV3 first probe comprising
(SEQ ID NO: 15)
CATGGTGACACAGATGGCAATGAC;
the DENV3 second probe comprising
(SEQ ID NO: 16)
CACGGTGACACAGATGGCAATGAC;
the CHIKV forward primer comprising
(SEQ ID NO: 18)
GGCGCCTACTGCTTCTGCGAC;
the CHIKV reverse primer comprising
(SEQ ID NO: 21)
TTGGTAAAGGACGCGGAGCTTAGC;
the CHIKV first probe comprising
(SEQ ID NO: 19)
AGCGAAGCACATGTGGAGAAGTCC;
the CHIKV second probe comprising
(SEQ ID NO: 20)
AGCGAAGCACACGTGGAGAAGTCC;
the DENV2 forward primer comprising
(SEQ ID NO: 22)
ACACAGATGGCAATGACAGACACG;
the DENV2 reverse primer comprising
(SEQ ID NO: 24)
CCAAGGCTGCATTGCTTCTCAC;
the DENV2 probe comprising
(SEQ ID NO: 23)
TGGAAAGAACTAGGAAAGAAAAAGACAC;
the DENV4 first forward primer comprising
(SEQ ID NO: 25)
TGGTTAGACCACCTTTCAATATG;
the DENV4 second forward primer comprising
(SEQ ID NO: 26)
TGGCTAGACCACCTTTCAATATG;
the DENV4 reverse primer comprising
(SEQ ID NO: 28)
TGCTAGCACCATCCGTAA;
and
the DENV4 probe comprising
(SEQ ID NO: 27)
CCTCAAGGGTTGGTGAAGAGATTC.

15. The method of claim 1, wherein the primers and probes comprise a detectable label.

16. The method of claim 15, wherein the detectable label comprises a fluorophore, a quencher, or a combination thereof

17. A kit for detecting Chikungunya virus (CHIKV), Dengue virus serotype-1 (DENV1), Dengue virus serotype-2 (DENV2), Dengue virus serotype-3 (DENV3), Dengue virus serotype-4 (DENV4) and Zika virus (ZIKV) in a sample, comprising:
an agent specific for detecting CHIKV E1 glycoprotein, an agent specific for detecting DENV1 Non Structural protein 5 (NS5), an agent specific for detecting DENV2 NS5, an agent specific for detecting DENV3 NS5, an agent specific for detecting DENV4 capsid, and an agent specific for detecting ZIKV NS5.

18. The kit of claim 17, comprising
an agent for detecting a region or fragment thereof having 80% sequence identity to SEQ ID NO: 1; an agent for detecting a region or fragment thereof having 80% sequence identity to SEQ ID NO: 2; an agent for detecting a region or fragment thereof having 80% sequence identity to SEQ ID NO: 3; an agent for detecting a region or fragment thereof having 80% sequence identity to SEQ ID NO: 4; an agent for detecting a region or fragment thereof having 80% sequence identity to SEQ ID NO: 5; and an agent for detecting a region or fragment thereof having 80% sequence identity to SEQ ID NO: 6.

19. The kit of claim 17, wherein the agent comprises primers and probes comprising:
a ZIKV forward primer comprising a sequence at least 90% identical to CCTTGGATTCTTGAACGAGGATCAC (SEQ ID NO: 7);
a ZIKV reverse primer comprising a sequence at least 90% identical to GCTTCATTCTCCAGATCAAACCTGC (SEQ ID NO: 9) or GCTTCATTCTCTAGATCAAACCTGC (SEQ ID NO: 32);
a ZIKV probe comprising a sequence at least 90% identical to TACCAGGAGGAAGGATGTATGCAG (SEQ ID NO: 8) or ACCAGGAGGAAAGATGTACGCAG (SEQ ID NO: 33);
a DENV1 first forward primer comprising a sequence at least 90% identical to GGCTGAAGAAAGTCACAGAAG (SEQ ID NO: 10);
a DENV1 second forward primer comprising a sequence at least 90% identical to GGCTGAAGAAAGTCACTGAAG (SEQ ID NO: 11);
a DENV1 reverse primer comprising a sequence at least 90% identical to GAGGACTCACCAATATCACACAA (SEQ ID NO: 13);
a DENV1 probe comprising a sequence at least 90% identical to ACCTATGGATGGAACCTAGTAAAGCT (SEQ ID NO: 12);
a DENV3 forward primer comprising a sequence at least 90% identical to GCTCAGCCTCCTCCATGATAAATG (SEQ ID NO: 14);
a DENV3 reverse primer comprising a sequence at least 90% identical to GGGTGTCCTGGTGTCCACTTTCTC (SEQ ID NO: 17);
a DENV3 first probe comprising a sequence at least 90% identical to CATGGTGACACAGATGGCAATGAC (SEQ ID NO: 15);
a DENV3 second probe comprising a sequence at least 90% identical to CACGGTGACACAGATGGCAATGAC (SEQ ID NO: 16);
a CHIKV forward primer comprising a sequence at least 90% identical to GGCGCCTACTGCTTCTGCGAC (SEQ ID NO: 18);
a CHIKV reverse primer comprising a sequence at least 90% identical to TTGGTAAAGGACGCGGAGCTTAGC (SEQ ID NO: 21);
a CHIKV first probe comprising a sequence at least 90% identical to AGCGAAGCACATGTGGAGAAGTCC (SEQ ID NO: 19);
a CHIKV second probe comprising a sequence at least 90% identical to AGCGAAGCACACGTGGAGAAGTCC (SEQ ID NO: 20);
a DENV2 forward primer comprising a sequence at least 90% identical to ACACAGATGGCAATGACAGACACG (SEQ ID NO: 22);
a DENV2 reverse primer comprising a sequence at least 90% identical to CCAAGGCTGCATTGCTTCTCAC (SEQ ID NO: 24);
a DENV2 probe comprising a sequence at least 90% identical to TGGAAAGAACTAGGAAAGAAAAAGACAC (SEQ ID NO: 23);
a DENV4 first forward primer comprising a sequence at least 90% identical to TGGTTAGACCACCTTTCAATATG (SEQ ID NO: 25);
a DENV4 second forward primer comprising a sequence at least 90% identical to TGGCTAGACCACCTTTCAATATG (SEQ ID NO: 26);
a DENV4 reverse primer comprising a sequence at least 90% identical to TGCTAGCACCATCCGTAA (SEQ ID NO: 28); and
a DENV4 probe comprising a sequence at least 90% identical to CCTCAAGGGTTGGTGAAGAGATTC (SEQ ID NO: 27).
